(19) United States
(12) Patent Application Publication
(10) Pub. No.: US 2026/0055061 A1
(43) Pub. Date: Feb. 26, 2026

(54) HALOPHTHALIMIDE COMPOUNDS AND METHODS OF USE AGAINST TBI, INFLAMMATORY DISORDER, AUTOIMMUNE DISORDER, NEURODEGENERATIVE DISEASE OR VIRAL INFECTION

(71) Applicants: The United States of America, as represented by the Secretary, Department of Health & Human Services, Bethesda, MD (US); AevisBio, Inc., Gaithersburg, MD (US)

(72) Inventors: Nigel H. Greig, Phoenix, MD (US); Weiming Luo, Lutherville, MD (US); David Tweedie, Joppa, MD (US); Michael T. Scerba, Baltimore, MD (US); Daniela Lecca, S. Anna Arresi (IT); Shih Chang Hsueh, Taipei (TW); Dong Seok Kim, Daejeon (KR)

(73) Assignees: The United States of America, as represented by the Secretary, Department of Health & Human Services, Bethesda, MD (US); AevisBio, Inc., Gaithersburg, MD (US)

(21) Appl. No.: 19/102,830

(22) PCT Filed: Aug. 7, 2023
(86) PCT No.: PCT/US2023/029602

§ 371 Date: Feb. 10, 2025

Related U.S. Application Data
(60) Provisional application No. 63/397,235, filed on Aug. 11, 2022.

(51) Int. Cl. C07D 209/48; A61K 31/4035; A61K 31/454; A61P 25/00; A61P 31/14; C07D 401/04

(52) U.S. Cl. CPC C07D 209/48; A61K 31/4035; A61K 31/454; A61P 25/00; A61P 31/14; C07D 401/04

(57) ABSTRACT

Halophthalimides are disclosed. The halophthalimides may inhibit TNF-α activity, TNF-α synthesis, inflammation, inducible nitric oxide synthase, SARS-CoV-2 virus, or any combination thereof. The halophthalimides may be administered to a subject with a traumatic brain injury, an inflammatory disorder, an autoimmune disorder, a neurodegenerative disease, a viral infection, or any combination thereof. The disclosed halophthalimides have a structure according to Formula I, or a stereoisomer or pharmaceutically acceptable salt, solvate, or hydrate thereof,

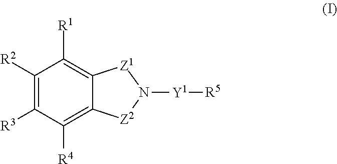

where R⁵ is

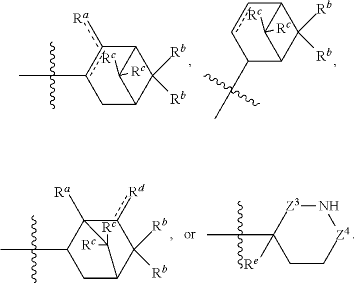

At least one of R²-R⁴ or R^{e} is halo.

### CROSS-REFERENCE TO RELATED APPLICATION

This application claims the benefit of the earlier filing date of U.S. Provisional Application No. 63/397,235, filed Aug. 11, 2022, which is incorporated by reference in its entirety herein.

### ACKNOWLEDGMENT OF GOVERNMENT SUPPORT

This invention was made with government support under Z01-AG-000994 awarded by the National Institutes of Health, National Institute on Aging. The government has certain rights in the invention.

### PARTIES TO JOINT RESEARCH AGREEMENT

This invention was made under a cooperative research and development agreement (CRADA) between the U.S. Government and AevisBio, Inc.

### FIELD

This disclosure concerns halophthalimides, pharmaceutical compositions including the halophthalimides, and methods of using the halophthalimides.

### SUMMARY

Halophthalimides are disclosed. Pharmaceutical compositions including the halophthalimides and methods of using the halophthalimides also are disclosed.

In some embodiments, a halophthalimide is a compound according to Formula I, or a stereoisomer or pharmaceutically acceptable salt, solvate, or hydrate thereof:

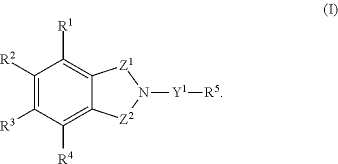

R¹ is —X, —N(R′)(R″), —NO₂, or —OH, wherein X is halo, and R′ and R″ independently are H or C₁-C₃ alkyl. R²-R⁴ independently are —X or —H. R⁵ is

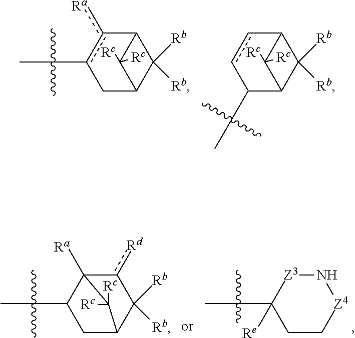

where each bond represented by “ [symbol] ” is a single or double bond as needed to satisfy valence requirements. R^{a} is —H, —CH₃, —CH₂OH, or ═CH₂. R^{b} and R^{c} independently are —H or —CH₃. R^{d} is —H, —OH, or ═O. R^{e} is —X, —H, or C₁-C₃ alkyl. Y¹ is a bond, —CH₂—, or —CH(CH₃)—. Z¹-Z⁴ independently are C(O) or C(S). At least one of R²-R⁴ or R^{e} is —X. In some embodiments, if R⁵ is

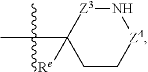

then (i) R¹ is —OH, or (ii) Y¹ is —CH₂— or —CH(CH₃)—, or (iii) R¹ is —N(R′)(R″) or —NO₂, at least one of R²-R⁴ is —X, R^{e} is —X or C₁-C₃ alkyl. In any of the foregoing or following embodiments, X may be F.

In some embodiments, R⁵ is a bridged carbocycle as shown above, and (i) Z¹ and Z² are C(O) and R¹-R⁴ are —F, or (ii) Z¹ and Z² are C(O), R¹ and R⁴ are —F, and R² and R³ are —H.

In some embodiments, R⁵ is

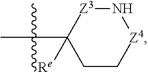

and at least one of Z¹-Z⁴ is C(S). In certain embodiments, Z¹ and Z⁴ are C(S). In some implementations, R³ is —H, R^{e} is —H or —CH₃, one of R² and R⁴ is —F, and the other of R² and R⁴ is —H. In other implementations, R^{e} is —F, and R²-R⁴ are —H. In some examples, R¹ is —N(R′)(R″) or —NO₂.

A pharmaceutical composition includes a halophthalimide as disclosed herein, or a stereoisomer, pharmaceutically acceptable salt, solvate, or a hydrate thereof, and a pharmaceutically acceptable carrier.

A method for inhibiting TNF-α activity, TNF-α synthesis, inflammation, inducible nitric oxide synthase (iNOS), SARS-CoV-2 virus, or any combination thereof, comprising contacting a cell with an effective amount of a halophthalimide as disclosed herein, or a pharmaceutically acceptable salt, solvate, or a hydrate thereof. In some embodiments, contacting the cell with an effective amount of the compound or pharmaceutically acceptable salt thereof comprises administering to a subject a therapeutically effective amount of the compound or pharmaceutically acceptable salt thereof or a therapeutically effective amount of a pharmaceutical composition comprising the compound or pharmaceutically acceptable salt thereof. In any of the foregoing embodiments, the subject may have a traumatic brain injury (TBI), an inflammatory disorder, an autoimmune disorder, a neurodegenerative disease, a viral infection, or any combination thereof.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an exemplary scheme for synthesizing monoterpenoid-substituted tetrafluorophthalimides.

FIG. 2 is an exemplary scheme for synthesizing monoterpenoid-substituted difluorophthalimides.

FIG. 3 shows X-ray crystallographic structures of several monoterpenoid-substituted tetra- and difluorophthalimides.

FIG. 4 is a bar graph comparing cytotoxicity (MTS), anti-nitrite, and anti-TNF-α activity from six pairs of substituted tetrafluorophthalimides and corresponding difluorophthalimides (drug treatment 1 μM; LPS=60 ng/ml).

FIG. 5 is a graph showing cell viability of the substituted tetrafluorophthalimides and corresponding difluorophthalimides of FIG. 3 (linear means linear forecast).

FIG. 6 is a bar graph showing anti-SARS-CoV-2 activity and cytotoxicity of several monoterpenoid-substituted tetra- and difluorophthalimides.

FIG. 7 is an exemplary scheme for synthesizing 7-fluorophthalimides.

FIG. 8 is an exemplary scheme for synthesizing 5-fluorophthalimides.

FIG. 9 is an X-ray crystallographic structure of a thionated aminofluorophthalimide.

FIGS. 10A-10C are bar graphs summarizing cell viability (FIG. 10A), TNF-α inhibition (FIG. 10B), and nitrite inhibition (FIG. 10C) by exemplary 7-fluorophthalimides.

FIGS. 11A-11C are bar graphs comparing cell-viability, TNF-α inhibition, and nitrite inhibition by nitro-substituted (FIG. 11A), isopropyl-substituted (FIG. 11B), and amino-substituted (FIG. 11C) 7-fluorophthalimides.

FIGS. 12A-12C are bar graphs summarizing cell viability (FIG. 12A), TNF-α inhibition (FIG. 12B), and nitrite inhibition (FIG. 12C) by exemplary pomalidomides, 7-fluorophthalimides and corresponding 5-fluorophthalimides.

FIG. 13 summarizes anti-nitrite activity across focused metabolic families of compounds.

FIG. 14 is a phase I microsomal analysis comparing a pomalidomide and its 7-fluoro analog.

FIGS. 15A-15F are bar graphs demonstrating that two exemplary compounds mitigate LPS-induced increases of nitrite and TNF-α in RAW 264.7 cell cultures. Tetrafluorobornylphthalimide (compound 14) was well tolerated and was without impact on cell viability (FIG. 15A). Compound 14 reduced nitrite expression at the lowest evaluated 100 nM concentration (FIG. 15B), whereas elevated levels of TNF-α were mitigated starting at 600 nM (FIG. 15C). Tetrafluoronorbornylphthalimide (compound 16) was likewise effective in decreasing levels of nitrite and TNF-α (FIGS. 15E, 15F, without affecting cell viability (FIG. 15D). *p<0.05; **p<0.01; ****p<0.0001 vs cnt-dmso group

FIGS. 16A-16F are bar graphs showing that compounds 14 (TFBP) and 16 (TFNBP) significantly decrease levels of TNF-α in plasma and cortex, as well as IFN-γ and IL-5 in plasma of LPS-challenged animals. FIGS. 16A and 16B show the effects on TNF-α in plasma and cortex, respectively. FIGS. 16C and 16D show the effects on IFN-γ in plasma and cortex, respectively. FIGS. 16E and 16F show the effects on IL-5 in plasma and cortex, respectively. *p<0.05, **p<0.01, ****p<0.0001 vs saline control group. #p<0.05, ##p<0.01. ###p<0.001, ####p<0.0001 vs LPS-treated group.

FIGS. 17A-17C are bar graphs showing that compound 14 (TFBP) partially improves motor function after TBI as evidenced by a beam walking test (FIG. 17A), immobility time in the beam walking test (FIG. 17B), and gait analysis (FIG. 17C). *p<0.05, ***p<0.001 vs control group; #p<0.05, ##p<0.01, ###p<0.001 vs CCI group).

FIGS. 18A-18C show that compound 14 (TFBP) significantly decreases cortical lesion volume in CCI-challenged mice. Effects on lesion size (FIG. 18A) and lateral ventricle size (FIG. 18B) are shown. FIG. 18C shows representative images of Giemsa-stained cortical sections. *p<0.05, ****p<0.0001 vs control; #p<0.05 vs CCI).

FIGS. 19A-19F show that compound 14 (TFBP) mitigates TBI-mediated expression of microglial cell activation. FIG. 19A shows representative images of Iba1+ cells at 40× magnification and their skeleton reconstructions through MotiQ software. Multiple parameters of Iba1+ cells morphology were analyzed, including ramification index (FIG. 19B), spanned area (FIG. 19C), number of branches (FIG. 19D), junctions (FIG. 19E) and endpoints (FIG. 19F). *p<0.05, ****p<0.0001 vs control group; #p<0.05, ##p<0.01, ###p<0.001, ####p<0.0001 vs CCI group; ++++p<0.0001 vs contralateral side.

FIGS. 20A-20C show interactions of pomalidomide, compound 14 (TFBP), and compound 16 (TFNBP) with cereblon; FIG. 20A shows a cereblon/BRD3 binding FRET assay; FIGS. 20B and 20C are a representative Western blot and graph, respectively showing effects on SALL4 expression levels.

FIGS. 21A-21C show that fluoro-3,6′-dithiopomalidomide (F-3,6′-DP, compound 30) significantly reduced LPS-induced proinflammatory cytokine TNF-α (FIG. 21A), chemokine (KC/GRO (CXCL1) (FIG. 21B), and IL-6 (FIG. 21C) in plasma, cortex, and hippocampus of mouse brain. **p<0.01, ***p<0.001 refers to the effects of LPS compared to the control value (CMC+Saline). #p<0.05, ##p<0.01 refers to the effect of drug treatments vs. CMC+LPS. Values are presented as mean±S.E.M., of n observations (CMC+Saline, n=4; LPS+CMC, n=4; LPS+F-3,6′-DP, n=5 in each dose).

FIG. 22 is a study timeline for treating TBI mice with compound 30 (F-3,6′-DP).

FIGS. 23A-23C show that compound 30 (F-3,6′-DP) reduced contusion volume after TBI. FIG. 23A shows representative Giemsa-stained coronal brain sections of the TBI-induced cavity in Sham (control without TBI), CMC+TBI, low dose (LD) (14.78 mg/kg) and high dose (HD) (29.57 mg/kg) treatments of F-3,6′-DP in TBI mice at 2 weeks post-TBI; FIG. 23B is a bar graph showing changes in lesion size; FIG. 23C is a bar graph showing changes in lateral ventricle size. *p<0.05, ****p<0.0001 refers to the effects of TBI compared to the control value (CMC+Sham). #p<0.05 refers to the effect of drug treatments vs. CMC+TBI. Values are presented as mean±S.E.M., of n observations (CMC+Sham, n=6; CMC+TBI, n=9; F-3,6′-DP (LD)+TBI, n=4; F-3,6′-DP (HD)+TBI, n=3).

FIGS. 24A-24C show that compound 30 (F-3,6′-DP) improved gait functional recovery as revealed by DiGi gait assessment; effects on the lift hind limb are shown. FIG. 24A is a bar graph showing changes in braking phase duration; FIG. 24B is a bar graph showing changes in propulsion phase; FIG. 24C is a bar graph showing variability of the paw angle. *p<0.05, **p<0.01, ***p<0.001 refers to the effects of TBI compared to the control value (CMC+Sham). #p<0.05, ##p<0.01 refers to the effect of drug treatments vs. CMC+TBI. Values are presented as mean±S.E.M., of n observations (CMC+Sham, n=5; CMC+TBI, n=5; F-3,6′-DP (LD)+TBI, n=5; F-3,6′-DP (HD)+TBI, n=4).

FIGS. 25A and 25B show that compound 30 (F-3,6′-DP) improved motor coordination and balance function on a beam walking test. FIG. 25A is a bar graph showing effects on the test time; FIG. 25B is a bar graph showing effects on contralateral foot falls during the test. **p<0.01 refers to the effects of TBI compared to the control value (CMC+Sham). #p<0.05 refers to the effect of drug treatments vs. CMC+TBI. Values are presented as mean±S.E.M., of n observations (CMC+Sham, n=5; CMC+TBI, n=5; F-3,6′-DP (LD)+TBI, n=5; F-3,6′-DP (HD)+TBI, n=4).

FIGS. 26A and 26B show that post-injury treatment with compound 30 (F-3,6′-DP) decreased GFAP-positive astrocytes at 2 weeks after TBI. FIG. 26A is immunofluorescence images of glial fibrillary acidic protein (GFAP) and Ionized calcium binding adaptor molecule 1 (Iba1) in cortical brain sections. GFAP, a marker for astrocytes, is showed in red. Iba1, a marker for microglia, is showed in green; FIG. 26B is bar graphs showing that treatment with F-3,6′-DP significantly reduced the amount of astrocytic elevated by TBI (B). *p<0.05, ****p<0.0001 refers to the effects of TBI compared to the control value (CMC+Sham). #p<0.05, ##p<0.01 refers to the effect of drug treatments vs. CMC+TBI. Values are presented as mean±S.E.M., of n observations (CMC+Sham, n=5; CMC+TBI, n=5; F-3,6′-DP (LD)+TBI, n=5; F-3,6′-DP (HD)+TBI, n=4).

FIGS. 27A-27F show the effects of compound 30 (F-3,6′-DP) at 2 weeks after TBI on microglial morphology in cortex. FIG. 27A shows maximum intensity projections of confocal z-stack Iba1 images (green) illustrating the microglial morphology in cortical sections and cell skeleton (red) of representative CMC+Sham, CMC+TBI, F-3,6′-DP (LD)+TBI and F-3,6′-DP (HD)+TBI microglial cells (highlighted); FIGS. 27B-27F are quantitative analyses of number of cell branches (FIG. 27B), cell process junctions (FIG. 27C), cell process end-points (FIG. 27D), ramification index (FIG. 27E), cell spanned area, (FIG. 27F) of microglia in each groups of animals.

FIGS. 28A-28G show interactions of pomalidomide and compound 30 (F-3,6′-DP) with cereblon; FIG. 28A shows a cereblon/BRD3 binding FRET assay; FIGS. 28B and 28C are a representative Western blot and graph, respectively showing degradation of SALL4; FIGS. 28D-28G show degradation of Ailos (FIGS. 28D, 28F) and Ikaros (FIGS. 28E, 28G). The relative expression level of each neo-substrate was quantified.*, p<0.05, **, p<0.01 ****, p<0.0001 vs. control (Con) value; #, p<0.05, ##, p<0.01, ####, p<0.0001 vs. pomalidomide value (Tukey's multiple comparisons test).

FIGS. 29A and 29B show that compound 30 significantly lowered LPS-induced elevations in nitrite and TNF-α levels. **, p<0.01, ***, p<0.001, ****, p<0.0001 vs. the control (LPS+Veh) group.

FIG. 30 shows the IMiD drug binding pocket (circle) in chain C of human cereblon and the top 3 pharmacores with their attributes.

FIG. 31 shows the docking of compound 30 with the pockets of FIG. 30

FIG. 32 shows the docking of TFNBP and TFBP (compounds 14 and 16) with the pocket of FIG. 30.

### DETAILED DESCRIPTION

Embodiments of halophthalimides are disclosed. Pharmaceutical compositions comprising one or more halophthalimides and methods of using the halophthalimides also are disclosed.

### I. Definitions

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). The presently disclosed compounds also include all isotopes of atoms present in the compounds, which can include, but are not limited to, deuterium, tritium, ¹⁸F, ¹⁴C, etc. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Alkyl: A hydrocarbon group having a saturated carbon chain. The chain may be cyclic, branched or unbranched and includes at least one sp³-hybridized carbon atom. Cyclic groups may be referred to as cycloalkyl. Examples, without limitation, of alkyl groups include methyl, ethyl, propyl, and isopropyl (2-propyl). Unless otherwise specified, an alkyl group may be substituted or unsubstituted.

Effective amount or therapeutically effective amount: An amount sufficient to provide a beneficial, or therapeutic, effect to a subject or a given percentage of subjects.

Excipient: A physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Inflammation: A protective response to harmful stimuli, often elicited by infection, irritation, injury or destruction of tissues. Inflammation can be provoked by physical, chemical, and/or biologic agents. Inflammation involves immune cells, blood vessels, and molecular mediators such as vasoactive amines, plasma endopeptidases, prostaglandins, neutrophil products, lymphocyte factors, and others. Some hormones are anti-inflammatory while others are proinflammatory. Signs of inflammation include pain, heat, redness, swelling, and/or loss of function.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more thalidomide analogs as disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutically acceptable salt: A biologically compatible salt of a disclosed compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19, which is incorporated herein by reference.) For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

Pharmaceutical composition: A composition that includes an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

Solvate: A complex formed by combination of solvent molecules with molecules or ions of a solute. The solvent can be an organic solvent, an inorganic solvent, or a mixture of both. Exemplary solvents include, but are not limited to, alcohols, such as methanol, ethanol, propanol; amides such as N,N-dialiphatic amides, such as N,N-dimethylformamide; tetrahydrofuran; alkylsulfoxides, such as dimethylsulfoxide; water; and combinations thereof. The compounds described herein can exist in un-solvated as well as solvated forms when combined with solvents, pharmaceutically acceptable or not, such as water, ethanol, and the like. Solvated forms of the presently disclosed compounds are within the scope of the embodiments disclosed herein.

Stereoisomers: Isomers that differ in the arrangement of their atoms in space are termed “stereoisomers”. Stereoisomers that are not mirror images of one another are termed “diastereomers” and those that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture.” When spatial orientation is not indicated in a chemical formula, the formula includes all possible spatial orientations.

Subject: An animal (human or non-human) subjected to a treatment, observation or experiment. Includes both human and veterinary subjects, including human and non-human mammals, such as rats, mice, cats, dogs, pigs, horses, cows, and non-human primates.

Treat(ing) or treatment: As used herein, these terms refer to ameliorating a disease or condition of interest in a patient or subject, particularly a human having the disease or condition of interest, and includes by way of example, and without limitation:
(i) inhibiting the disease or condition, for example, arresting or slowing its development;
(ii) relieving the disease or condition, for example, causing regression of the disease or condition or a symptom thereof; or
(iii) stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### II. Halophthalimides

Embodiments of the disclosed halophthalimides are compounds according to Formula I, or stereoisomers or pharmaceutically acceptable salts, solvates, or hydrates thereof:

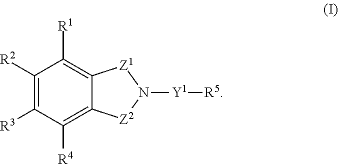

With respect to Formula I, R¹ is —X, —N(R′)(R″), —NO₂, or —OH, wherein X is halo, and R′ and R″ independently are H or C₁-C₃ alkyl. R²-R⁴ independently are —X or —H. R⁵ is

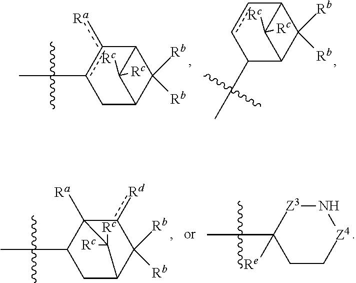

where each bond represented by “ [symbol] ” is a single or double bond as needed to satisfy valence requirements. R^{a} is —H, —CH₃, —CH₂OH, or ═CH₂. R^{b} and R^{c} independently are —H or —CH₃. R^{d} is —H, —OH, or ═O. R^{e} is —X, —H, or C₁-C₃ alkyl. Y¹ is a bond, —CH₂—, or —CH(CH₃)—. Z¹-Z⁴ independently are C(O) or C(S). At least one of R²-R⁴ or R^{e} is —X. In certain implementations, X is fluoro.

In some embodiments, if R⁵ is

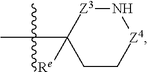

then (i) R¹ is —OH, or (ii) Y¹ is —CH₂— or —CH(CH₃)—, or (iii) R¹ is —NO₂, at least one of R²-R⁴ is —X, and at least one of Z¹-Z⁴ is C(S); or (iv) R¹ is —NH₂ and R^{e} is —X or C₁-C₃ alkyl; or (v) R¹ is —N(R′)(R″) or —NO₂, at least one of Z¹-Z⁴ is C(S), and R^{e} is —X or C₁-C₃ alkyl. In some implementations, if R⁵ is

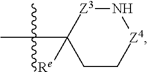

then at least one of R² and R⁴ is F, and (i) R¹ is —OH, or (ii) Y¹ is —CH₂— or —CH(CH₃)—, or (iii) R¹ is —N(R′)(R″) or —NO₂, at least one of R²-R⁴ is —X, and R^{e} is —X or C₁-C₃ alkyl. In some implementations, if R⁵ is

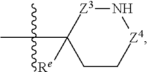

then at least one of Z³ and Z⁴ is C(S), and (i) R¹ is —OH, or (ii) Y¹ is —CH₂— or —CH(CH₃)—, or (iii) R¹ is —N(R′)(R″) or —NO₂, at least one of R²-R⁴ is —X, and R^{e} is —X or C₁-C₃ alkyl. In certain implementations, if R⁵ is

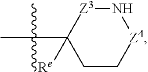

then at least one of R² and R⁴ is —F, at least one of Z³ and Z⁴ is C(S), and (i) R¹ is —OH, or (ii) Y¹ is —CH₂— or —CH(CH₃)—, or (iii) R¹ is —N(R′)(R″) or —NO₂, at least one of R²-R⁴ is —X, and R^{e} is —X or C₁-C₃ alkyl.

The compound according to formula I is not

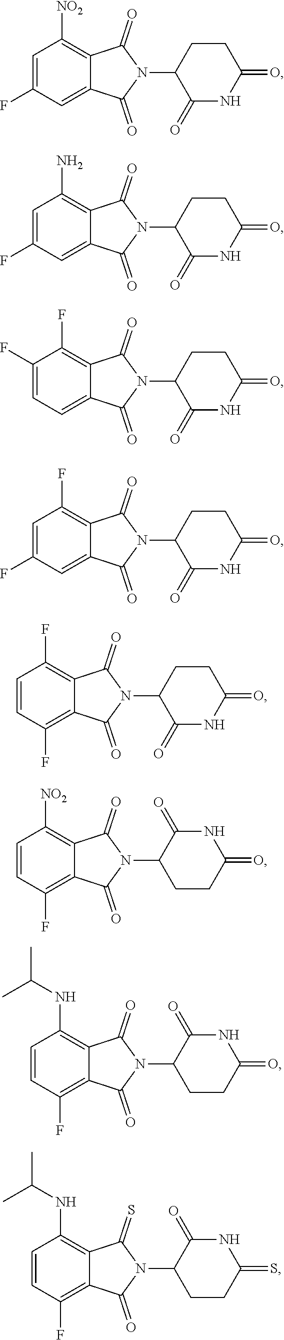

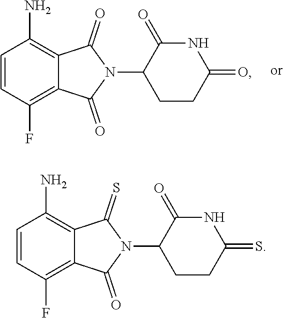

In formula I, R¹ is —X, —N(R′)(R″), —NO₂, or —OH, where X is halo, and R′ and R″ independently are H or C₁-C₃ alkyl. In some embodiments, R¹ is —F, —NH₂, —NH(CH₃), —NO₂, or —OH. In certain implementations, R¹ is —F, —NH₂, or —NH(CH₃).

R²-R⁴ independently are —X or —H. In some embodiments, X is fluoro. In one implementation, one of R²-R⁴ is —X, and the others of R²-R⁴ are —H. In certain examples, one of R² and R⁴ is —X, and the others of R²-R⁴ are —H. For example, R² may be —X, and R³ and R⁴ are —H; or R⁴ may be —X, and R² and R³ are —H. In an independent implementation, R¹-R⁴ are —X. In another independent implementation, R¹ and one of R²-R⁴ are —X, and the others of R²-R⁴ are —H. In some examples, R¹ and R⁴ are —X, and R² and R³ are —H.

R⁵ is

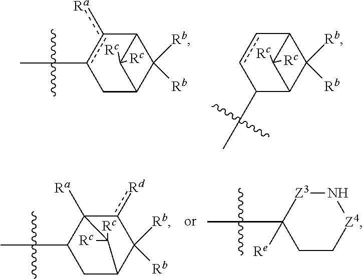

where each bond represented by “ [symbol] ” is a single or double bond as needed to satisfy valence requirements; R^{a} is —H, —CH₃, —CH₂OH, or ═CH₂; R^{b} and R^{c} independently are —H or —CH₃; R^{d} is —H, —OH, or ═O; and R^{e} is —X, —H, or C₁-C₃ alkyl. In some embodiments, R⁵ is

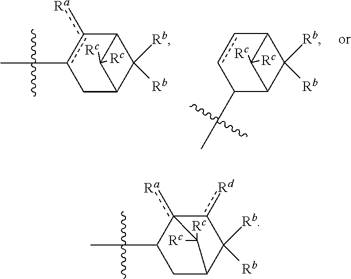

In some implementations, R^{a} is —H or —CH₃. In one embodiment, R^{a} is —CH₃, each R^{b} is —CH₃, and each R^{e} is —H. In an independent embodiment, R^{a} is —H, each R^{b} is —CH₃, and each R^{e} is —H. In another independent embodiment, R^{a}-R^{c} are all —H. In still another embodiment, R⁵ is

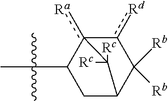

where R^{a} is —CH₃, each R^{b} is —H, each R^{c} is —CH₃, and R^{d} is H. In yet another independent embodiment, R⁵ is

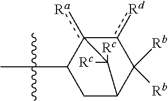

where R^{a} is —H, each R^{b} is —H, each R^{c} is —CH₃, and R^{d} is H. In another independent embodiment, R⁵ is

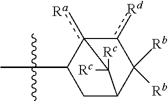

where R^{a}-R^{d} are all —H. In some examples, when R^{d-} is —OH or ═O, then R^{a} is —H. Representative examples of R⁵ bridged carbocycles include

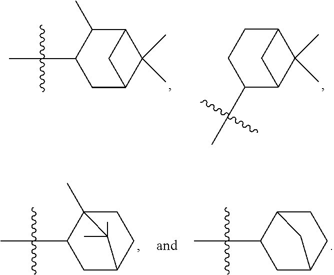

R⁵ includes stereocenters. In some implementations, R⁵ is

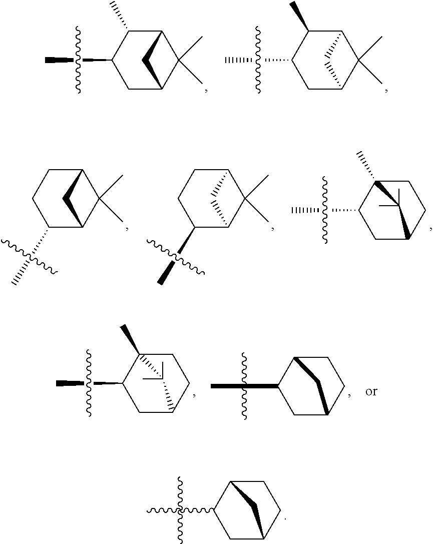

In some embodiments, R⁵ is

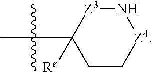

In certain implementations, R^{e} is —H, —CH₃, or —F. When R^{e} is other than —X, then at least one of R²-R⁴ is —X. If R^{e} is —X, then R²-R⁴ may be —H.

In any of the foregoing or following embodiments, Y¹ may be a bond, —CH₂—, or —CH(CH₃)—. In some embodiments, Y¹ is a bond. In some examples, R⁵ is a bridged carbocycle as described above, and Y¹ is —CH₂— or —CH(CH₃)—.

In any of the foregoing or following embodiments, Z¹-Z⁴ independently may be C(O) or C(S). In some embodiments, at least one of Z¹-Z⁴ is C(S). In one implementation, R⁵ is a bridged carbocycle, and Z¹ and Z² are C(O). In another implementation, R⁵ is a bridged carbocycle, and one of Z¹ and Z² is C(S), and the other of Z¹ and Z² is C(O). In one implementation, R⁵ is

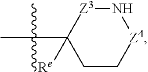

one or more of Z¹-Z⁴ is C(S) and the others of Z¹-Z⁴ are C(O). In an independent implementation, R⁵ is

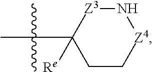

one of Z¹ and Z² is C(S) and one of Z³ and Z⁴ is C(S). In another independent implementation, R⁵ is

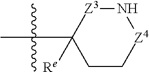

where R^{e} is —X or C₁-C₃ alkyl, and Z¹-Z⁴ are C(O). In yet another independent implementation, R⁵ is

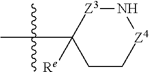

where R^{e} is —X or C₁-C₃ alkyl, one of Z¹-Z⁴ is C(S), and the others of Z¹-Z⁴ are C(O). In still another independent implementation, R⁵ is

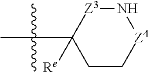

where R^{e} is —X or C₁-C₃ alkyl, one of Z¹ and Z² is C(S) and one of Z³ and Z⁴ is C(S).

In any of the foregoing or following embodiments, R⁵ may be

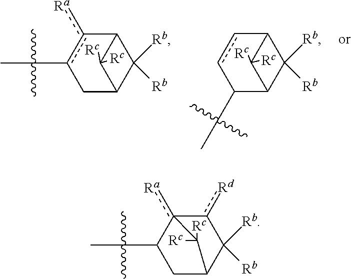

In some embodiments, (A) (i) one of R¹-R⁴ is —F, and the others of R¹-R⁴ are —H, or (ii) two of R¹-R⁴ are —F (R¹ and R², R¹ and R³, R¹ and R⁴, R² and R³, R² and R⁴, R³ and R⁴), and the others of R¹-R⁴ are —H, or (iii) one of R¹-R⁴ is —H and the others of R¹-R⁴ are —F, or (iv) R¹-R⁴ are —F; (B) (i) Z¹ and Z² are C(O) or (ii) one of Z¹ and Z² is C(O) and the other of Z¹ and Z² is C(S); and (C) Y¹ is a bond, —CH₂—, or —CH(CH₃)—. In certain embodiments, (i) Z¹ and Z² are C(O) and R¹-R⁴ are —F; or (ii) Z¹ and Z² are C(O), R¹ and R⁴ are —F, and R² and R³ are —H. In some implementations, R⁵ is

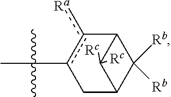

and (i) R^{a} and R^{b} are —CH₃ and R^{c} is —H; or (ii) R^{a} and R^{c} are H, and R^{b} is —CH₃; or (iii) R^{a} and R^{c} are —CH₃ and R^{b} is —H; or (iv) R^{a} is CH₂OH, R^{b} is —CH₃, and R^{c} is —H; or (v) R^{a} is ═CH₂, R^{b} is —CH₃, and R^{c} is —H. In some implementations, R⁵ is

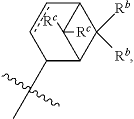

and (i) R^{b} is CH₃ and R^{c} is —H; or (ii) R^{b} is —H and R^{c} is —CH₃. In some implementations, R⁵ is

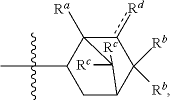

and (i) R^{a} and R^{b} are —CH₃, and R^{c} and R^{d} are —H; or (ii) R^{b} is —CH₃ and R^{a}, R^{c}, and R^{d} are —H; or (iii) R^{c} is —CH₃ and R^{a}, R^{b}, and R^{d} are —H; or (iv) R^{a} and R^{c} are —CH₃, and R^{b} and R^{d} are —H; or (v) R^{a} is —CH₂OH, R^{c} is —CH₃, and R^{b} and R^{d} are H; or (vi) R^{a} and R^{b} are —CH₃, R^{c} is —H, and R^{d} is —OH; or (vii) R^{b} is —CH₃, R^{a} and R^{c} are —H, and R^{d} is —OH; or (viii) R^{c} is —CH₃, R^{a} and R^{b} are —H, and R^{d} is —OH; or (ix) R^{a} and R^{c} are —CH₃, R^{b} is —H, and R^{d} is —OH; or R^{a} is —CH₂OH, R^{b} is —H, R^{c} is —CH₃, and R^{d} is —OH; or (x) R^{a} and R^{b} are —CH₃, R^{c} is —H, and R^{d} is ═O; or (xi) R^{a} and R^{c} are H, R^{b} is —CH₃, and R^{d} is ═O; or (xii) R^{a} and R^{b} are H, R^{c} is —CH₃, and R^{d} is ═O; or (xiii) R^{a} and R^{c} are —CH₃, R^{b} is —H, and R^{d} is ═O; or R^{a} is —CH₂OH, R^{b} is —H, R^{c} is —CH₃, and R^{d} is ═O.

In any of the foregoing or following embodiments, R⁵ may be

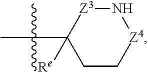

and at least one of Z¹-Z⁴ is C(S). In one such implementation, R³ is —H, R^{e} is —H or —CH₃, one of R² and R⁴ is —F, and the other of R² and R⁴ is —H. In another such implementation, R^{e} is —F, and R²-R⁴ are —H. In some implementations, R¹ is —NH₂, —NH (iPr), —NO₂, or —OH. In some embodiments, two of Z¹-Z⁴ are C(S). In certain implementations, one of Z¹ and Z² is C(S), and one of Z³ and Z⁴ is C(S). For example, Z¹ and Z³, Z¹ and Z⁴, Z² and Z³, or Z² and Z⁴ may be C(S), and the others of Z¹-Z⁴ are C(O).

In some embodiments, R⁵ is

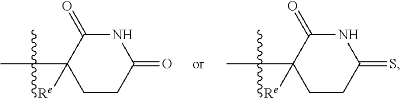

where R^{e} is —H, —F, or —CH₃. In some implementations, R¹ is —N(R′)(R″) or —NO₂; one of R² and R⁴ is —F, and the other of R² and R⁴ is —H; and R³ is H. In some examples, if R^{e} is —F, then R²-R⁴ are —H. In any of the foregoing arrangements, one of Z¹ and Z² may be C(S).

Exemplary, non-limiting examples of compounds according to Formula I are described in Tables 1-4.

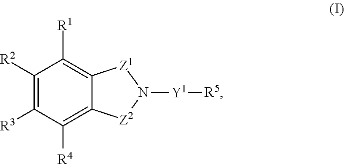

where Y¹ is a bond, —CH₂—, or —CH(CH₃)—

TABLE 1
R5 is

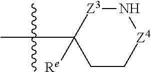

| | R¹ | R² | R³ | R⁴ | Z¹ | Z² | Z³ | Z⁴ | R^{e} |
|---|---|---|---|---|---|---|---|---|---|
| 1-1 | NH₂ | H | H | F | O | O | O | S | H |
| 1-2 | NH₂ | F | H | H | O | O | O | S | H |
| 1-3 | NH₂ | H | H | F | S | O | O | S | H |
| 1-4 | NH₂ | F | H | H | S | O | O | S | H |
| 1-5 | NH₂ | H | H | F | O | S | O | S | H |
| 1-6 | NH₂ | F | H | H | O | S | O | S | H |
| 1-7 | NH₂ | H | H | F | O | O | S | O | H |
| 1-8 | NH₂ | F | H | H | O | O | S | O | H |
| 1-9 | NH₂ | H | H | F | S | O | S | O | H |
| 1-10 | NH₂ | F | H | H | S | O | S | O | H |
| 1-11 | NH₂ | H | H | F | O | S | S | O | H |
| 1-12 | NH₂ | F | H | H | O | S | S | O | H |
| 1-13 | NH₂ | H | H | F | O | O | O | S | CH₃ |
| 1-14 | NH₂ | F | H | H | O | O | O | S | CH₃ |
| 1-15 | NH₂ | H | H | F | S | O | O | S | CH₃ |
| 1-16 | NH₂ | F | H | H | S | O | O | S | CH₃ |
| 1-17 | NH₂ | H | H | F | O | S | O | S | CH₃ |
| 1-18 | NH₂ | F | H | H | O | S | O | S | CH₃ |
| 1-19 | NH₂ | H | H | F | O | O | S | O | CH₃ |
| 1-20 | NH₂ | F | H | H | O | O | S | O | CH₃ |
| 1-21 | NH₂ | H | H | F | S | O | S | O | CH₃ |
| 1-22 | NH₂ | F | H | H | S | O | S | O | CH₃ |
| 1-23 | NH₂ | H | H | F | O | S | S | O | CH₃ |
| 1-24 | NH₂ | F | H | H | O | S | S | O | CH₃ |
| 1-25 | NH₂ | H | H | H | O | O | O | O | F |
| 1-26 | NH₂ | H | H | H | O | O | S | O | F |
| 1-27 | NH₂ | H | H | H | O | S | S | O | F |
| 1-28 | NH₂ | H | H | H | S | O | S | O | F |
| 1-29 | NH(iPr) | H | H | F | O | O | O | S | H |
| 1-30 | NH(iPr) | F | H | H | O | O | O | S | H |
| 1-31 | NH(iPr) | H | H | F | S | O | O | S | H |
| 1-32 | NH(iPr) | F | H | H | S | O | O | S | H |
| 1-33 | NH(iPr) | H | H | F | O | S | O | S | H |
| 1-34 | NH(iPr) | F | H | H | O | S | O | S | H |
| 1-35 | NH(iPr) | H | H | F | O | O | S | O | H |
| 1-36 | NH(iPr) | F | H | H | O | O | S | O | H |
| 1-37 | NH(iPr) | H | H | F | S | O | S | O | H |
| 1-38 | NH(iPr) | F | H | H | S | O | S | O | H |
| 1-39 | NH(iPr) | H | H | F | O | S | S | O | H |
| 1-40 | NH(iPr) | F | H | H | O | S | S | O | H |
| 1-41 | NH(iPr) | H | H | F | O | O | O | S | CH₃ |
| 1-42 | NH(iPr) | F | H | H | O | O | O | S | CH₃ |
| 1-43 | NH(iPr) | H | H | F | S | O | O | S | CH₃ |
| 1-44 | NH(iPr) | F | H | H | S | O | O | S | CH₃ |
| 1-45 | NH(iPr) | H | H | F | O | S | O | S | CH₃ |
| 1-46 | NH(iPr) | F | H | H | O | S | O | S | CH₃ |
| 1-47 | NH(iPr) | H | H | F | O | O | S | O | CH₃ |
| 1-48 | NH(iPr) | F | H | H | O | O | S | O | CH₃ |
| 1-49 | NH(iPr) | H | H | F | S | O | S | O | CH₃ |
| 1-50 | NH(iPr) | F | H | H | S | O | S | O | CH₃ |
| 1-51 | NH(iPr) | H | H | F | O | S | S | O | CH₃ |
| 1-52 | NH(iPr) | F | H | H | O | S | S | O | CH₃ |
| 1-53 | NH(iPr) | H | H | H | O | O | O | O | F |
| 1-54 | NH(iPr) | H | H | H | O | O | S | O | F |
| 1-55 | NH(iPr) | H | H | H | O | S | S | O | F |
| 1-56 | NH(iPr) | H | H | H | S | O | S | O | F |
| 1-57 | NO₂ | H | H | F | O | O | O | S | H |
| 1-58 | NO₂ | F | H | H | O | O | O | S | H |
| 1-59 | NO₂ | H | H | F | S | O | O | S | H |
| 1-60 | NO₂ | F | H | H | S | O | O | S | H |
| 1-61 | NO₂ | H | H | F | O | S | O | S | H |
| 1-62 | NO₂ | F | H | H | O | S | O | S | H |
| 1-63 | NO₂ | H | H | F | O | O | S | O | H |
| 1-64 | NO₂ | F | H | H | O | O | S | O | H |
| 1-65 | NO₂ | H | H | F | S | O | S | O | H |
| 1-66 | NO₂ | F | H | H | S | O | S | O | H |
| 1-67 | NO₂ | H | H | F | O | S | S | O | H |
| 1-68 | NO₂ | F | H | H | O | S | S | O | H |
| 1-69 | NO₂ | H | H | F | O | O | O | S | CH₃ |
| 1-70 | NO₂ | F | H | H | O | O | O | S | CH₃ |
| 1-71 | NO₂ | H | H | F | S | O | O | S | CH₃ |
| 1-72 | NO₂ | F | H | H | S | O | O | S | CH₃ |
| 1-73 | NO₂ | H | H | F | O | S | O | S | CH₃ |
| 1-74 | NO₂ | F | H | H | O | S | O | S | CH₃ |
| 1-75 | NO₂ | H | H | F | O | O | S | O | CH₃ |
| 1-76 | NO₂ | F | H | H | O | O | S | O | CH₃ |
| 1-77 | NO₂ | H | H | F | S | O | S | O | CH₃ |
| 1-78 | NO₂ | F | H | H | S | O | S | O | CH₃ |
| 1-79 | NO₂ | H | H | F | O | S | S | O | CH₃ |
| 1-80 | NO₂ | F | H | H | O | S | S | O | CH₃ |
| 1-81 | NO₂ | H | H | H | O | O | O | O | F |
| 1-82 | NO₂ | H | H | H | O | O | S | O | F |
| 1-83 | NO₂ | H | H | H | O | S | S | O | F |
| 1-84 | NO₂ | H | H | H | S | O | S | O | F |
| 1-85 | OH | H | H | F | O | O | O | S | H |
| 1-86 | OH | F | H | H | O | O | O | S | H |
| 1-87 | OH | H | H | F | S | O | O | S | H |
| 1-88 | OH | F | H | H | S | O | O | S | H |
| 1-89 | OH | H | H | F | O | S | O | S | H |
| 1-90 | OH | F | H | H | O | S | O | S | H |
| 1-91 | OH | H | H | F | O | O | S | O | H |
| 1-92 | OH | F | H | H | O | O | S | O | H |
| 1-93 | OH | H | H | F | S | O | S | O | H |
| 1-94 | OH | F | H | H | S | O | S | O | H |
| 1-95 | OH | H | H | F | O | S | S | O | H |
| 1-96 | OH | F | H | H | O | S | S | O | H |
| 1-97 | OH | H | H | F | O | O | O | S | CH₃ |
| 1-98 | OH | F | H | H | O | O | O | S | CH₃ |
| 1-99 | OH | H | H | F | S | O | O | S | CH₃ |
| 1-100 | OH | F | H | H | S | O | O | S | CH₃ |
| 1-101 | OH | H | H | F | O | S | O | S | CH₃ |
| 1-102 | OH | F | H | H | O | S | O | S | CH₃ |
| 1-103 | OH | H | H | F | O | O | S | O | CH₃ |
| 1-104 | OH | F | H | H | O | O | S | O | CH₃ |
| 1-105 | OH | H | H | F | S | O | S | O | CH₃ |
| 1-106 | OH | F | H | H | S | O | S | O | CH₃ |
| 1-107 | OH | H | H | F | O | S | S | O | CH₃ |
| 1-108 | OH | F | H | H | O | S | S | O | CH₃ |
| 1-109 | OH | H | H | H | O | O | O | O | F |
| 1-110 | OH | H | H | H | O | O | S | O | F |
| 1-111 | OH | H | H | H | O | S | S | O | F |
| 1-112 | OH | H | H | H | S | O | S | O | F |
| 1-113 | NH₂ | F | H | F | O | O | O | S | H |
| 1-114 | NH₂ | H | F | H | O | O | O | S | H |
| 1-115 | NH₂ | F | H | F | S | O | O | S | H |
| 1-116 | NH₂ | H | F | H | S | O | O | S | H |
| 1-117 | NH₂ | F | H | F | O | S | O | S | H |
| 1-118 | NH₂ | H | F | H | O | S | O | S | H |
| 1-119 | NH₂ | F | H | F | O | O | S | O | H |
| 1-120 | NH₂ | H | F | H | O | O | S | O | H |
| 1-121 | NH₂ | F | H | F | S | O | S | O | H |
| 1-122 | NH₂ | H | F | H | S | O | S | O | H |
| 1-123 | NH₂ | F | H | F | O | S | S | O | H |
| 1-124 | NH₂ | H | F | H | O | S | S | O | H |
| 1-125 | NH₂ | F | H | F | O | O | O | S | CH₃ |
| 1-126 | NH₂ | H | F | H | O | O | O | S | CH₃ |
| 1-127 | NH₂ | F | H | F | S | O | O | S | CH₃ |
| 1-128 | NH₂ | H | F | H | S | O | O | S | CH₃ |
| 1-129 | NH₂ | F | H | F | O | S | O | S | CH₃ |
| 1-130 | NH₂ | H | F | H | O | S | O | S | CH₃ |
| 1-131 | NH₂ | F | H | F | O | O | S | O | CH₃ |
| 1-132 | NH₂ | H | F | H | O | O | S | O | CH₃ |
| 1-133 | NH₂ | F | H | F | S | O | S | O | CH₃ |
| 1-134 | NH₂ | H | F | H | S | O | S | O | CH₃ |
| 1-135 | NH₂ | F | H | F | O | S | S | O | CH₃ |
| 1-136 | NH₂ | H | F | H | O | S | S | O | CH₃ |
| 1-137 | NH(iPr) | F | H | F | O | O | O | S | H |
| 1-138 | NH(iPr) | H | F | H | O | O | O | S | H |
| 1-139 | NH(iPr) | F | H | F | S | O | O | S | H |
| 1-140 | NH(iPr) | H | F | H | S | O | O | S | H |
| 1-141 | NH(iPr) | F | H | F | O | S | O | S | H |
| 1-142 | NH(iPr) | H | F | H | O | S | O | S | H |
| 1-143 | NH(iPr) | F | H | F | O | O | S | O | H |
| 1-144 | NH(iPr) | H | F | H | O | O | S | O | H |
| 1-145 | NH(iPr) | F | H | F | S | O | S | O | H |
| 1-146 | NH(iPr) | H | F | H | S | O | S | O | H |
| 1-147 | NH(iPr) | F | H | F | O | S | S | O | H |
| 1-148 | NH(iPr) | H | F | H | O | S | S | O | H |
| 1-149 | NH(iPr) | F | H | F | O | O | O | S | CH₃ |
| 1-150 | NH(iPr) | H | F | H | O | O | O | S | CH₃ |
| 1-151 | NH(iPr) | F | H | F | S | O | O | S | CH₃ |
| 1-152 | NH(iPr) | H | F | H | S | O | O | S | CH₃ |
| 1-153 | NH(iPr) | F | H | F | O | S | O | S | CH₃ |
| 1-154 | NH(iPr) | H | F | H | O | S | O | S | CH₃ |
| 1-155 | NH(iPr) | F | H | F | O | O | S | O | CH₃ |
| 1-156 | NH(iPr) | H | F | H | O | O | S | O | CH₃ |
| 1-157 | NH(iPr) | F | H | F | S | O | S | O | CH₃ |
| 1-157 | NH(iPr) | H | F | H | S | O | S | O | CH₃ |
| 1-159 | NH(iPr) | F | H | F | O | S | S | O | CH₃ |
| 1-160 | NH(iPr) | H | F | H | O | S | S | O | CH₃ |
| 1-161 | NO₂ | F | H | F | O | O | O | S | H |
| 1-162 | NO₂ | H | F | H | O | O | O | S | H |
| 1-163 | NO₂ | F | H | F | S | O | O | S | H |
| 1-164 | NO₂ | H | F | H | S | O | O | S | H |
| 1-165 | NO₂ | F | H | F | O | S | O | S | H |
| 1-166 | NO₂ | H | F | H | O | S | O | S | H |
| 1-167 | NO₂ | F | H | F | O | O | S | O | H |
| 1-168 | NO₂ | H | F | H | O | O | S | O | H |
| 1-169 | NO₂ | F | H | F | S | O | S | O | H |
| 1-170 | NO₂ | H | F | H | S | O | S | O | H |
| 1-171 | NO₂ | F | H | F | O | S | S | O | H |
| 1-172 | NO₂ | H | F | H | O | S | S | O | H |
| 1-173 | NO₂ | F | H | F | O | O | O | S | CH₃ |
| 1-174 | NO₂ | H | F | H | O | O | O | S | CH₃ |
| 1-175 | NO₂ | F | H | F | S | O | O | S | CH₃ |
| 1-176 | NO₂ | H | F | H | S | O | O | S | CH₃ |
| 1-177 | NO₂ | F | H | F | O | S | O | S | CH₃ |
| 1-178 | NO₂ | H | F | H | O | S | O | S | CH₃ |
| 1-179 | NO₂ | F | H | F | O | O | S | O | CH₃ |
| 1-180 | NO₂ | H | F | H | O | O | S | O | CH₃ |
| 1-181 | NO₂ | F | H | F | S | O | S | O | CH₃ |
| 1-182 | NO₂ | H | F | H | S | O | S | O | CH₃ |
| 1-183 | NO₂ | F | H | F | O | S | S | O | CH₃ |
| 1-184 | NO₂ | H | F | H | O | S | S | O | CH₃ |
| 1-185 | OH | F | H | F | O | O | O | S | H |
| 1-186 | OH | H | F | H | O | O | O | S | H |
| 1-187 | OH | F | H | F | S | O | O | S | H |
| 1-188 | OH | H | F | H | S | O | O | S | H |
| 1-189 | OH | F | H | F | O | S | O | S | H |
| 1-190 | OH | H | F | H | O | S | O | S | H |
| 1-191 | OH | F | H | F | O | O | S | O | H |
| 1-192 | OH | H | F | H | O | O | S | O | H |
| 1-193 | OH | F | H | F | S | O | S | O | H |
| 1-194 | OH | H | F | H | S | O | S | O | H |
| 1-195 | OH | F | H | F | O | S | S | O | H |
| 1-196 | OH | H | F | H | O | S | S | O | H |
| 1-197 | OH | F | H | F | O | O | O | S | CH₃ |
| 1-198 | OH | H | F | H | O | O | O | S | CH₃ |
| 1-199 | OH | F | H | F | S | O | O | S | CH₃ |
| 1-200 | OH | H | F | H | S | O | O | S | CH₃ |
| 1-201 | OH | F | H | F | O | S | O | S | CH₃ |
| 1-202 | OH | H | F | H | O | S | O | S | CH₃ |
| 1-203 | OH | F | H | F | O | O | S | O | CH₃ |
| 1-204 | OH | H | F | H | O | O | S | O | CH₃ |
| 1-205 | OH | F | H | F | S | O | S | O | CH₃ |
| 1-206 | OH | H | F | H | S | O | S | O | CH₃ |
| 1-207 | OH | F | H | F | O | S | S | O | CH₃ |
| 1-208 | OH | H | F | H | O | S | S | O | CH₃ |

TABLE 2
R⁵ is 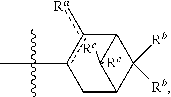 or a stereoisomer thereof

| | R¹ | R² | R³ | R⁴ | Y¹ | Z¹ | Z² | R^{a} | R^{b} | R^{c} |
|---|---|---|---|---|---|---|---|---|---|---|
| 2-1 | F | F | F | F | Bond | O | O | CH₃ | CH₃ | H |
| 2-2 | F | F | F | F | —CH₂— | O | O | CH₃ | CH₃ | H |
| 2-3 | F | F | F | F | —CH(CH₃)— | O | O | CH₃ | CH₃ | H |
| 2-4 | F | F | F | F | Bond | O | S | CH₃ | CH₃ | H |
| 2-5 | F | F | F | F | —CH₂— | O | S | CH₃ | CH₃ | H |
| 2-6 | F | F | F | F | —CH(CH₃)— | O | S | CH₃ | CH₃ | H |
| 2-7 | F | F | F | F | Bond | S | C | CH₃ | CH₃ | H |
| 2-8 | F | F | F | F | —CH₂— | S | O | CH₃ | CH₃ | H |
| 2-9 | F | F | F | F | —CH(CH₃)— | S | O | CH₃ | CH₃ | H |
| 2-10 | F | F | F | F | Bond | O | O | H | CH₃ | H |
| 2-11 | F | F | F | F | —CH₂— | O | C | H | CH₃ | H |
| 2-12 | F | F | F | F | —CH(CH₃)— | O | O | H | CH₃ | H |
| 2-13 | F | F | F | F | Bond | O | S | H | CH₃ | H |
| 2-14 | F | F | F | F | —CH₂— | O | S | H | CH₃ | H |
| 2-15 | F | F | F | F | —CH(CH₃)— | O | S | H | CH₃ | H |
| 2-16 | F | F | F | F | Bond | S | C | H | CH₃ | H |
| 2-17 | F | F | F | F | —CH₂— | S | C | H | CH₃ | H |
| 2-18 | F | F | F | F | —CH(CH₃)— | S | O | H | CH₃ | H |
| 2-19 | F | F | F | F | Bond | O | O | H | H | CH₃ |
| 2-20 | F | F | F | F | —CH₂— | O | C | H | H | CH₃ |
| 2-21 | F | F | F | F | —CH(CH₃)— | O | O | H | H | CH₃ |
| 2-22 | F | F | F | F | Bond | O | S | H | H | CH₃ |
| 2-23 | F | F | F | F | —CH₂— | O | S | H | H | CH₃ |
| 2-24 | F | F | F | F | —CH(CH₃)— | O | S | H | H | CH₃ |
| 2-25 | F | F | F | F | Bond | S | C | H | H | CH₃ |
| 2-26 | F | F | F | F | —CH₂— | S | O | H | H | CH₃ |
| 2-27 | F | F | F | F | —CH(CH₃)— | S | O | H | H | CH₃ |
| 2-28 | F | F | F | F | Bond | O | O | CH3 | H | CH₃ |
| 2-29 | F | F | F | F | —CH₂— | O | C | CH₃ | H | CH₃ |
| 2-30 | F | F | F | F | —CH(CH₃)— | O | O | CH₃ | H | CH₃ |
| 2-31 | F | F | F | F | Bond | O | S | CH₃ | H | CH₃ |
| 2-32 | F | F | F | F | —CH₂— | C | S | CH₃ | H | CH₃ |
| 2-33 | F | F | F | F | —CH(CH₃)— | O | S | CH₃ | H | CH₃ |
| 2-34 | F | F | F | F | Bond | S | O | CH₃ | H | CH₃ |
| 2-35 | F | F | F | F | —CH₂— | S | O | CH₃ | H | CH₃ |
| 2-36 | F | F | F | F | —CH(CH₃)— | S | C | CH₃ | H | CH₃ |
| 2-37 | F | F | F | F | Bond | O | O | CH₂OH | CH₃ | H |
| 2-38 | F | F | F | F | —CH₂— | O | C | CH₂OH | CH₃ | H |
| 2-39 | F | F | F | F | —CH(CH₃)— | O | O | CH₂OH | CH₃ | H |
| 2-40 | F | F | F | F | Bond | O | S | CH₂OH | CH₃ | H |
| 2-41 | F | F | F | F | —CH₂— | O | S | CH₂OH | CH | H |
| 2-42 | F | F | F | F | —CH(CH₃)— | O | S | CH₂OH | CH₃ | H |
| 2-43 | F | F | F | F | Bond | S | O | CH₂OH | CH₃ | H |
| 2-44 | F | F | F | F | —CH₂— | S | O | CH₂OH | CH₃ | H |
| 2-45 | F | F | F | F | —CH(CH₃)— | S | O | CH₂OH | CH₃ | H |
| 2-46 | F | F | F | F | Bond | O | O | ═CH₂ | CH₃ | H |
| 2-47 | F | F | F | F | —CH₂— | O | O | ═CH₂ | CH₃ | H |
| 2-48 | F | F | F | F | —CH(CH₃)— | O | O | ═CH₂ | CH₃ | H |
| 2-49 | F | F | F | F | Bond | O | S | ═CH₂ | CH₃ | H |
| 2-50 | F | F | F | F | —CH₂— | O | S | ═CH₂ | CH₃ | H |
| 2-51 | F | F | F | F | —CH(CH₃)— | O | S | ═CH₂ | CH₃ | H |
| 2-52 | F | F | F | F | Bond | S | O | ═CH₂ | CH₃ | H |
| 2-53 | F | F | F | F | —CH₂— | S | O | ═CH₂ | CH₃ | H |
| 2-54 | F | F | F | F | —CH(CH₃)— | S | O | ═CH₂ | CH₃ | H |
| 2-55 | F | H | H | F | Bond | O | O | CH₃ | CH₃ | H |
| 2-56 | F | H | H | F | —CH₂— | O | O | CH₃ | CH₃ | H |
| 2-57 | F | H | H | F | —CH(CH₃)— | O | O | CH₃ | CH₃ | H |
| 2-58 | F | H | H | F | Bond | O | S | CH₃ | CH₃ | H |
| 2-59 | F | H | H | F | —CH₂— | O | S | CH₃ | CH₃ | H |
| 2-60 | F | H | H | F | —CH(CH₃)— | O | S | CH₃ | CH₃ | H |
| 2-61 | F | H | H | F | Bond | S | O | CH₃ | CH₃ | H |
| 2-62 | F | H | H | F | —CH₂— | S | O | CH₃ | CH₃ | H |
| 2-63 | F | H | H | F | —CH(CH₃)— | S | O | CH₃ | CH₃ | H |
| 2-64 | F | H | H | F | Bond | O | O | H | CH₃ | H |
| 2-65 | F | H | H | F | —CH₂— | O | O | H | CH₃ | H |
| 2-66 | F | H | H | F | —CH(CH₃)— | O | O | H | CH₃ | H |
| 2-67 | F | H | H | F | Bond | O | S | H | CH₃ | H |
| 2-68 | F | H | H | F | —CH₂— | O | S | H | CH₃ | H |
| 2-69 | F | H | H | F | —CH(CH₃)— | O | S | H | CH₃ | H |
| 2-70 | F | H | H | F | Bond | S | O | H | CH₃ | H |
| 2-71 | F | H | H | F | —CH₂— | S | O | H | CH₃ | H |
| 2-72 | F | H | H | F | —CH(CH₃)— | S | O | H | CH₃ | H |
| 2-73 | F | H | H | F | Bond | O | O | H | H | CH₃ |
| 2-74 | F | H | H | F | —CH₂— | O | O | H | H | CH₃ |
| 2-75 | F | H | H | F | —CH(CH₃)— | O | O | H | H | CH₃ |
| 2-76 | F | H | H | F | Bond | O | S | H | H | CH₃ |
| 2-77 | F | H | H | F | —CH₂— | O | S | H | H | CH₃ |
| 2-78 | F | H | H | F | —CH(CH₃)— | O | S | H | H | CH₃ |
| 2-79 | F | H | H | F | Bond | S | O | H | H | CH₃ |
| 2-80 | F | H | H | F | —CH₂— | S | O | H | H | CH₃ |
| 2-81 | F | H | H | F | —CH(CH₃)— | S | O | H | H | CH₃ |
| 2-82 | F | H | H | F | Bond | O | O | CH₃ | H | CH₃ |
| 2-83 | F | H | H | F | —CH₂— | O | O | CH₃ | H | CH₃ |
| 2-84 | F | H | H | F | —CH(CH₃)— | O | C | CH₃ | H | CH₃ |
| 2-85 | F | H | H | F | Bond | O | S | CH₃ | H | CH₃ |
| 2-86 | F | H | H | F | —CH₂— | O | S | CH₃ | H | CH₃ |
| 2-87 | F | H | H | F | —CH(CH₃)— | O | S | CH₃ | H | CH₃ |
| 2-88 | F | H | H | F | Bond | S | O | CH₃ | H | CH₃ |
| 2-89 | F | H | H | F | —CH₂— | S | C | CH₃ | H | CH₃ |
| 2-90 | F | H | H | F | —CH(CH₃)— | S | O | CH₃ | H | CH₃ |
| 2-91 | F | H | H | F | Bond | O | O | CH₂OH | CH₃ | H |
| 2-92 | F | H | H | F | —CH₂— | O | C | CH₂OH | CH₃ | H |
| 2-93 | F | H | H | F | —CH(CH₃)— | O | O | CH₂OH | CH₃ | H |
| 2-94 | F | H | H | F | Bond | O | S | CH₂OH | CH₃ | H |
| 2-95 | F | H | H | F | —CH₂— | O | S | CH₂OH | CH₃ | H |
| 2-96 | F | H | H | F | —CH(CH₃)— | O | S | CH₂OH | CH₃ | H |
| 2-97 | F | H | H | F | Bond | S | C | CH₂OH | CH₃ | H |
| 2-98 | F | H | H | F | —CH₂— | S | O | CH₂OH | CH₃ | H |
| 2-99 | F | H | H | F | —CH(CH₃)— | S | C | CH₂OH | CH₃ | H |
| 2-100 | F | H | H | F | Bond | O | O | ═CH₂ | CH₃ | H |
| 2-101 | F | H | H | F | —CH₂— | O | O | ═CH₂ | CH₃ | H |
| 2-102 | F | H | H | F | —CH(CH₃)— | O | O | ═CH₂ | CH₃ | H |
| 2-103 | F | H | H | F | Bond | O | S | ═CH₂ | CH₃ | H |
| 2-104 | F | H | H | F | —CH₂— | O | S | ═CH₂ | CH₃ | H |
| 2-105 | F | H | H | F | —CH(CH₃)— | O | S | ═CH₂ | CH₃ | H |
| 2-106 | F | H | H | F | Bond | S | O | ═CH₂ | CH₃ | H |
| 2-107 | F | H | H | F | —CH₂— | S | O | ═CH₂ | CH₃ | H |
| 2-108 | F | H | H | F | —CH(CH₃)— | S | O | ═CH₂ | CH₃ | H |

TABLE 3
R⁵ is 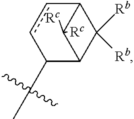 or a stereoisomer thereof

| | R¹ | R² | R³ | R⁴ | Y¹ | Z¹ | Z² | R^{b} | R^{c} |
|---|---|---|---|---|---|---|---|---|---|
| 3-1 | F | F | F | F | Bond | O | O | CH₃ | H |
| 3-2 | F | F | F | F | —CH₂— | O | O | CH₃ | H |
| 3-3 | F | F | F | F | —CH(CH₃)— | O | O | CH₃ | H |
| 3-4 | F | F | F | F | Bond | O | S | CH₃ | H |
| 3-5 | F | F | F | F | —CH₂— | O | S | CH₃ | H |
| 3-6 | F | F | F | F | —CH(CH₃)— | O | S | CH₃ | H |
| 3-7 | F | F | F | F | Bond | S | O | CH₃ | H |
| 3-8 | F | F | F | F | —CH₂— | S | O | CH₃ | H |
| 3-9 | F | F | F | F | —CH(CH₃)— | S | O | CH₃ | H |
| 3-10 | F | F | F | F | Bond | O | O | H | CH₃ |
| 3-11 | F | F | F | F | —CH₂— | O | O | H | CH₃ |
| 3-12 | F | F | F | F | —CH(CH₃)— | O | O | H | CH₃ |
| 3-13 | F | F | F | F | Bond | O | S | H | CH₃ |
| 3-14 | F | F | F | F | —CH₂— | O | S | H | CH₃ |
| 3-15 | F | F | F | F | —CH(CH₃)— | O | S | H | CH₃ |
| 3-16 | F | F | F | F | Bond | S | O | H | CH₃ |
| 3-17 | F | F | F | F | —CH₂— | S | O | H | CH₃ |
| 3-18 | F | F | F | F | —CH(CH₃)— | S | O | H | CH₃ |
| 3-19 | F | H | H | F | Bond | O | O | CH₃ | H |
| 3-20 | F | H | H | F | —CH₂— | O | O | CH₃ | H |
| 3-21 | F | H | H | F | —CH(CH₃)— | O | O | CH₃ | H |
| 3-22 | F | H | H | F | Bond | O | S | CH₃ | H |
| 3-23 | F | H | H | F | —CH₂— | O | S | CH₃ | H |
| 3-24 | F | H | H | F | —CH(CH₃)— | O | S | CH₃ | H |
| 3-25 | F | H | H | F | Bond | S | O | CH₃ | H |
| 3-26 | F | H | H | F | —CH₂— | S | O | CH₃ | H |
| 3-27 | F | H | H | F | —CH(CH₃)— | S | O | CH₃ | H |
| 3-28 | F | H | H | F | Bond | O | O | H | CH₃ |
| 3-29 | F | H | H | F | —CH₂— | O | O | H | CH₃ |
| 3-30 | F | H | H | F | —CH(CH₃)— | O | O | H | CH₃ |
| 3-31 | F | H | H | F | Bond | O | S | H | CH₃ |
| 3-32 | F | H | H | F | —CH₂— | O | S | H | CH₃ |
| 3-33 | F | H | H | F | —CH(CH₃)— | O | S | H | CH₃ |
| 3-34 | F | H | H | F | Bond | S | O | H | CH₃ |
| 3-35 | F | H | H | F | —CH₂— | S | O | H | CH₃ |
| 3-36 | F | H | H | F | —CH(CH₃)— | S | O | H | CH₃ |

TABLE 4
R⁵ is 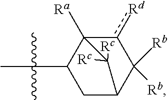 or a stereoisomer thereof

| | R¹ | R² | R³ | R⁴ | Y¹ | Z¹ | Z² | R^{a} | R^{b} | R^{c} | R^{d} |
|---|---|---|---|---|---|---|---|---|---|---|---|
| 4-1 | F | F | F | F | Bond | O | O | CH₃ | CH₃ | H | H |
| 4-2 | F | F | F | F | —CH₂— | O | O | CH₃ | CH₃ | H | H |
| 4-3 | F | F | F | F | —CH(CH₃)— | O | O | CH₃ | CH₃ | H | H |
| 4-4 | F | F | F | F | Bond | O | S | CH₃ | CH₃ | H | H |
| 4-5 | F | F | F | F | —CH₂— | O | S | CH₃ | CH₃ | H | H |
| 4-6 | F | F | F | F | —CH(CH₃)— | O | S | CH₃ | CH₃ | H | H |
| 4-7 | F | F | F | F | Bond | S | O | CH₃ | CH₃ | H | H |
| 4-8 | F | F | F | F | —CH₂— | S | O | CH₃ | CH₃ | H | H |
| 4-9 | F | F | F | F | —CH(CH₃)— | S | O | CH₃ | CH₃ | H | H |
| 4-10 | F | F | F | F | Bond | O | O | H | CH₃ | H | H |
| 4-11 | F | F | F | F | —CH₂— | O | O | H | CH₃ | H | H |
| 4-12 | F | F | F | F | —CH(CH₃)— | O | O | H | CH₃ | H | H |
| 4-13 | F | F | F | F | Bond | O | S | H | CH₃ | H | H |
| 4-14 | F | F | F | F | —CH₂— | O | S | H | CH₃ | H | H |
| 4-15 | F | F | \|F | F | —CH(CH₃)— | O | S | H | CH₃ | H | H |
| 4-16 | F | F | F | F | Bond | S | O | H | CH₃ | H | H |
| 4-17 | F | F | F | F | —CH₂— | S | O | H | CH₃ | H | H |
| 4-18 | F | F | F | F | —CH(CH₃)— | S | O | H | CH₃ | H | H |
| 4-19 | F | F | F | F | Bond | O | O | H | H | CH₃ | H |
| 4-20 | F | F | F | F | —CH₂— | O | O | H | H | CH₃ | H |
| 4-21 | F | F | F | F | —CH(CH₃)— | O | O | H | H | CH₃ | H |
| 4-22 | F | F | F | F | Bond | O | S | H | H | CH₃ | H |
| 4-23 | F | F | F | F | —CH₂— | O | S | H | H | CH₃ | H |
| 4-24 | F | F | F | F | —CH(CH₃)— | O | S | H | H | CH₃ | H |
| 4-25 | F | F | F | F | Bond | S | O | H | H | CH₃ | H |
| 4-26 | F | F | F | F | —CH₂— | S | O | H | H | CH₃ | H |
| 4-27 | F | F | F | F | —CH(CH₃)— | S | O | H | H | CH₃ | H |
| 4-28 | F | F | F | F | Bond | O | O | CH₃ | H | CH₃ | H |
| 4-29 | F | F | F | F | —CH₂— | O | O | CH₃ | H | CH₃ | H |
| 4-30 | F | F | F | F | —CH(CH₃)— | O | O | CH₃ | H | CH₃ | H |
| 4-31 | F | F | F | F | Bond | O | S | CH₃ | H | CH₃ | H |
| 4-32 | F | F | F | F | —CH₂— | O | S | CH₃ | H | CH₃ | H |
| 4-33 | F | F | F | F | —CH(CH₃)— | O | S | CH₃ | H | CH₃ | H |
| 4-34 | F | F | F | F | Bond | S | O | CH₃ | H | CH₃ | H |
| 4-35 | F | F | F | F | —CH₂— | S | O | CH₃ | H | CH₃ | H |
| 4-36 | F | F | F | F | —CH(CH₃)— | S | O | CH₃ | H | CH₃ | H |
| 4-37 | F | F | F | F | Bond | O | O | CH₂OH | H | CH₃ | H |
| 4-38 | F | F | F | F | —CH₂— | O | O | CH₂OH | H | CH₃ | H |
| 4-39 | F | F | F | F | —CH(CH₃)— | O | O | CH₂OH | H | CH₃ | H |
| 4-40 | F | F | F | F | Bond | O | S | CH₂OH | H | CH₃ | H |
| 4-41 | F | F | F | F | —CH₂— | O | S | CH₂OH | H | CH₃ | H |
| 4-42 | F | F | F | F | —CH(CH₃)— | O | S | CH₂OH | H | CH₃ | H |
| 4-43 | F | F | F | F | Bond | S | O | CH₂OH | H | CH₃ | H |
| 4-44 | F | F | F | F | —CH₂— | S | O | CH₂OH | H | CH₃ | H |
| 4-45 | F | F | F | F | —CH(CH₃)— | S | O | CH₂OH | H | CH₃ | H |
| 4-46 | F | F | F | F | Bond | O | O | CH₃ | CH₃ | H | OH |
| 4-47 | F | F | F | F | —CH₂— | O | O | CH₃ | CH₃ | H | OH |
| 4-48 | F | F | F | F | —CH(CH₃)— | O | O | CH₃ | CH₃ | H | OH |
| 4-49 | F | F | F | F | Bond | O | S | CH₃ | CH₃ | H | OH |
| 4-50 | F | F | F | F | —CH₂— | O | S | CH₃ | CH₃ | H | OH |
| 4-51 | F | F | F | F | —CH(CH₃)— | O | S | CH₃ | CH₃ | H | OH |
| 4-52 | F | F | F | F | Bond | S | O | CH₃ | CH₃ | H | OH |
| 4-53 | F | F | F | F | —CH₂— | S | O | CH₃ | CH₃ | H | OH |
| 4-54 | F | F | F | F | —CH(CH₃)— | S | O | CH₃ | CH₃ | H | OH |
| 4-55 | F | F | F | F | Bond | O | O | H | CH₃ | H | OH |
| 4-56 | F | F | F | F | —CH₂— | O | O | H | CH₃ | H | OH |
| 4-57 | F | F | F | F | —CH(CH₃)— | O | O | H | CH₃ | H | OH |
| 4-58 | F | F | F | F | Bond | O | S | H | CH₃ | H | OH |
| 4-59 | F | F | F | F | —CH₂— | O | S | H | CH₃ | H | OH |
| 4-60 | F | F | F | F | —CH(CH₃)— | O | S | H | CH₃ | H | OH |
| 4-61 | F | F | F | F | Bond | S | O | H | CH₃ | H | OH |
| 4-62 | F | F | F | F | —CH₂— | S | O | H | CH₃ | H | OH |
| 4-63 | F | F | F | F | —CH(CH₃)— | S | O | H | CH₃ | H | OH |
| 4-64 | F | F | F | F | Bond | O | O | H | H | CH₃ | OH |
| 4-65 | F | F | F | F | —CH₂— | O | O | H | H | CH₃ | OH |
| 4-66 | F | F | F | F | —CH(CH₃)— | O | O | H | H | CH₃ | OH |
| 4-67 | F | F | F | F | Bond | O | S | H | H | CH₃ | OH |
| 4-68 | F | F | F | F | —CH₂— | O | S | H | H | CH₃ | OH |
| 4-69 | F | F | F | F | —CH(CH₃)— | O | S | H | H | CH₃ | OH |
| 4-70 | F | F | F | F | Bond | S | O | H | H | CH₃ | OH |
| 4-72 | F | F | F | F | —CH₂— | S | O | H | H | CH₃ | OH |
| 4-72 | F | F | F | F | —CH(CH₃)— | S | O | H | H | CH₃ | OH |
| 4-73 | F | F | F | F | Bond | O | O | CH₃ | H | CH₃ | OH |
| 4-74 | F | F | F | F | —CH₂— | O | O | CH₃ | H | CH₃ | OH |
| 4-75 | F | F | F | F | —CH(CH₃)— | O | O | CH₃ | H | CH₃ | OH |
| 4-76 | F | F | F | F | Bond | O | S | CH₃ | H | CH₃ | OH |
| 4-77 | F | F | F | F | —CH₂— | O | S | CH₃ | H | CH₃ | OH |
| 4-78 | F | F | F | F | —CH(CH₃)— | O | S | CH₃ | H | CH₃ | OH |
| 4-79 | F | F | F | F | Bond | S | O | CH₃ | H | CH₃ | OH |
| 4-80 | F | F | F | F | —CH₂— | S | O | CH₃ | H | CH₃ | OH |
| 4-81 | F | F | F | F | —CH(CH₃)— | S | O | CH₃ | H | CH₃ | OH |
| 4-82 | F | F | F | F | Bond | O | O | CH₂OH | H | CH₃ | OH |
| 4-83 | F | F | F | F | —CH₂— | O | O | CH₂OH | H | CH₃ | OH |
| 4-84 | F | F | F | F | —CH(CH₃)— | O | O | CH₂OH | H | CH₃ | OH |
| 4-85 | F | F | F | F | Bond | O | S | CH₂OH | H | CH₃ | OH |
| 4-86 | F | F | F | F | —CH₂— | O | S | CH₂OH | H | CH₃ | OH |
| 4-87 | F | F | F | F | —CH(CH₃)— | O | S | CH₂OH | H | CH₃ | OH |
| 4-88 | F | F | F | F | Bond | S | O | CH₂OH | H | CH₃ | OH |
| 4-89 | F | F | F | F | —CH₂— | S | O | CH₂OH | H | CH₃ | OH |
| 4-90 | F | F | F | F | —CH(CH₃)— | S | O | CH₂OH | H | CH₃ | OH |
| 4-91 | F | F | F | F | Bond | O | O | CH₃ | CH₃ | H | ═O |
| 4-92 | F | F | F | F | —CH₂— | O | O | CH₃ | CH₃ | H | ═O |
| 4-93 | F | F | F | F | —CH(CH₃)— | O | O | CH₃ | CH₃ | H | ═O |
| 4-94 | F | F | F | F | Bond | O | S | CH₃ | CH₃ | H | ═O |
| 4-95 | F | F | F | F | —CH₂— | O | S | CH₃ | CH₃ | H | ═O |
| 4-96 | F | F | F | F | —CH(CH₃)— | O | S | CH₃ | CH₃ | H | ═O |
| 4-97 | F | F | F | F | Bond | S | O | CH₃ | CH₃ | H | ═O |
| 4-98 | F | F | F | F | —CH₂— | S | O | CH₃ | CH₃ | H | ═O |
| 4-99 | F | F | F | F | —CH(CH₃)— | S | O | CH₃ | CH₃ | H | ═O |
| 4-100 | F | F | F | F | Bond | O | O | H | CH₃ | H | ═O |
| 4-101 | F | F | F | F | —CH₂— | O | O | H | CH₃ | H | ═O |
| 4-102 | F | F | F | F | —CH(CH₃)— | O | O | H | CH₃ | H | ═O |
| 4-103 | F | F | F | F | Bond | O | S | H | CH₃ | H | ═O |
| 4-104 | F | F | F | F | —CH₂— | O | S | H | CH₃ | H | ═O |
| 4-105 | F | F | F | F | —CH(CH₃)— | O | S | H | CH₃ | H | ═O |
| 4-106 | F | F | F | F | Bond | S | O | H | CH₃ | H | ═O |
| 4-107 | F | F | F | F | —CH₂— | S | O | H | CH₃ | H | ═O |
| 4-108 | F | F | F | F | —CH(CH₃)— | S | O | H | CH₃ | H | ═O |
| 4-109 | F | F | F | F | Bond | O | O | H | H | CH₃ | ═O |
| 4-110 | F | F | F | F | —CH₂— | O | O | H | H | CH₃ | ═O |
| 4-111 | F | F | F | F | —CH(CH₃)— | O | O | H | H | CH₃ | ═O |
| 4-112 | F | F | F | F | Bond | O | S | H | H | CH₃ | ═O |
| 4-113 | F | F | F | F | —CH₂— | O | S | H | H | CH₃ | ═O |
| 4-114 | F | F | F | F | —CH(CH₃)— | O | S | H | H | CH₃ | ═O |
| 4-115 | F | F | F | F | Bond | S | O | H | H | CH₃ | ═O |
| 4-116 | F | F | F | F | —CH₂— | S | O | H | H | CH₃ | ═O |
| 4-117 | F | F | F | F | —CH(CH₃)— | S | O | H | H | CH₃ | ═O |
| 4-118 | F | F | F | F | Bond | O | O | CH₃ | H | CH₃ | ═O |
| 4-119 | F | F | F | F | —CH₂— | O | O | CH₃ | H | CH₃ | ═O |
| 4-120 | F | F | F | F | —CH(CH₃)— | O | O | CH₃ | H | CH₃ | ═O |
| 4-121 | F | F | F | F | Bond | O | S | CH₃ | H | CH₃ | ═O |
| 4-122 | F | F | F | F | —CH₂— | O | S | CH₃ | H | CH₃ | ═O |
| 4-123 | F | F | F | F | —CH(CH₃)— | O | S | CH₃ | H | CH₃ | ═O |
| 4-124 | F | F | F | F | Bond | S | O | CH₃ | H | CH₃ | ═O |
| 4-125 | F | F | F | F | —CH₂— | S | O | CH₃ | H | CH₃ | ═O |
| 4-126 | F | F | F | F | —CH(CH₃)— | S | O | CH₃ | H | CH₃ | ═O |
| 4-127 | F | F | F | F | Bond | O | O | CH₂OH | H | CH₃ | ═O |
| 4-128 | F | F | F | F | —CH₂— | O | O | CH₂OH | H | CH₃ | ═O |
| 4-129 | F | F | F | F | —CH(CH₃)— | O | O | CH₂OH | H | CH | ═O |
| 4-130 | F | F | F | F | Bond | O | S | CH₂OH | H | CH₃ | ═O |
| 4-131 | F | F | F | F | —CH₂— | O | S | CH₂OH | H | CH₃ | ═O |
| 4-132 | F | F | F | F | —CH(CH₃)— | O | S | CH₂OH | H | CH₃ | ═O |
| 4-133 | F | F | F | F | Bond | S | O | CH₂OH | H | CH₃ | ═O |
| 4-134 | F | F | F | F | —CH₂— | S | O | CH₂OH | H | CH₃ | ═O |
| 4-135 | F | F | F | F | —CH(CH₃)— | S | O | CH₂OH | H | CH₃ | ═O |
| 4-136 | F | H | H | F | Bond | O | O | CH₃ | CH₃ | H | H |
| 4-137 | F | H | H | F | —CH₂— | O | O | CH₃ | CH₃ | H | H |
| 4-138 | F | H | H | F | —CH(CH₃)— | O | O | CH₃ | CH₃ | H | H |
| 4-139 | F | H | H | F | Bond | O | S | CH₃ | CH₃ | H | H |
| 4-140 | F | H | H | F | —CH₂— | O | S | CH₃ | CH₃ | H | H |
| 4-141 | F | H | H | F | —CH(CH₃)— | O | S | CH₃ | CH₃ | H | H |
| 4-142 | F | H | H | F | Bond | S | O | CH₃ | CH₃ | H | H |
| 4-143 | F | H | H | F | —CH₂— | S | O | CH₃ | CH₃ | H | H |
| 4-144 | F | H | H | F | —CH(CH₃)— | S | O | CH₃ | CH₃ | H | H |
| 4-145 | F | H | H | F | Bond | O | O | H | CH₃ | H | H |
| 4-146 | F | H | H | F | —CH₂— | O | O | H | CH₃ | H | H |
| 4-147 | F | H | H | F | —CH(CH₃)— | O | O | H | CH₃ | H | H |
| 4-148 | F | H | H | F | Bond | O | S | H | CH₃ | H | H |
| 4-149 | F | H | H | F | —CH₂— | O | S | H | CH₃ | H | H |
| 4-150 | F | H | H | F | —CH(CH₃)— | O | S | H | CH₃ | H | H |
| 4-151 | F | H | H | F | Bond | S | O | H | CH₃ | H | H |
| 4-152 | F | H | H | F | —CH₂— | S | O | H | CH₃ | H | H |
| 4-153 | F | H | H | F | —CH(CH₃)— | S | O | H | CH₃ | H | H |
| 4-154 | F | H | H | F | Bond | O | O | H | H | CH₃ | H |
| 4-155 | F | H | H | F | —CH₂— | O | O | H | H | CH₃ | H |
| 4-156 | F | H | H | F | —CH(CH₃)— | O | O | H | H | CH₃ | H |
| 4-157 | F | H | H | F | Bond | O | S | H | H | CH₃ | H |
| 4-158 | F | H | H | F | —CH₂— | O | S | H | H | CH₃ | H |
| 4-159 | F | H | H | F | —CH(CH₃)— | O | S | H | H | CH₃ | H |
| 4-160 | F | H | H | F | Bond | S | O | H | H | CH₃ | H |
| 4-161 | F | H | H | F | —CH₂— | S | O | H | H | CH₃ | H |
| 4-162 | F | H | H | F | —CH(CH₃)— | S | O | H | H | CH₃ | H |
| 4-163 | F | H | H | F | Bond | O | O | CH₃ | H | CH₃ | H |
| 4-164 | F | H | H | F | —CH₂— | O | O | CH₃ | H | CH₃ | H |
| 4-165 | F | H | H | F | —CH(CH₃)— | O | O | CH₃ | H | CH₃ | H |
| 4-166 | F | H | H | F | Bond | O | S | CH₃ | H | CH₃ | H |
| 4-167 | F | H | H | F | —CH₂— | O | S | CH₃ | H | CH₃ | H |
| 4-168 | F | H | H | F | —CH(CH₃)— | O | S | CH₃ | H | CH₃ | H |
| 4-169 | F | H | H | F | Bond | S | O | CH₃ | H | CH₃ | H |
| 4-170 | F | H | H | F | —CH₂— | S | O | CH₃ | H | CH₃ | H |
| 4-171 | F | H | H | F | —CH(CH₃)— | S | O | CH₃ | H | CH₃ | H |
| 4-172 | F | H | H | F | Bond | O | O | CH₂OH | H | CH | H |
| 4-173 | F | H | H | F | —CH₂— | O | O | CH₂OH | H | CH₃ | H |
| 4-174 | F | H | H | F | —CH(CH₃)— | O | O | CH₂OH | H | CH₃ | H |
| 4-175 | F | H | H | F | Bond | O | S | CH₂OH | H | CH₃ | H |
| 4-176 | F | H | H | F | —CH₂— | O | S | CH₂OH | H | CH₃ | H |
| 4-177 | F | H | H | F | —CH(CH₃)— | O | S | CH₂OH | H | CH₃ | H |
| 4-178 | F | H | H | F | Bond | S | O | CH₂OH | H | CH₃ | H |
| 4-179 | F | H | H | F | —CH₂— | S | O | CH₂OH | H | CH₃ | H |
| 4-180 | F | H | H | F | —CH(CH₃)— | S | O | CH₂OH | H | CH₃ | H |
| 4-181 | F | H | H | F | Bond | O | O | CH₃ | CH₃ | H | OH |
| 4-182 | F | H | H | F | —CH₂— | O | O | CH₃ | CH₃ | H | OH |
| 4-183 | F | H | H | F | —CH(CH₃)— | O | O | CH₃ | CH₃ | H | OH |
| 4-184 | F | H | H | F | Bond | O | S | CH₃ | CH₃ | H | OH |
| 4-185 | F | H | H | F | —CH₂— | O | S | CH₃ | CH₃ | H | OH |
| 4-186 | F | H | H | F | —CH(CH₃)— | O | S | CH₃ | CH₃ | H | OH |
| 4-187 | F | H | H | F | Bond | S | O | CH₃ | CH₃ | H | OH |
| 4-188 | F | H | H | F | —CH₂— | S | O | CH₃ | CH₃ | H | OH |
| 4-189 | F | H | H | F | —CH(CH₃)— | S | O | CH₃ | CH₃ | H | OH |
| 4-190 | F | H | H | F | Bond | O | O | H | CH₃ | H | OH |
| 4-191 | F | H | H | F | —CH₂— | O | O | H | CH₃ | H | OH |
| 4-192 | F | H | H | F | —CH(CH₃)— | O | O | H | CH₃ | H | OH |
| 4-193 | F | H | H | F | Bond | O | S | H | CH₃ | H | OH |
| 4-194 | F | H | H | F | —CH₂— | O | S | H | CH₃ | H | OH |
| 4-195 | F | H | H | F | —CH(CH₃)— | O | S | H | CH₃ | H | OH |
| 4-196 | F | H | H | F | Bond | S | O | H | CH₃ | H | OH |
| 4-197 | F | H | H | F | —CH₂— | S | O | H | CH₃ | H | OH |
| 4-198 | F | H | H | F | —CH(CH₃)— | S | O | H | CH₃ | H | OH |
| 4-199 | F | H | H | F | Bond | O | O | H | H | CH₃ | OH |
| 4-200 | F | H | H | F | —CH₂— | O | O | H | H | CH₃ | OH |
| 4-201 | F | H | H | F | —CH(CH₃)— | O | O | H | H | CH₃ | OH |
| 4-202 | F | H | H | F | Bond | O | S | H | H | CH₃ | OH |
| 4-203 | F | H | H | F | —CH₂— | O | S | H | H | CH₃ | OH |
| 4-204 | F | H | H | F | —CH(CH₃)— | O | S | H | H | CH₃ | OH |
| 4-205 | F | H | H | F | Bond | S | O | H | H | CH₃ | OH |
| 4-206 | F | H | H | F | —CH₂— | S | O | H | H | CH₃ | OH |
| 4-207 | F | H | H | F | —CH(CH₃)— | S | O | H | H | CH₃ | OH |
| 4-208 | F | H | H | F | Bond | O | O | CH₃ | H | CH₃ | OH |
| 4-209 | F | H | H | F | —CH₂— | O | O | CH₃ | H | CH₃ | OH |
| 4-210 | F | H | H | F | —CH(CH₃)— | O | O | CH₃ | H | CH₃ | OH |
| 4-211 | F | H | H | F | Bond | O | S | CH₃ | H | CH₃ | OH |
| 4-212 | F | H | H | F | —CH₂— | O | S | CH₃ | H | CH₃ | OH |
| 4-213 | F | H | H | F | —CH(CH₃)— | O | S | CH₃ | H | CH₃ | OH |
| 4-214 | F | H | H | F | Bond | S | O | CH₃ | H | CH₃ | OH |
| 4-215 | F | H | H | F | —CH₂— | S | O | CH₃ | H | CH₃ | OH |
| 4-216 | F | H | H | F | —CH(CH₃)— | S | C | CH₃ | H | CH₃ | OH |
| 4-217 | F | H | H | F | Bond | O | O | CH₂OH | H | CH₃ | OH |
| 4-218 | F | H | H | F | —CH₂— | O | O | CH₂OH | H | CH₃ | OH |
| 4-219 | F | H | H | F | —CH(CH₃)— | O | O | CH₂OH | H | CH₃ | OH |
| 4-220 | F | H | H | F | Bond | O | S | CH₂OH | H | CH₃ | OH |
| 4-221 | F | H | H | F | —CH₂— | O | S | CH₂OH | H | CH₃ | OH |
| 4-222 | F | H | H | F | —CH(CH₃)— | O | S | CH₂OH | H | CH₃ | OH |
| 4-223 | F | H | H | F | Bond | S | C | CH₂OH | H | CH₃ | OH |
| 4-224 | F | H | H | F | —CH₂— | S | O | CH₂OH | H | CH₃ | OH |
| 4-225 | F | H | H | F | —CH(CH₃)— | S | O | CH₂OH | H | CH₃ | OH |
| 4-226 | F | H | H | F | Bond | 0 | O | CH₃ | CH₃ | H | ═O |
| 4-227 | F | H | H | F | —CH₂— | O | C | CH₃ | CH₃ | H | ═O |
| 4-228 | F | H | H | F | —CH(CH₃)— | O | O | CH₃ | CH₃ | H | ═O |
| 4-229 | F | H | H | F | Bond | O | S | CH₃ | CH₃ | H | ═O |
| 4-230 | F | H | H | F | —CH₂— | O | S | CH₃ | CH₃ | H | ═O |
| 4-231 | F | H | H | F | —CH(CH₃)— | O | S | CH₃ | CH₃ | H | ═O |
| 4-232 | F | H | H | F | Bond | S | O | CH₃ | CH₃ | H | ═O |
| 4-233 | F | H | H | F | —CH₂— | S | O | CH₃ | CH₃ | H | ═O |
| 4-234 | F | H | H | F | —CH(CH₃)— | S | O | CH₃ | CH₃ | H | ═O |
| 4-235 | F | H | H | F | Bond | O | O | H | CH₃ | H | ═O |
| 4-236 | F | H | H | F | —CH₂— | O | O | H | CH₃ | H | ═O |
| 4-237 | F | H | H | F | —CH(CH₃)— | O | O | H | CH₃ | H | ═O |
| 4-238 | F | H | H | F | Bond | O | S | H | CH₃ | H | ═O |
| 4-239 | F | H | H | F | —CH₂— | O | S | H | CH₃ | H | ═O |
| 4-240 | F | H | H | F | —CH(CH₃)— | O | S | H | CH₃ | H | ═O |
| 4-241 | F | H | H | F | Bond | S | O | H | CH₃ | H | ═O |
| 4-242 | F | H | H | F | —CH₂— | S | O | H | CH₃ | H | ═O |
| 4-243 | F | H | H | F | —CH(CH₃)— | S | O | H | CH₃ | H | ═O |
| 4-244 | F | H | H | F | Bond | O | O | H | H | CH₃ | ═O |
| 4-245 | F | H | H | F | —CH₂— | O | O | H | H | CH₃ | ═O |
| 4-246 | F | H | H | F | —CH(CH₃)— | O | O | H | H | CH₃ | ═O |
| 4-247 | F | H | H | F | Bond | O | S | H | H | CH₃ | ═O |
| 4-248 | F | H | H | F | —CH₂— | O | S | H | H | CH₃ | ═O |
| 4-249 | F | H | H | F | —CH(CH₃)— | O | S | H | H | CH₃ | ═O |
| 4-250 | F | H | H | F | Bond | S | O | H | H | CH₃ | ═O |
| 4-251 | F | H | H | F | —CH₂— | S | O | H | H | CH₃ | ═O |
| 4-252 | F | H | H | F | —CH(CH₃)— | S | O | H | H | CH₃ | ═O |
| 4-253 | F | H | H | F | Bond | O | O | CH₃ | H | CH₃ | ═O |
| 4-254 | F | H | H | F | —CH₂— | O | O | CH₃ | H | CH₃ | ═O |
| 4-255 | F | H | H | F | —CH(CH₃)— | O | O | CH₃ | H | CH₃ | ═O |
| 4-256 | F | H | H | F | Bond | O | S | CH₃ | H | CH₃ | ═O |
| 4-257 | F | H | H | F | —CH₂— | O | S | CH₃ | H | CH₃ | ═O |
| 4-258 | F | H | H | F | —CH(CH₃)— | O | S | CH₃ | H | CH₃ | ═O |
| 4-259 | F | H | H | F | Bond | S | O | CH₃ | H | CH₃ | ═O |
| 4-260 | F | H | H | F | —CH₂— | S | O | CH₃ | H | CH₃ | ═O |
| 4-261 | F | H | H | F | —CH(CH₃)— | S | O | CH₃ | H | CH₃ | ═O |
| 4-262 | F | H | H | F | Bond | O | O | CH₂OH | H | CH₃ | ═O |
| 4-263 | F | H | H | F | —CH₂— | O | O | CH₂OH | H | CH₃ | ═O |
| 4-264 | F | H | H | F | —CH(CH₃)— | O | O | CH₂OH | H | CH₃ | ═O |
| 4-265 | F | H | H | F | Bond | O | S | CH₂OH | H | CH₃ | ═O |
| 4-266 | F | H | H | F | —CH₂— | O | S | CH₂OH | H | CH₃ | ═O |
| 4-267 | F | H | H | F | —CH(CH₃)— | O | S | CH₂OH | H | CH₃ | ═O |
| 4-268 | F | H | H | F | Bond | S | O | CH₂OH | H | CH₃ | ═O |
| 4-269 | F | H | H | F | —CH₂— | S | O | CH₂OH | H | CH₃ | ═O |
| 4-270 | F | H | H | F | —CH(CH₃)— | S | O | CH₂OH | H | CH₃ | ═O |

Several exemplary compounds are shown in Table 5.

TABLE 5

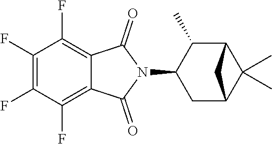 [SMILES: C[C@H]1[C@H](N2C(=O)c3c(F)c(F)c(F)c(F)c3C2=O)C[C@@H]2C[C@H]1C2(C)C]

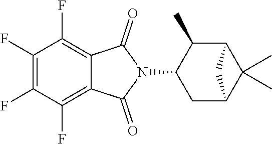 [SMILES: C[C@@H]1[C@@H](N2C(=O)c3c(F)c(F)c(F)c(F)c3C2=O)C[C@H]2C[C@@H]1C2(C)C]

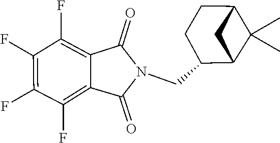 [SMILES: CC1(C)[C@H]2CC[C@@H](CN3C(=O)c4c(F)c(F)c(F)c(F)c4C3=O)[C@@H]1C2]

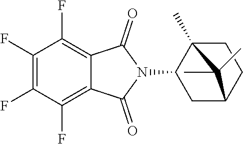 [SMILES: CC1(C)[C@@H]2CC[C@]1(C)[C@@H](N1C(=O)c3c(F)c(F)c(F)c(F)c3C1=O)C2]

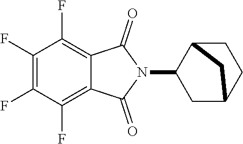

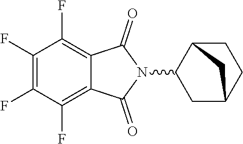 [SMILES: O=C1c2c(F)c(F)c(F)c(F)c2C(=O)N1C1C[C@H]2CC[C@@H]1C2]

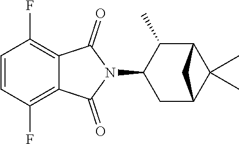 [SMILES: C[C@H]1[C@H](N2C(=O)c3c(F)ccc(F)c3C2=O)C[C@@H]2C[C@H]1C2(C)C]

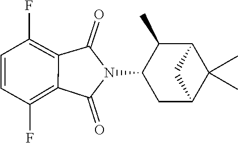 [SMILES: C[C@@H]1[C@@H](N2C(=O)c3c(F)ccc(F)c3C2=O)C[C@H]2C[C@@H]1C2(C)C]

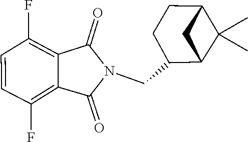

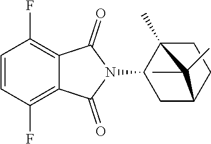 [SMILES: CC1(C)[C@@H]2CC[C@]1(C)[C@@H](N1C(=O)c3c(F)ccc(F)c3C1=O)C2]

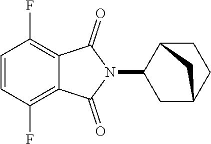

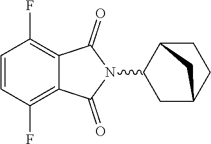 [SMILES: O=C1c2c(F)ccc(F)c2C(=O)N1C1C[C@H]2CC[C@@H]1C2]

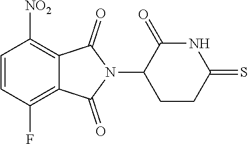 [SMILES: O=C1NC(=S)CCC1N1C(=O)c2c(F)ccc([N+](=O)[O-])c2C1=O]

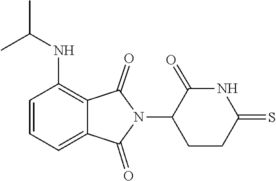 [SMILES: CC(C)Nc1cccc2c1C(=O)N(C1CCC(=S)NC1=O)C2=O]

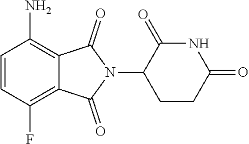 [SMILES: Nc1ccc(F)c2c1C(=O)N(C1CCC(=O)NC1=O)C2=O]

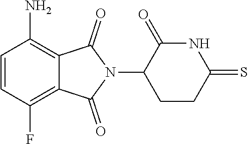 [SMILES: Nc1ccc(F)c2c1C(=O)N(C1CCC(=S)NC1=O)C2=O]

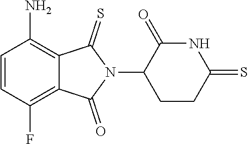 [SMILES: Nc1ccc(F)c2c1C(=S)N(C1CCC(=S)NC1=O)C2=O]

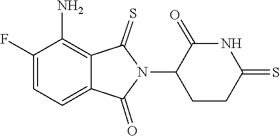 [SMILES: Nc1c(F)ccc2c1C(=S)N(C1CCC(=S)NC1=O)C2=O]

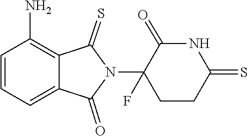 [SMILES: Nc1cccc2c1C(=S)N(C1(F)CCC(=S)NC1=O)C2=O]

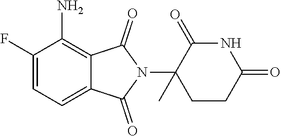 [SMILES: CC1(N2C(=O)c3ccc(F)c(N)c3C2=O)CCC(=O)NC1=O]

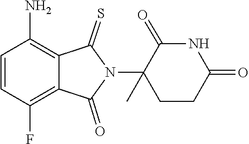 [SMILES: CC1(N2C(=O)c3c(F)ccc(N)c3C2=S)CCC(=O)NC1=O]

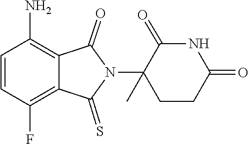 [SMILES: CC1(N2C(=O)c3c(N)ccc(F)c3C2=S)CCC(=O)NC1=O]

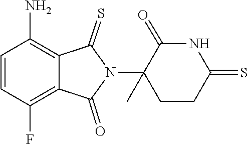 [SMILES: CC1(N2C(=O)c3c(F)ccc(N)c3C2=S)CCC(=S)NC1=O]

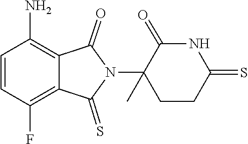 [SMILES: CC1(N2C(=O)c3c(N)ccc(F)c3C2=S)CCC(=S)NC1=O]

Advantageously, some embodiments of the disclosed halophthalimides reduce undesired oxidative metabolism due to the increased strength of the C—X bond compared C—H bonds. For example, a typical C—F bond has a strength of 116 kcal/mol relative to an analogous C—H bond with a strength of 99 kcal/mol (Park et al., Annu Rev Pharmacol Toxicol 2001, 41:443-470). Additionally, the compounds may exhibit greater stability in vivo since redox-active liver enzymes may be less able to disrupt the C—X bonds, thereby impeding drug processing by the body. The increased lipophilicity provided by halogenation also may enhance bioavailability and/or pharmacokinetic properties. With respect to fluorinated analogs, the similar van der Waals radii of F (1.47 Å) and H (1.20 Å) makes the substitution ideal for molecules involved in sterically influenced processes, such as enzyme-ligand binding interactions. Fluorination may increase blood brain barrier permeability, improve resistance to chemical degradation, increase metabolic stability, and/or enhance binding to target macromolecules. Thionation and/or inclusion of a bridged carbocycle at R⁵ also may increase pharmacokinetic properties.

### III. Pharmaceutical Compositions

Embodiments of a pharmaceutical composition include one or more halophthalimides, or a stereoisomer, pharmaceutically acceptable salt, solvate, or a hydrate thereof, and a pharmaceutically acceptable carrier. The disclosed compounds can be further combined with excipients, and optionally sustained-release matrices, such as biodegradable polymers. The composition may comprise a unit dosage form of the composition, and may further comprise instructions for administering the composition to a subject. Such pharmaceutical compositions may be used in methods for inhibiting TNF-α activity. TNF-α synthesis, inflammation, inducible nitric oxide synthase (iNOS), SARS-CoV-2 virus, or any combination thereof, as well as diseases and disorders characterized by abnormal levels of TNF-α activity, TNF-α synthesis, inflammation, and/or inducible nitric oxide synthase (iNOS), as discussed further in section IV below.

The disclosed pharmaceutical compositions can be in the form of tablets, capsules, powders, granules, lozenges, liquid or gel preparations, such as oral, topical, or sterile parenteral solutions or suspensions (e.g., eye or ear drops, throat or nasal sprays, etc.), transdermal patches, and other forms known in the art.

Pharmaceutical compositions can be administered systemically or locally in any manner appropriate to the treatment of a given condition, including orally, parenterally, rectally, nasally, buccally, vaginally, topically, optically, by inhalation spray, or via an implanted reservoir. The term “parenterally” as used herein includes, but is not limited to subcutaneous, intravenous, intramuscular, intrasternal, intrasynovial, intrathecal, intrahepatic, intralesional, and intracranial administration, for example, by injection or infusion. For treatment of the central nervous system, the pharmaceutical compositions may readily penetrate the blood-brain barrier when peripherally or intraventricularly administered.

Pharmaceutically acceptable carriers include, but are not limited to, ion exchangers, alumina, aluminum stearate, lecithin, serum proteins (such as human serum albumin), buffers (such as phosphates), glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Tablets and capsules for oral administration can be in a form suitable for unit dose presentation and can contain conventional pharmaceutically acceptable excipients. Examples of these include binding agents such as syrup, acacia, gelatin, sorbitol, tragacanth, and polyvinylpyrrolidone; fillers such as lactose, sugar, corn starch, calcium phosphate, sorbitol, or glycine; tableting lubricants, such as magnesium stearate, talc, polyethylene glycol, or silica; disintegrants, such as potato starch; and dispersing or wetting agents, such as sodium lauryl sulfate. Oral liquid preparations can be in the form of, for example, aqueous or oily suspensions, solutions, emulsions, syrups or elixirs, or can be presented as a dry product for reconstitution with water or other suitable vehicle before use.

The pharmaceutical compositions can also be administered parenterally in a sterile aqueous or oleaginous medium. The composition can be dissolved or suspended in a non-toxic parenterally-acceptable diluent or solvent, e.g., as a solution in 1,3-butanediol. Commonly used vehicles and solvents include water, physiological saline, Hank's solution, Ringer's solution, and sterile, fixed oils, including synthetic mono- or di-glycerides, etc. For topical application, the drug may be made up into a solution, suspension, cream, lotion, or ointment in a suitable aqueous or non-aqueous vehicle. Additives may also be included, for example, buffers such as sodium metabisulfite or disodium edetate; preservatives such as bactericidal and fungicidal agents, including phenyl mercuric acetate or nitrate, benzalkonium chloride or chlorhexidine, and thickening agents, such as hypromellose.

The compounds can be used in the form of pharmaceutically acceptable salts derived from inorganic or organic acids and bases, including, but not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include, but are not limited to, ammonium salts, alkali metal salts (such as sodium and potassium salts), alkaline earth metal salts (such as calcium and magnesium salts), salts with organic bases (such as dicyclohexylamine salts), N-methyl-D-glucamine, and salts with amino acids (such as arginine, lysine, etc.). Basic nitrogen-containing groups can be quaternized, for example, with such agents as C1-8 alkyl halides (such as methyl, ethyl, propyl, and butyl chlorides, bromides, and iodides), dialkyl sulfates (such as dimethyl, diethyl, dibutyl, and diamyl sulfates), long-chain halides (such as decyl, lauryl, myristyl, and stearyl chlorides, bromides, and iodides), aralkyl halides (such as benzyl and phenethyl bromides), etc. Water or oil-soluble or dispersible products are produced thereby.

### IV. Methods of Use

The compounds disclosed herein, and stereoisomers, pharmaceutically acceptable salts, solvates, or hydrates thereof, may be used for inhibiting TNF-α activity, TNF-α synthesis, inflammation, inducible nitric oxide synthase (iNOS), SARS-CoV-2 virus, or any combination thereof. Advantageously, some embodiments of the disclosed halophthalimides demonstrate greater inhibition of one or more of TNF-α activity, TNF-α synthesis, inflammation, iNOS, and SARS-CoV-2 compared to non-halogenated phthalimides. In some embodiments, thionation also provided greater efficacy compared to corresponding non-thionated analogs. In particular examples, a combination of 7-fluoro substitution and thionation provided increased anti-nitrite activity with minimal cellular toxicity. In some examples, compounds including a bridged carbocycle also exhibit potent anti-nitrite and/or anti-TNF-α activity with minimal cellular toxicity. In certain examples, compounds comprising a bridged carbocycle are effective anti-viral agents. Some compounds also demonstrate heightened stability in human liver microsomes compared to corresponding non-halogenated analogs.

In one embodiment, a cell is contacted with an effective amount of a halophthalimide as disclosed herein, to inhibit TNF-α activity, TNF-α synthesis, inflammation, inducible nitric oxide synthase (iNOS), SARS-CoV-2 virus, or any combination thereof. The cell may be contacted in vitro, in vivo, or ex vivo. In one embodiment, the cell is contacted with a halophthalimide as disclosed in any one of Tables 1-5.

In any of the foregoing embodiments, contacting the cell with an effective amount of the halophthalimide may comprise administering to a subject a therapeutically effective amount of the halophthalimide, or stereoisomer, pharmaceutically acceptable salt, solvate, or hydrate thereof, or a therapeutically effective amount of a pharmaceutical composition comprising the halophthalimide or stereoisomer, pharmaceutically acceptable salt, solvate, or hydrate thereof. Administration may be performed by any suitable route, including orally, parenterally, rectally, nasally, buccally, vaginally, topically, optically, by inhalation spray, or via an implanted reservoir.

In one embodiment, a subject is administered a therapeutically effective amount of halophthalimide according to general formula I, or stereoisomer, a pharmaceutically acceptable salt, solvate, or hydrate thereof, or a pharmaceutical composition comprising the compound. In another embodiment, a subject is administered a therapeutically effective amount of a halophthalimide as disclosed in any one of Tables 1-5, or a stereoisomer, a pharmaceutically acceptable salt, solvate, or hydrate thereof, or a pharmaceutical composition comprising the compound.

In some implementations, the subject has an inflammatory disorder, an autoimmune disorder, a neurodegenerative disease, a viral infection, or any combination thereof. In some implementations, the subject has a viral infection, particularly a coronavirus infection, such as a SARS-CoV-2 infection. Exemplary inflammatory and/or autoimmune disorders that may be ameliorated with embodiments of the disclosed halophthalimides include, but are not limited to, neuroinflammation, rheumatoid arthritis, immune arthritis, degenerative arthritis, celiac disease, glomerulonephritis, lupus nephritis, prostatitis, inflammatory bowel disease (e.g., Crohn's disease), pelvic inflammatory disease, graft-versus-host disease, interstitial cystitis, autoimmune thyroiditis, Graves' disease; autoimmune pancreatitis, Sjogren's syndrome, myocarditis, autoimmune hepatitis, primary biliary cirrhosis, autoimmune angioedema, bullous pemphigoid, discoid lupus erythematosus, erythema nodosum leprosum, sarcoidosis, pemphigus vulgaris psoriasis, POEMS syndrome, polymyositis, human immune deficiency virus/acquired immune deficiency syndrome, vasculitis, and sarcopenia. In some embodiments, the subject has a traumatic brain injury (TBI) and/or neuroinflammation following a TBI. In some embodiments, the neurodegenerative disease is Alzheimer's disease (AD), Parkinson's disease (PD), multiple sclerosis (MS), amyotrophic lateral sclerosis (ALS), or Huntington's disease (HD). Subject to neurotoxicity considerations (e.g., whether the halophthalimide is well tolerated by nervous tissue), certain embodiments of the halophthalimides disclosed herein may be used to reduce neuroinflammation as a treatment strategy for neurodegenerative disorders. Advantageously, a halophthalimide used to reduce neuroinflammation may be non-neurotoxic at a therapeutically effective dose. Examples of neurodegenerative and/or neuroinflammatory disorders that may be ameliorated with embodiments of the disclosed halophthalimides include, but are not limited to, neurodegeneration resulting from head trauma (e.g., traumatic brain injury), spinal cord injuries, stroke, Alzheimer's disease, Parkinson's disease, ALS (amyotrophic lateral sclerosis), HIV (human immunodeficiency virus) dementia, Huntington's disease, multiple sclerosis, cerebral amyloid angiopathy, tauopathies, peripheral neuropathies, macular degeneration, hearing loss, cochlear injury, epilepsy, a non-epileptic seizure disorder (e.g., due to head injury, dementia, prenatal brain injury, meningitis, lupus, encephalitis, among others), and major depressive disorder (also known as clinical depression, unipolar depression). Embodiments of the disclosed halophthalimides may be used to reduce chronic systemic and CNS inflammation and/or as immunomodulatory agents. Embodiments of the disclosed halophthalimides are small molecular weight lipophilic compounds with physicochemical properties that may allow them to pass through the blood-brain barrier.

TNF-α serves as a regulator in acute stages of neuroinflammation, triggering signaling cascades of pro-inflammatory cytokines. Increased TNF-α is associated with several neurodegenerative disorders, including TBI, AD, PD, MS, ALS, and HD, among others. Advantageously, some embodiments of the disclosed compounds inhibit TNF-α and/or ameliorate inflammation without binding to cereblon (the primary target of thalidomide teratogenicity). Reduced inflammation may be evidenced by reduced levels of pro-inflammatory cytokines and/or chemokines (e.g., TNF-α, IL-1, IL-2, IL-5, IL-6, IL-12, IL-17, IL-18, IFN-γ, KC/GRO (CXCL1), CXCL2, CXCL8, CCL2, CCL3, CCL5) in plasma and/or brain tissue.

TBI is a leading cause of death and disability in children and adults. TBI has been identified as a major risk factor for several neurodegenerative disorders, including PD and AD. Neuroinflammation is considered the cause of later secondary cell death following TBI, and has the potential to chronically aggravate the first impact. Within minutes to hours after TIB, mRNA and protein expression of TNF-α is elevated. Advantageously, some embodiments of the disclosed compounds mitigate lipopolysaccharide-induced inflammation and TNF-α levels, and decrease neuroinflammation induced by controlled cortical impact. In some embodiments, the disclosed compounds decrease lesion size/volume following TBI, compared to lesion size/volume in the absence of treatment with the disclosed compounds. In some implementations, the disclosed compounds further mitigate microglial cell activation, neuronal loss, and/or behavioral deficits when administered after TBI. For example, following administration of an effective amount of a disclosed halophthalimide, a subject may demonstrate reduced impairment in fine motor coordination and/or balance, following TBI, compared to a subject that has not been treated with a disclosed halophthalimide. The compounds further may be useful for treating longer-term neurodegenerative disorders, such as those disorders mediated by neuroinflammation and/or induced by TBI (e.g., AD, PD, MS, and/or ALS).

In one embodiment, the subject has aberrantly high TNF-α activity and/or nitrite activity, and the subject is administered a therapeutically effective amount of a compound or a pharmaceutically acceptable salt, solvate, or hydrate thereof, or a pharmaceutical composition comprising the compound or pharmaceutically acceptable salt, solvate, or hydrate thereof, wherein the compound is

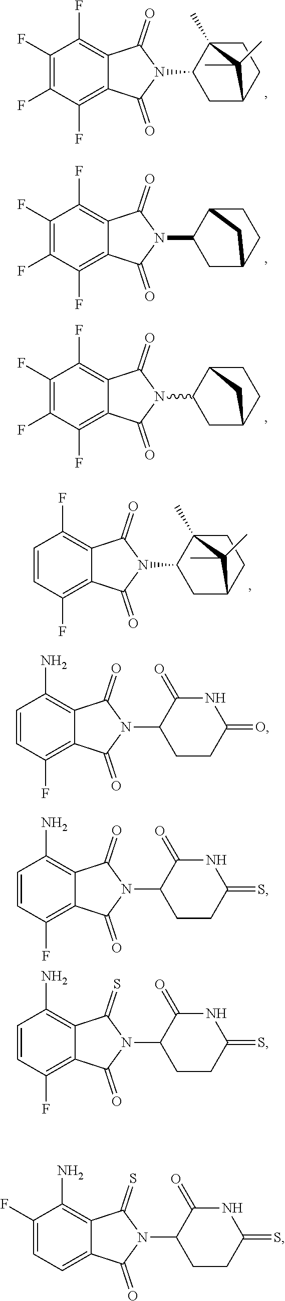

or any combination thereof. In an independent embodiment, the subject has a SARS-CoV-2 infection, and the subject is administered a therapeutically effective amount of a compound or a pharmaceutically acceptable salt, solvate, or hydrate thereof, or a pharmaceutical composition comprising the compound or pharmaceutically acceptable salt, solvate, or hydrate thereof, wherein the compound is

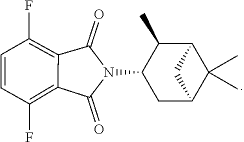 [SMILES: C[C@@H]1[C@@H](N2C(=O)c3c(F)ccc(F)c3C2=O)C[C@H]2C[C@@H]1C2(C)C]

In another independent embodiment, the subject has a TBI and/or TBI-induced neuroinflammation, and the subject is administered a therapeutically effective amount of a compound or a pharmaceutically acceptable salt, solvate, or hydrate thereof, or a pharmaceutical composition comprising the compound or pharmaceutically acceptable salt, solvate, or hydrate thereof, wherein the compound is

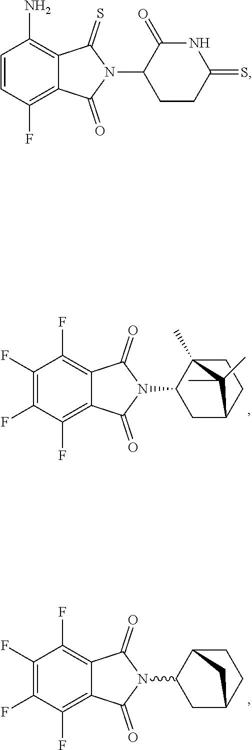

or any combination thereof.

### V. EXAMPLES

### Example 1

### Synthesis of Monoterpenoid Halophthalimides

Compounds comprising fluorophthalimide moieties and a variety of bridged-ring moieties were synthesized. In general, they were prepared by condensation of a fluorophthalic anhydride and an appropriate amine containing bridged ring. The bridged rings included the following monoterpenoid moieties-N-isopinocampheyl, N-bornyl, N-norbornyl, and N-myrtanyl groups. Structures 1-7 below are exemplary monoterpenoids possessing biological activity. Monoterpenoids 1-7 inhibit toxin accumulation, discharge existing toxins, and/or possess anti-inflammatory, anti-oxidation, antiviral, and/or other pharmacological activities.

Compounds 11-22 were synthesized from precursors 1-8.

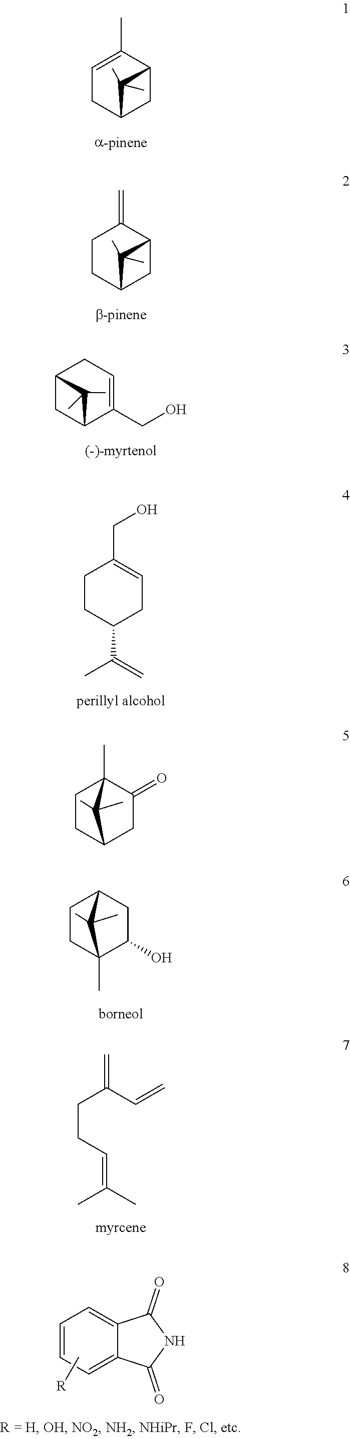

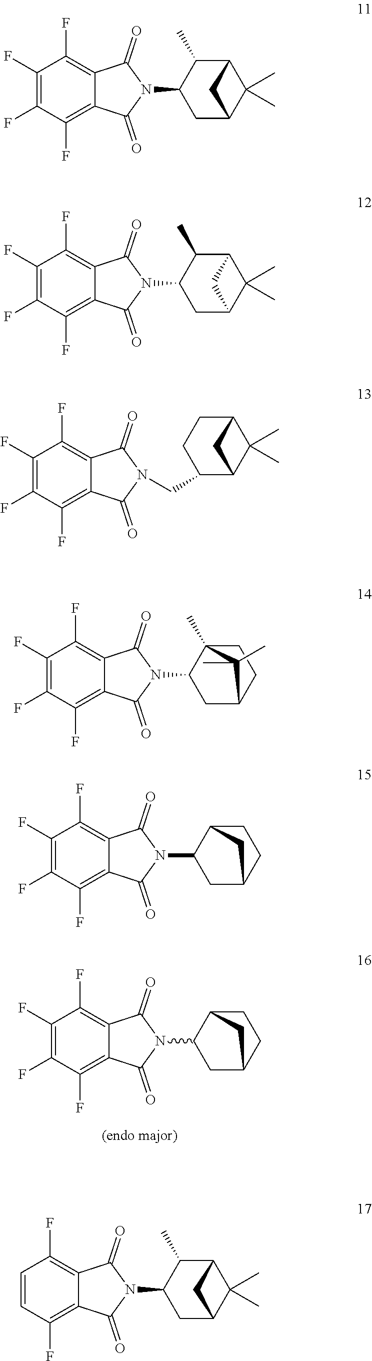

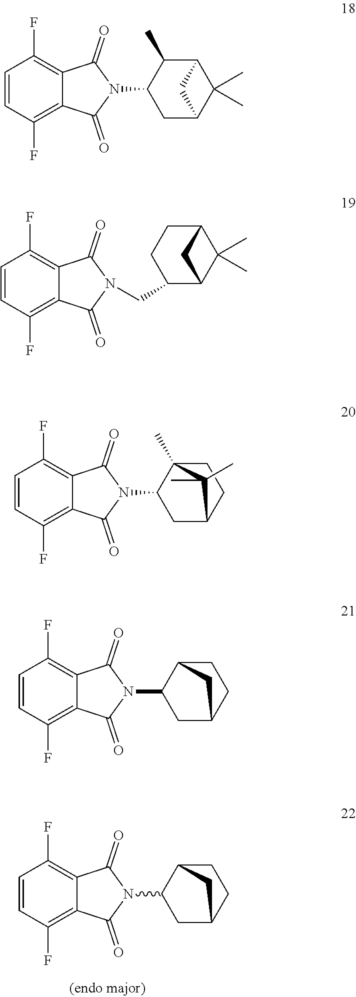

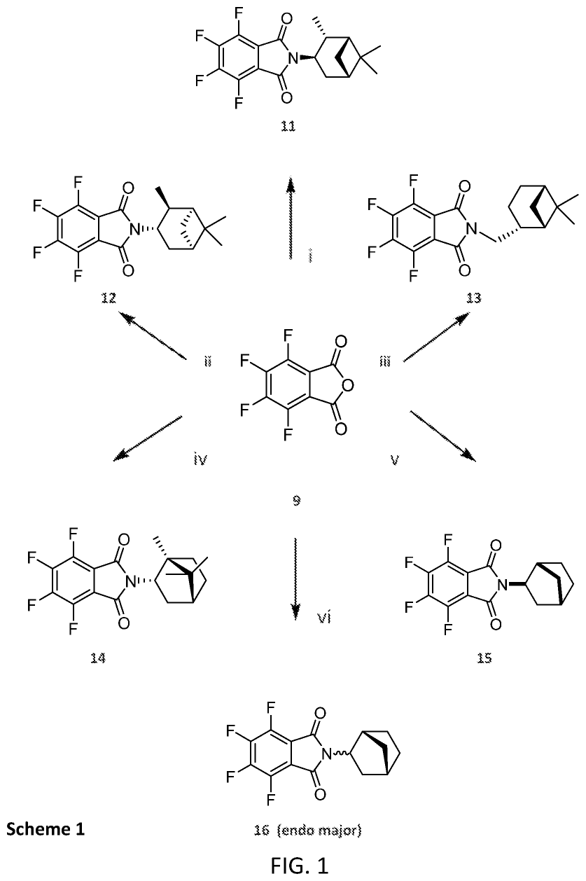

Substituted tetrafluorophthalimides 11-16 were prepared as shown in Scheme 1 (FIG. 1). Reagents and conditions: (i) (1R,2R,3R,5S)-(−)-isopino-campheylamine (for 11), acetic acid; (ii) (1S,2S,3S,5R)-(+)-isopino-campheylamine (for 12), acetic acid; (iii) (−)-cis-myrtanylamine (for 13), acetic acid; (iv) (R)-(+)-bornylamine (for 14), acetic acid; (v) exo-2-aminonorbornane (for 15), acetic acid; (vi) 2-aminonorbornane hydrochloride (for 16), acetic acid. Most of them had acceptable synthetic yield. The compound 13 was generated in 52.3% of yield as result of smaller stereo hindrance of myrtanyl group. The yield of compound 14 was 12.1%, and one of the reasons might be because the condensation underwent a greater stereo hindrance from bornyl group.

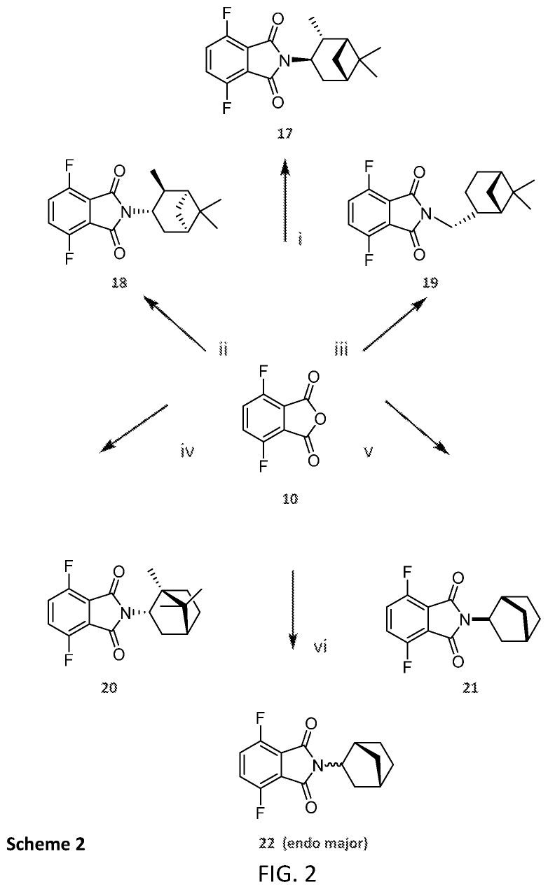

The synthesis of new difluorophthalimides 17-22 is shown in Scheme 2 (FIG. 2). Reagents and conditions: (i) (1R,2R,3R,5S)-(−)-isopino-campheylamine (for 17), acetic acid; (ii) (1S,2S,3S,5R)-(+)-isopino-campheylamine (for 18), acetic acid; (iii) (−)-cis-myrtanylamine (for 19), acetic acid; (iv) (R)-(+)-bornylamine (for 20), acetic acid; (v) exo-2-aminonorbornane (for 21), acetic acid; (vi) 2-aminonorbornane hydrochloride (for 22), acetic acid. Since the condensation reaction of tetra- or di-fluorophthalic anhydride with bridged-ring amine occurred more easily in a decarbonylation, it also affected synthetic yield of the target compounds. Besides, due to well-known 2-norbornyl solvolyses, 15 and 21 were separately their own exo-racemates. 16 and 22 were primarily their own endo-diastereomer, respectively.

For compounds 11-16, a mixture of 353-1330 mg (1.60-6.04 mmol) of tetrafluorophthalic anhydride 9 and equal molar amount of corresponding bridged-ring amines (if utilized as the hydrochloride salt, an equal molar amount of triethylamine was added) in 8-29 mL of acetic acid was stirred for 3.0-18.0 h under a nitrogen atmosphere at 100-120° C. (oil bath). After removing solvent, the residues were purified by silica gel chromatography plate (MeOH/CH₂Cl₂ or CH₂Cl₂) and/or recrystallized from acetone.

For compounds 17-22, a mixture of 70-556 mg (0.38-3.02 mmol) of difluorophthalic anhydride 10 and equal molar amount of corresponding bridged-ring amines (if utilized as the hydrochloride salt, equal molar amount of triethylamine was added) in 3-15 mL of acetic acid was stirred for 3.0-92.0 h under a nitrogen atmosphere at 100° C. (oil bath). After removing solvent, the residues were purified by silica gel chromatography plate (MeOH/CH₂Cl₂) and/or recrystallized from acetone.

Melting points (uncorrected) were measured with a Fisher-Johns apparatus. ¹H-NMR, ¹⁹F-NMR and ¹³C-NMR spectra were gotten on a Varian Mercury Plus 400 spectrometer at 400 MHz, 377 MHz and 101 MHz at room temperature. GC/MS (m/z) spectra were measured on an Agilent Technologies (Santa Clara, CA) 7890B GC equipped with an HP-5 MS column and a 5977B mass-selective ion detector in the electron-impact mode. LC/MS spectra were recorded on an Agilent 6125-LC-MS Single Quad system. HRMS (ESI+) data were obtained on an Orbitrap Velos mass spectrometer (Thermo Fisher) with a HESI source. Elemental analyses were performed by Atlantic Microlab, Inc. All isopinocampheylamine, myrtanylamine, bornylamine, exo-2-aminonorbornane and 2-aminonorbornane hydrochloride were purchased from Sigma-Aldrich Chemical Company and used as received.

4,5,6,7-Tetrafluoro-2-((1R,2R,3R,5S)-2,6,6-trimethylbicyclo[3.1.1]heptan-3-yl) isoindoline-1,3-dione (11): White crystals (21.7%, chromatography from CH₃OH/CH₂Cl₂); mp 150.0-151.5° C.; ¹H-NMR (CDCl₃) δ 4.65 (q, J=8.6 Hz, 1H), 2.51 (t, J=7.4 Hz, 1H), 2.38 (d, J=8.2 Hz, 1H), 2.29 (t, J=12.5 Hz, 1H), 2.23 (dd, J=12.1, 13.3 Hz, 1H), 2.06 (s, 1H), 1.91 (t, J=5.5 Hz, 1H), 1.80 (d, J=9.8 Hz, 1H), 1.28 (s, 3H), 1.13 (s, 3H) and 1.02 (d, J=7.0 Hz, 3H) ppm; ¹³C-NMR (CDCl₃) δ 162.8, 144.9 (d, J_{CF}=269.3 Hz), 143.3 (d, J_{CF}=260.9 Hz), 113.6, 50.2, 48.2, 42.0, 40.3, 39.1, 32.8, 31.6, 28.2, 23.8 and 20.3 ppm; ¹⁹F-NMR (CDCl₃) δ −137.0 (d, J=10.9 Hz, 2F) and −143.3 (d, J=10.9 Hz, 2F) ppm; GC-MS (CI/CH₄), m/z 356 (MH⁺); HRMS, ESI (+), calcd for C₁₈H₁₈F₄NO₂: 356.1268, found: 356.1265.
4,5,6,7-Tetrafluoro-2-((1S,2S,3S,5R)-2,6,6-trimethylbicyclo[3.1.1]heptan-3-yl) isoindoline-1,3-d ione (12): White crystals (16.1%, chromatography from CH₃OH/CH₂Cl₂); mp 150.2-151.6° C.; ¹H-NMR (CDCl₃) δ 4.65 (q, J=8.6 Hz, 1H), 2.51 (t, J=7.4 Hz, 1H), 2.38 (d, J=8.2 Hz, 1H), 2.29 (t, J=12.5 Hz, 1H), 2.23 (dd, J=12.5, 12.9 Hz, 1H), 2.06 (s, 1H), 1.90 (s, 1H), 1.80 (d, J=9.8 Hz, 1H), 1.28 (s, 3H), 1.13 (s, 3H) and 1.02 (d, J=7.0 Hz, 3H) ppm; ¹³C-NMR (CDCl₃) δ 162.8, 144.9 (d, J_{CF}=269.3 Hz), 143.3 (d, J_{CF}=260.9 Hz), 113.6, 50.2, 48.2, 42.0, 40.3, 39.1, 32.8, 31.6, 28.2, 23.8 and 20.3 ppm; ¹⁹F-NMR (CDCl₃) δ −137.0 (d, J=10.9 Hz, 2F) and −143.3 (d, J=10.9 Hz, 2F) ppm; GC-MS (CI/CH₄), m/z 356 (MH⁺); Anal. Calcd for C₁₈H₁₇F₄NO₂: C, 60.84; H, 4.82 and N, 3.94. Found: C, 60.78; H, 4.86 and N, 3.96.
2-(((1S,2R,5S)-6,6-dimethylbicyclo[3.1.1]heptan-2-yl)methyl)-4,5,6,7-tetrafluoroisoindoline-1,3-dione (13): White needle crystals (52.3%, chromatography from CH₃OH/CH₂Cl₂); mp 160.0-161.0° C.; ¹H-NMR (CDCl₃) δ 3.70-3.64 (m, 2H), 2.48 (s, 1H), 2.33 (s, 1H), 1.98-1.80 (m, 5H), 1.54 (s, 1H), 1.20 (s, 3H), 1.17 (s, 3H) and 0.86 (d, J=9.4 Hz, 1H) ppm; ¹³C-NMR (CDCl₃) δ 162.7, 144.9 (d, J_{CF}=256.4 Hz), 143.2 (d, J_{CF}=265.5 Hz), 113.7 (d, J_{CF}=7.6 Hz), 44.0, 43.2, 41.1, 39.6, 38.7, 32.9, 27.7, 25.8, 22.8 and 19.1 ppm; ¹⁹F-NMR (CDCl₃) δ −135.8 (t, J=10.9 Hz, 2F) and −142.6 (d, J=10.9 Hz, 2F) ppm; GC-MS (CI/CH₄), m/z 356 (MH⁺); Anal. Calcd for C₁₈H₁₇F₄NO₂: C, 60.84; H, 4.82; F, 21.39 and N, 3.94. Found: C, 61.05; H, 4.87; F, 21.20 and N, 3.98.

X-ray crystallography experimental—All reflection intensities were measured at 110(2) K using a SuperNova diffractometer (equipped with Atlas detector) with Cu Kα radiation (λ=1.54178 Å) under the program CrysAlisPro (Version CrysAlisPro 1.171.39.29c, Rigaku OD, 2017). The same program was used to refine the cell dimensions and for data reduction. The structure was solved with the program SHELXS-2018/3 (Sheldrick, 2018) and was refined on F² with SHELXL-2018/3 (Sheldrick, 2018). Analytical numeric absorption correction using a multifaceted crystal model was applied using CrysAlisPro. The temperature of the data collection was controlled using the system Cryojet (manufactured by Oxford Instruments). The H atoms were placed at calculated positions (unless otherwise specified) using the instructions AFIX 13, AFIX 23 or AFIX 137 with isotropic displacement parameters having values 1.2 or 1.5 U_{eq} of the attached C atoms. The structure is ordered.

The absolute configuration has been established by anomalous-dispersion effects in diffraction measurements on the crystal, and the Flack and Hooft parameters refine to 0.02(2) and 0.016 (19), respectively. 791_ALERT_4_G Model has Chirality at C10 (Sohnke SpGr) R Verify; 791_ALERT_4_G Model has Chirality at C13 (Sohnke SpGr) S Verify; 791_ALERT_4_G Model has Chirality at C15 (Sohnke SpGr) S Verify.

4,5,6,7-Tetrafluoro-2-((1R,2S,4R)-1,7,7-trimethylbicyclo[2.2.1]heptan-2-yl) isoindoline-1,3-dio ne (14): A mixture of 1.33 g (6.04 mmol) of tetrafluorophthalic anhydride and 0.925 g (6.04 mmol) of (R)-(+)-bornylamine in 29 mL of acetic acid was stirred for 18.0 h under a nitrogen atmosphere at 120° C. (oil bath). After removing the solvent, the residues were first purified by silica gel chromatography plate (CH₂Cl₂), and then recrystallized twice with acetone to yield 0.26 g of 14 as white crystals. White crystals (12.1%, chromatography from CH₂Cl₂ and then recrystallization from acetone): mp 131.5-132.5° C.; ¹H-NMR (CDCl₃) δ 4.51 (qq, J=2.7, 2.7 Hz, 1H), 2.41 (dd, J=5.5, 5.5 Hz, 1H), 1.97 (ttt, J=4.7, 3.7, 4.6 Hz, 1H), 1.88-1.72 (m, 3H), 1.53-1.46 (m, 1H), 1.37 (qqq, J=2.5, 2.4, 2.6 Hz, 1H), 1.02 (s, 3H), 0.92 (s, 3H) and 0.80 (s, 3H) ppm; ¹³C-NMR (CDCl₃) δ 164.0, 144.9 (d, J_{CF}=263.6 Hz), 143.2 (d, J_{CF}=266.7 Hz), 113.6 (d, J_{CF}=6.9 Hz), 59.1, 51.9, 47.8, 45.5, 29.9, 27.0, 26.8, 19.7, 18.7 and 14.1 ppm; ¹⁹F-NMR (CDCl₃) δ −137.0 (d, J=10.9 Hz, 2F) and −142.9 (d, J=12.3 Hz, 2F) ppm; LC-MS, ESI (+), m/z 356 (MH⁺); Anal. Calcd for C₁₈H₁₇F₄NO₂: C, 60.84; H, 4.82; F, 21.39 and N, 3.94. Found: C, 60.79; H, 4.83; F, 21.47 and N, 3.93.

X-ray crystallography experimental-All reflection intensities were measured at 110(2) K using a SuperNova diffractometer (equipped with Atlas detector) with Cu Kα radiation (λ=0.71073 Å) under the program CrysAlisPro (Version CrysAlisPro 1.171.39.29c, Rigaku OD, 2017). The same program was used to refine the cell dimensions and for data reduction. The structure was solved with the program SHELXS-2018/3 (Sheldrick, 2018) and was refined on F² with SHELXL-2018/3 (Sheldrick, 2018). Analytical numeric absorption correction using a multifaceted crystal model was applied using CrysAlisPro. The temperature of the data collection was controlled using the system Cryojet (manufactured by Oxford Instruments). The H atoms were placed at calculated positions using the instructions AFIX 13, AFIX 23 or AFIX 137 with isotropic displacement parameters having values 1.2 or 1.5 U_{eq} of the attached C atoms. The structure is ordered.

The absolute structure configuration has been established by anomalous dispersion effects in diffraction measurements on the crystal. The Flack and Hooft parameters refine to 0.011 (19) and 0.007 (16), respectively. 791 ALERT_4_G Model has Chirality at C9 (Sohnke SpGr) S Verify; 791 ALERT_4_G Model has Chirality at C11 (Sohnke SpGr) R Verify; 791 ALERT 4_G Model has Chirality at C14 (Sohnke SpGr) R Verify.

2-Exo-(bicyclo[2.2.1]heptan-2-yl)-4,5,6,7-tetrafluoroisoindoline-1,3-dione (15): White solid (15.7%, chromatography from CH₃OH/CH₂Cl₂); mp 209.0-210.0° C.; ¹H-NMR (CDCl₃) δ 4.09 (t, J=5.9 Hz, 1H), 2.42 (d, J=12.9 Hz, 2H), 2.16 (t, J=11.5 Hz, 2H), 1.73 (t, J=10.6 Hz, 1H), 1.62-1.52 (m, 2H), 1.33-1.19 (m, 3H) ppm; ¹³C-NMR (CDCl₃) δ 163.0, 144.8 (d, J_{CF}=268.6 Hz), 143.2 (d, J_{CF}=261.7 Hz), 113.6, 56.4, 42.0, 37.1, 36.3, 36.0, 28.8 and 27.9 ppm; ¹⁹F-NMR (CDCl₃) δ −137.0 (t, J=10.2 Hz, 2F) and −143.0 (1, J=10.2 Hz, 2F) ppm; GC-MS (CI/CH₄), m/z 314 (MH⁺); Anal. Calcd for C₁₅H₁₁F₄NO₂: C, 57.51; H, 3.54 and N, 4.47. Found: C, 57.55; H, 3.53 and N, 4.56. The X-ray crystallography analysis showed this compound was the racemates.

X-ray crystallography experimental-All reflection intensities were measured at 110(2) K using a SuperNova diffractometer (equipped with Atlas detector) with Cu Kα radiation (λ=1.54178 Å) under the program CrysAlisPro (Version CrysAlisPro 1.171.39.29c, Rigaku OD, 2017).

The same program was used to refine the cell dimensions and for data reduction. The structure was solved with the program SHELXS-2018/3 (Sheldrick, 2018) and was refined on F² with SHELXL-2018/3 (Sheldrick, 2018). Analytical numeric absorption correction using a multifaceted crystal model was applied using CrysAlisPro. The temperature of the data collection was controlled using the system Cryojet (manufactured by Oxford Instruments). The H atoms were placed at calculated positions using the instructions AFIX 13 or AFIX 23 with isotropic displacement parameters having values 1.2 U_{eq} of the attached C atoms. The structure is partly disordered.

The fragment C9→C15 is disordered over three orientations, and the occupancy factors of the three components of the disorder refine to 0.508(3), 0.289(2) and 0.203(3).

2-Endo-(bicyclo[2.2.1]heptan-2-yl)-4,5,6,7-tetrafluoroisoindoline-1,3-dione (16): A mixture of 1.33 g (6.04 mmol) of tetrafluorophthalic anhydride, 0.892 g (6.04 mmol) of 2-aminonorbornane hydrochloride and 0.611 g (6.04 mmol) of triethylamine in 29 mL of acetic acid was stirred for 36.5 h under a nitrogen atmosphere at 100° C. (oil bath). After removing solvent, the residues were first purified by silica gel chromatography plate (CH₂Cl₂), and then recrystallized with acetone to yield 0.23 g of 16 as white crystals. White flake crystals (12.2%, chromatography from CH₃OH/CH₂Cl₂ and then recrystallization from acetone); mp 173.0-174.0° C.; ¹H-NMR (CDCl₃) δ 4.43-4.38 (m, 1H), 4.12-4.07* (m, 0.1H), 2.54 (s, 1H), 2.46-2.40 (m, 2H), 2.23-2.13* (m, 0.24H), 1.82-1.70 (m, 2H), 1.63-1.35 (m, 5H) and 1.35-1.18* (0.29 H) ppm; ¹³C-NMR (CDCl₃) δ 163.7, 144.8 (d, J_{CF}=269.3 Hz), 143.2 (d, J_{CF}=263.2 Hz), 113.6, 56.4*, 55.7, 42.3, 42.0*, 37.9, 37.3, 37.1*, 36.3*, 36.0*, 28.8, 27.9 and 23.3 ppm; ¹⁹F-NMR (CDCl₃) δ −136.9 (m, 2F) and −142.9 (m, 2F) ppm; LC-MS, ESI (+), m/z 314 (MH⁺); Anal. Calcd for C₁₅H₁₁F₄NO₂: C. 57.51; H, 3.54, F, 24.26 and N, 4.47. Found: C, 57.59; H, 3.54; F, 24.06 and N, 4.47. [*The data came from the 2-exo-diastereomer of 16. 2-Chiral starting material of 16 came from 2-aminonorbornane hydrochloride produced by Sigma-Aldrich Company and it also showed out similar minor 2-exo-aminonorbornane hydrochloride diastereomer in its ¹H-NMR (in solvent DMSO-d₆)]. The X-ray crystallography analysis showed this compound was not enantiomerically pure. The NMR and X-ray crystallographic data exhibited compound 16 was primarily 2-endo diastereomer.

X-ray crystallography experimental-All reflection intensities were measured at 200(2) K using a SuperNova diffractometer (equipped with Atlas detector) with Cu Kα radiation (λ=1.54178 Å) under the program CrysAlisPro (Version CrysAlisPro 1.171.39.29c, Rigaku OD, 2017). The same program was used to refine the cell dimensions and for data reduction. The structure was solved with the program SHELXS-2018/3 (Sheldrick, 2018) and was refined on F² with SHELXL-2018/3 (Sheldrick, 2018). Empirical absorption correction using spherical harmonics was applied using CrysAlisPro. The temperature of the data collection was controlled using the system Cryojet (manufactured by Oxford Instruments). The H atoms were placed at calculated positions using the instructions AFIX 13 or AFIX 23 with isotropic displacement parameters having values 1.2 U_{eq} of the attached C atoms. The structure is disordered as the whole molecule is disordered over two orientations. The occupancy factor of the major component of the disorder refines to 0.7799(18).

The crystal that was mounted on the diffractometer was twinned with two components, and the twin relationship corresponds to a twofold axis along the c* reciprocal axis. The BASF scale factor refines to 0.323 (2).

4,7-Difluoro-2-((1R,2R,3R,5S)-2,6,6-trimethylbicyclo[3.1.1]heptan-3-yl) isoindoline-1,3-dione (17): White crystals (19.0%, chromatography from CH₃OH/CH₂Cl₂); mp 89.5-90.5° C.; ¹H-NMR (CDCl₃) δ 7.36 (s, 2H), 4.66 (q, J=8.6 Hz, 1H), 2.54 (t, J=7.0 Hz, 1H), 2.37 (d, J=7.0 Hz, 1H), 2.32-2.21 (m, 2H), 2.05 (s, 1H), 1.90-1.84 (m, 2H), 1.28 (s, 3H), 1.14 (s, 3H) and 1.02 (d, J=7.1 Hz, 3H) ppm; ¹³C-NMR (CDCl₃) δ 164.3, 153.6 (d, J_{CF}=264.0 Hz), 124.6 (t, J_{CF}=15.3 Hz), 118.7 (t, J_{CF}=7.6 Hz), 49.6, 48.3, 42.1, 40.2, 39.1, 32.8, 31.6, 28.2, 23.8 and 20.3 ppm; ¹⁹F-NMR (CDCl₃) δ −118.5 (s, 2F) ppm; GC-MS (CI/CH₄), m/z 320 (MH⁺); Anal. Calcd for C₁₈H₁₉F₂NO₂: C, 67.70; H, 6.00 and N, 4.39. Found: C, 67.76; H, 5.92 and N, 4.34.
4,7-Difluoro-2-((1S,2S,3S,5R)-2,6,6-trimethylbicyclo[3.1.1]heptan-3-yl) isoindoline-1,3-dione (18): White crystals (21.3%, chromatography from CH₃OH/CH₂Cl₂); mp 89.0-90.0° C.; ¹H-NMR (CDCl₃) δ 7.36 (d, J=5.1 Hz, 2H), 4.66 (q. J=8.6 Hz, 1H), 2.54 (t, J=7.2 Hz, 1H), 2.37 (d, J=7.0 Hz, 1H), 2.33-2.20 (m, 2H), 2.05 (s, 1H), 1.90-1.84 (m, 2H), 1.28 (s, 3H), 1.14 (s, 3H) and 1.02 (d, J=7.4 Hz, 3H) ppm; ¹³C-NMR (CDCl₃) δ 164.6, 153.6 (d, J_{CF}=264.0 Hz), 124.9 (t, J_{CF}=15.3 Hz), 119.1, 50.0, 48.7, 42.5, 40.6, 39.4, 33.2, 32.0, 28.6, 24.2 and 20.7 ppm; ¹⁹F-NMR (CDCl₃) δ −118.3 (s, 2F) ppm; GC-MS (CI/CH₄), m/z 320 (MH⁺); Anal. Calcd for C₁₈H₁₉F₂NO₂: C, 67.70; H, 6.00 and N, 4.39. Found: C, 67.68; H, 6.08 and N, 4.40.

X-ray crystallography experimental-All reflection intensities were measured at 110(2) K using a SuperNova diffractometer (equipped with Atlas detector) with Cu Kα radiation (λ=1.54178 Å) under the program CrysAlisPro (Version CrysAlisPro 1.171.39.29c, Rigaku OD, 2017). The same program was used to refine the cell dimensions and for data reduction. The structure was solved with the program SHELXS-2018/3 (Sheldrick, 2018) and was refined on F² with SHELXL-2018/3 (Sheldrick, 2018). Analytical numeric absorption correction using a multifaceted crystal model was applied using CrysAlisPro. The temperature of the data collection was controlled using the system Cryojet (manufactured by Oxford Instruments). The H atoms were placed at calculated positions (unless otherwise specified) using the instructions AFIX 13, AFIX 23, AFIX 43 or AFIX 137 with isotropic displacement parameters having values 1.2 U_{eq} of the attached C atoms. The structure is ordered.

The absolute configuration has been established by anomalous dispersion effects in diffraction measurements on the crystal, and the Flack and Hooft parameters refine to 0.03(3) and 0.05(2), respectively. 791_ALERT_4_G Model has Chirality at C9A (Sohnke SpGr) S Verify; 791_ALERT_4_G Model has Chirality at C9B (Sohnke SpGr) S Verify; 791_ALERT_4_G Model has Chirality at C11A (Sohnke SpGr) R Verify; 791_ALERT_4_G Model has Chirality at C11B (Sohnke SpGr) R Verify; 791_ALERT_4_G Model has Chirality at C13A (Sohnke SpGr) S Verify; 791_ALERT_4_G Model has Chirality at C13B (Sohnke SpGr) S Verify; 791_ALERT_4_G Model has Chirality at C14A (Sohnke SpGr) S Verify; 791_ALERT_4_G Model has Chirality at C14B (Sohnke SpGr) S Verify.

2-(((1S,2R,5S)-6,6-dimethylbicyclo[3.1.1]heptan-2-yl)methyl)-4,7-difluoroisoindoline-1,3-dione (19): White crystals (97.9%, chromatography from CH₃OH/CH₂Cl₂); mp 130.0-131.5° C.; ¹H-NMR (CDCl₃) δ 7.36 (s, 2H), 3.74-3.61 (m, 2H), 2.51 (t, J=8.0 Hz, 1H), 2.33 (d, J=7.8 Hz, 1H), 2.01-1.83 (m, 5H), 1.59-1.55 (m, 1H), 1.20 (s, 3H), 1.18 ((s, 3H) and 0.86 (d, J=9.8 Hz, 1H) ppm; ¹³C-NMR (CDCl₃) δ 164.2, 153.6 (d, J_{CF}=264.0 Hz), 124.6 (d, J_{CF}=17.6 Hz), 118.7, 43.6, 43.3, 41.2, 39.7, 38.7, 33.0, 27.8, 25.9, 22.9 and 19.1 ppm; ¹⁹F-NMR (CDCl₃) δ −118.4 (t, J=5.5 Hz, 2F) ppm; GC-MS (CI/CH₄), m/z 320 (MH⁺); Anal. Calcd for C₁₈H₁₉F₂NO₂: C. 67.70; H, 6.00 and N 4.39. Found: C, 67.89; H, 6.03 and N, 4.22.
4,7-Difluoro-2-((1R,2S,4R)-1,7,7-trimethylbicyclo[2.2.1]heptan-2-yl) isoindoline-1,3-dione (20): White crystals (42.7%, chromatography from MeOH/CH₂Cl₂ and then recrystallization from acetone); mp 137.0-138.0° C.; ¹H-NMR (CDCl₃) δ 7.36 (d, J=5.1 Hz, 2H), 4.54 (d, J=9.8 Hz, 1H), 2.46 (t, J=6.3 Hz, 1H), 1.98-1.75 (m, 4H), 1.59-1.52 (m, 1H), 1.36 (t, J=12.5 Hz, 1H), 1.03 (s, 3H), 0.92 (s, 3H) and 0.82 (s, 3H) ppm; ¹³C-NMR (CDCl₃) δ 165.5, 153.5 (d, J_{CF}=259.4 Hz), 124.4 (d, J_{CF}=17.2 Hz), 118.6, 58.5, 51.8, 47.7, 45.6, 30.0, 27.0, 26.8, 19.7, 18.8 and 14.1 ppm; ¹⁹F-NMR (CDCl₃) δ −119.2 (s, 2F) ppm; GC-MS (CI/CH₄), m/z 320 (MH⁺); Anal. Calcd for C₁₈H₁₉F₂NO₂: C, 67.70; H, 6.00; F, 11.90 and N, 4.39. Found: C, 67.74; H, 6.11; F, 11.76 and N, 4.30.
2-Exo-(bicyclo[2.2.1]heptan-2-yl)-4,7-difluoroisoindoline-1,3-dione (21): White crystals (10.6%, chromatography from CH₃OH/CH₂Cl₂); mp 86.0-87.0° C.; ¹H-NMR (CDCl₃) δ 7.33 (d, J=3.9 Hz, 2H). 4.11 (s, 1H), 2.42 (d, J=10.6 Hz, 2H), 2.21 (d, J=10.6 Hz, 2H), 1.72 (t. J=11.0 Hz, 1H), 1.56 (s, 2H), 1.34 (t, J=8.6 Hz, 1H), 1.27-1.18 (m, 2H) ppm; ¹³C-NMR (CDCl₃) δ 164.4, 153.5 (d, J_{CF}=264.7 Hz), 124.4 (d, J_{CF}=17.5 Hz), 118.6, 55.9, 42.0, 37.1, 36.4, 36.0, 28.9 and 28.0 ppm; ¹⁹F-NMR (CDCl₃) δ −119.3 (s, 2F) ppm; GC-MS (CI/CH₄), m/z 278 (MH⁺); Anal. Calcd for C₁₅H₁₃F₂NO₂: C, 64.98; H, 4.73 and N, 5.05. Found: C, 64.71; H, 4.59 and N, 5.08.
2-Endo-(bicyclo[2.2.1]heptan-2-yl)-4,7-difluoroisoindoline-1,3-dione (22): White crystals (13.0%, chromatography from CH₃OH/CH₂Cl₂); mp 93.5-94.5° C.; ¹H-NMR (CDCl₃) δ 7.35 (s, 2H), 4.44-4.40 (m, 1H), 4.16-4.09* (m, 0.1H), 2.54 (s, 1H), 2.51-2.39 (m, 2H), 2.26-2.16* (m, 0.19H), 1.76 (t, J=10.2 Hz, 2H), 1.54-1.29 (m, 5H) and 1.28-1.15* (m, 0.21H) ppm; ¹³C-NMR (CDCl₃) δ 165.2, 153.5 (d, J_{CF}=264.4 Hz), 124.5 (d, J_{CF}=17.6 Hz), 118.6 (d, J_{CF}=7.6 Hz), 55.9*, 55.2, 42.3, 42.0*, 37.9, 37.4, 37.1*, 36.4*, 36.0*, 28.8, 27.9 and 23.4 ppm; ¹⁹F-NMR (CDCl₃) δ −119.3 (s, 2F) ppm; GC-MS (CI/CH₄), m/z 277 (M⁺); Anal. Calcd for C₁₅H₁₃F₂NO₂: C, 64.98; H, 4.73; F, 13.70 and N, 5.05. Found: C, 64.85; H, 4.62; F, 13.44 and N, 5.00. [*The data came from the 2-exo-diastereomer of 22. 2-Chiral starting material of 22 came from 2-aminonorbornane hydrochloride produced by Sigma-Aldrich Company and it also showed out similar minor 2-exo-aminonorbornane hydrochloride diastereomer in its ¹H-NMR (in solvent DMSO-d₆)]. The NMR data showed compound 22 was primarily 2-endo diastereomer.

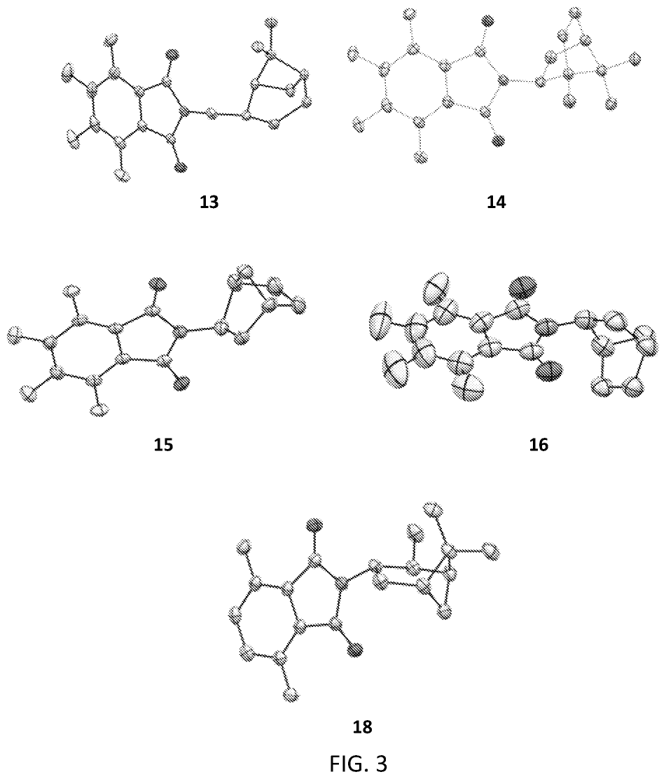

X-ray crystallographic structures for compounds 13-16 and 18 are shown in FIG. 3.

### Example 2

### Evaluation of Monoterpenoid Halophthalimides

### Methods

Assessments of drug anti-TNF-α and nitric oxide generation in RAW 264.7 cells: RAW 264.7 cells were purchased from ATCC Manassas, VA, USA). The cells were grown in DMEM media (Invitrogen, DMEM, high glucose, GlutaMAX Supplement, pyruvate, #10569-010) supplemented with 10% FBS (Invitrogen, Fetal Bovine Serum, qualified, heat inactivated, #16140-071) with penicillin and streptomycin (Invitrogen, Penicillin-Streptomycin (10,000 U/mL), #15140-122) and were maintained at 37 C and 5% CO2. The cells were grown to 75%-85% confluence on 10 cm plates (corning-Fal-con, #353003) following the protocol described by ATTC. to assess for drug effects on LPS-induced markers of inflammation, namely TNF-α protein and nitrite ion levels (NO₂, a stable end-product of nitric oxide metabolism) cells were seeded into 24 well (VWR, #10062-896) at a density of 200×103 cells per well. On each plate there was one blank well where no cells were added yet culture media in the well was treated similarly to wells with cells (n=1). The blank well media was used for background subtraction for various downstream assays. twenty-four hours after seeding the plate the seeding media was replaced with fresh media. Two hours after this the cells were exposed to a range of concentrations of drug test-compound(s) dissolved in 100% tissue culture grade dimethyl sjloxide (DMSO, SIGMA #D2650). Drug stocks were prepared in a 200 times more concentrated stock in DMSO. The final concentration of DMSO added to the cells was 0.5%. The effects of each concentration of drug were assessed in 4 wells per concentration in the 24 well plate (n=3 #-4). On each plate one set of wells were assigned as drug-vehicle control (i.e. DMSO), one test compound was assessed on one 24 well plate. The drug concentration used for each test agent were 1, 10 and 30 μM and are indicated in the associated figures. One hour after the drugs were added the cells were challenged with lipopolysaccharide (LPS, SIGMA, serotype 055:B5) at a final concentration of 60 ng/ml. Twenty to twenty-four hours after the cells were challenged with LPS the culture media was collected and utilized for the measurement of markers of inflammation.

Enzyme-linked immunosorbent assay for TNF-α protein: Media TNF-α levels were measured by use of the Biolegend ELISA MAX Set Delux ELISA (#430904). The protocol used was that recommended by the manufacturer. In brief, a day prior to performing the assay, a 96 well plate was coated with a capture antibody directed against TNF-α. The following day the assay plate was washed and blocked with the kit blocking agent for 1 h while being mixed on a plate shaker at 200 rpm (all incubations were mixed at 200 rpm unless stated otherwise). A TNF-α protein standard curve was prepared in the assay diluent following the kit instructions and unknown media samples were likewise diluted in the same assay diluent. After blocking, the plate was washed and then both the standards and unknown media samples were added to the plate in duplicate. The standards and unknown samples were incubated for 2 h. After the incubation, the plate was washed and then the biotin labeled detection antibody was added to each well and incubated for a further 1 h. After this incubation, the avidin-HRP conjugate complex was added to each well and incubated for 30 min. The plate was washed and the chromogenic substrate 3,3′,5,5′-tetramethylbenzidine solution was added to each well. At this point the plate was covered and incubated in the dark for 15 min, with no mixing. After 15 min the chromogenic reaction was stopped by the addition of 2 N H₂SO₄ and the absorbance was read at 450 nm and for background subtractions at 570 nm, on a SPECTRAmax PLUS plate reader. The absorbances were used to generate a TNF-α a protein standard curve and the protein levels in the unknown samples were determined using SoftMax Pro V5, Molecular Devices.

Nitrite ion detection in drug treated RAW 267.4 cell culture media: To measure the levels of nitrite ion NO₂—) the Griess Reagent System (Promega, Madison, WI, Cat #G2930) was utilized. The protocol followed was that recommended by the manufacturer. In brief a NO₂— ion standard curve was prepared in culture media of the same composition to that used for the cell culture study. The concentration range of the standard curve was from 1.5 M to 100 M. Equal volumes (50 μL) of standards and unknown samples were added to clear 96 well plates in duplicate. Then 50 μL of sulfanilamide solution was added to each well and the plate was covered and incubated in the dark for 10 min. After the incubation period 50 μL of N-1-napthylethylenediamine dihydrochloride solution was added to each well and the plate was incubated in dark for a further 10 min. After this incubation, the plate was read at 550 nm on a SPECTRAmax PLUS plate reader and the absorbances were used to generate a NO₂— standard curve and the NO₂— ion levels of the unknown samples are determined using SoftMax Pro V5, Molecular Devices.

Cellular viability assessment of drug treated RAW 264.7 cells: RAW 264.7 cell viability was determined by use of the CellTiter 96 AQueous One Solution Cell Proliferation Assay (Promega, Madison, WI). Changes in cellular health status were determined by use of indirect measures related to the formation of a colored tetrazolium dye product that can be measured spectrophotometrically at 490 nm. An increase in absorbance at 490 nm is indicative of an increase in numbers of healthy cells; similarly a reduction in absorbance is indicative of a reduction of healthy cells. After the culture media was harvested for use in TNF-α and NO₂— ion assays, the media was replaced with 0.5 mL of fresh media plus 100 μL of the cell viability reaction mixture. After a 20-30 min incubation at 37° C. with 5% CO2 the absorbance at 490 nm was obtained using an infinite M200 PRO plate reader (TECAN, USA).

Statistical analyses for RAW-derived assays: Data are expressed as a percentage change from the DMSO-vehicle control measurements. Measurements are expressed as mean±standard errors, where the n number represents the number of wells in a 24 well plate. Due to the presence of one blank control well on each plate, for some drug concentrations the n number is 3 # and not 4. Statistical comparisons were undertaken by use of a One-Way ANOVA with appropriate Bonferroni corrections for multiple comparisons, as required (GraphPad InStat Version 3.05). P values of <0.05 are considered to be of statistical significance.

### Nitrite and TNF-α Inhibitory Activities

Most neurons are highly vulnerable to RNS and ROS. Nitric oxide (NO) is one of RNSs, which is very short lived and cannot be readily measured. However, nitrite is a stable end product of nitric oxide metabolism. Therefore, nitrite acts as a surrogate measure of NO. The Griess Reagent System (Promega Corporation, Madison, WI) can be applied to measure nitrite levels in culture media following the manufacturer's protocol, and this system was used in the present study as it is a more sensitive assay compared to quantifying actions on TNF-α by ELISA assay. RAW 264.7 cells possessing features of microglial cell were challenged by lipopolysaccharide (LPS)-induced inflammation (LPS: 30 μg/mL. Sigma, St Louis, MO:serotype 055:B5). This challenge induces high level of TNF-α protein accompanied by a robust generation of nitrite.

The novel fluorophthalimides containing monoterpenoids 11-22 were evaluated to determine their effects on the levels of nitrite, TNF-α generated and released at 24 hours (Tweedie et al., Open BioChem J. 2011, 5:37). The summary data relating to activities on their LPS-induced TNF-α, nitrite and cell health is shown in Table 6.

TABLE 6
Inhibition of LPS-induced nitrite and TNF-α production in RAW 264.7
cells, cell viability and calculated lipophilicity of assayed compounds 11-22.

| Compound | | Nitrite | | TNF-α | | Cell Viability | | |
|---|---|---|---|---|---|---|---|---|
| # | μM | % Δ | P | % Δ | P | % Δ | P | ClogD |
| 11 | 1 | 48 ± 2 | <0.0000 | 76 ± 2 | 0.0004 | 90 ± 4 | 0.1364 | 3.56 |
| 12 | 1 | 24 ± 3 | <0.0000 | 56 ± 5 | 0.0006 | 63 ± 4 | 0.0005 | 3.56 |
| 13 | 1 | 31 ± 3 | <0.0000 | 62 ± 3 | 0.0006 | 78 ± 3 | 0.0036 | 3.69 |
| 14 | 1 | 26 ± 3 | <0.0000 | 55 ± 1 | 0.0000 | 95 ± 4 | 0.3097 | 3.43 |
| 15 | 1 | 25 ± 2 | <0.0000 | 71 ± 2 | 0.0003 | 108 ± 6 | 0.2749 | 3.04 |
| 16 | 1 | 36 ± 4 | <0.0000 | 74 ± 3 | 0.0102 | 94 ± 1 | 0.0318 | 3.04 |
| 17 | 1 | 90 ± 5 | 0.2145 | 95 ± 3 | 0.2450 | 100 ± 4 | 0.9667 | 3.24 |
| | 10 | 54 ± 3 | 0.0002 | 77 ± 3 | 0.0012 | 81 ± 5 | 0.0230 | |
| 18 | 1 | 96 ± 4 | 0.5187 | 101 ± 5 | 0.8280 | 102 ± 4 | 0.7025 | 3.24 |
| | 10 | 71 ± 4 | 0.0020 | 89 ± 5 | 0.2058 | 76 ± 5 | 0.0096 | |
| 19 | 1 | 79 ± 2 | 0.0664 | 102 ± 2 | 0.5291 | 98 ± 2 | 0.3369 | 3.37 |
| | 10 | 63 ± 3 | 0.0101 | 82 ± 2 | 0.0029 | 93 ± 4 | 0.1870 | |
| | 30 | 22 ± 1 | 0.0002 | not measured | | 67 ± 3 | 0.0001 | |
| 20 | 1 | 90 ± 2 | 0.1269 | 100 ± 2 | 0.9035 | 101 ± 3 | 0.8594 | 3.11 |
| | 10 | 78 ± 3 | 0.0147 | 98 ± 3 | 0.7224 | 98 ± 4 | 0.6631 | |
| | 30 | 39 ± 5 | 0.0004 | 76 ± 4 | 0.0071 | 96 ± 6 | 0.6173 | |
| 21 | 1 | 98 ± 7 | 0.8279 | 90 ± 1 | 0.0408 | 95 ± 3 | 0.1788 | 2.72 |
| | 10 | 127 ± 2 | 0.0007 | 90 ± 1 | 0.0428 | 95 ± 4 | 0.2730 | |
| | 30 | 83 ± 10 | 0.2095 | not measured | | 70 ± 6 | 0.0085 | |
| 22 | 1 | 103 ± 5 | 0.5967 | 102 ± 5 | 0.7495 | 99 ± 3 | 0.9082 | 2.72 |
| | 10 | 104 ± 2 | 0.2062 | 87 ± 5 | 0.0903 | 85 ± 2 | 0.0138 | |

| | | | | | |
|---|---|---|---|---|---|
| 30 | 50 ± 3 | <0.0000 | not measured | 53 ± 5 | 0.0006 |

Values are mean ± S.E.M. of n = 3 measurements. A students t-test was used to assess for statistically significant changes; P < 0.05 was considered significant. ClogD values were determined at pH 7.0 (CompuDrug, Pallas).
a. Compounds were screened respectively at 1 μM, 10 μM and 30 μM for nitrite, TNF-α and cell viability assays.
b. Percentage of vehicle control value; vehicle control = 100% analyte.

At just 1 μM, compounds 14, 15 and 16 were not only able to significantly reduce the nitrite levels (25-36%) but also lower TNF-α level (55-74%). Furthermore, they had very high cell viability (≥94%). In other words, they are low cellular toxicity or noncytotoxic at 1 μM. At 30 μM, compound 20 possessed good nitrite inhibitory activity (39%) and medium anti-TNF-α activity (76%), and at this concentration its cellular toxicity was still very low (cell viability 96%).

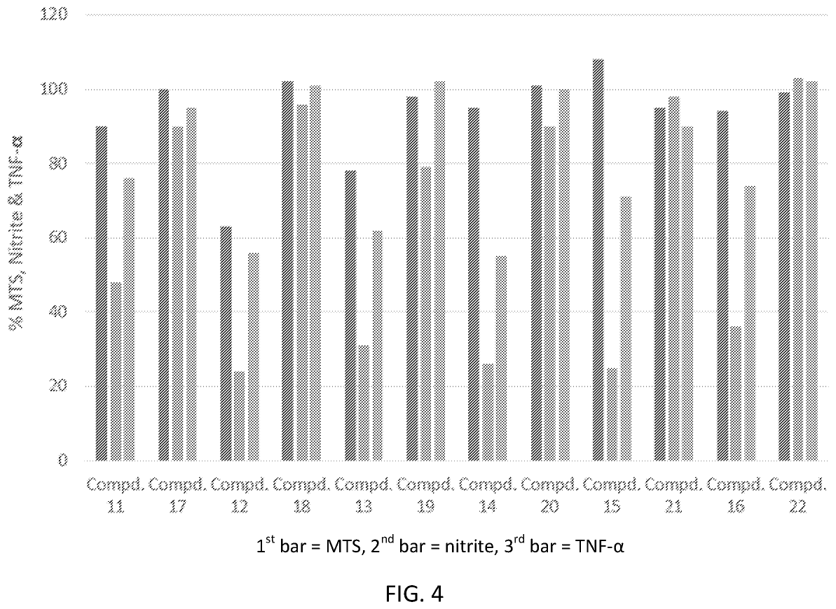

FIG. 4 showed that at 1 μM, six pairs of tetrafluorophthalimides and difluorophthalimides containing monoterpenoids, 11-17, 12-18, 13-19, 14-20, 15-21 and 16-22, had obviously different nitrite inhibitory activities in each pair. 11, 12, 13, 14, 15 and 16 possess more potent nitrite inhibitory activity than that of corresponding 17, 18, 19, 20, 21 and 22. It meant that the anti-nitrite activity of the tetrafluorophthalimides were much more potent than that of corresponding difluorophthalimides.

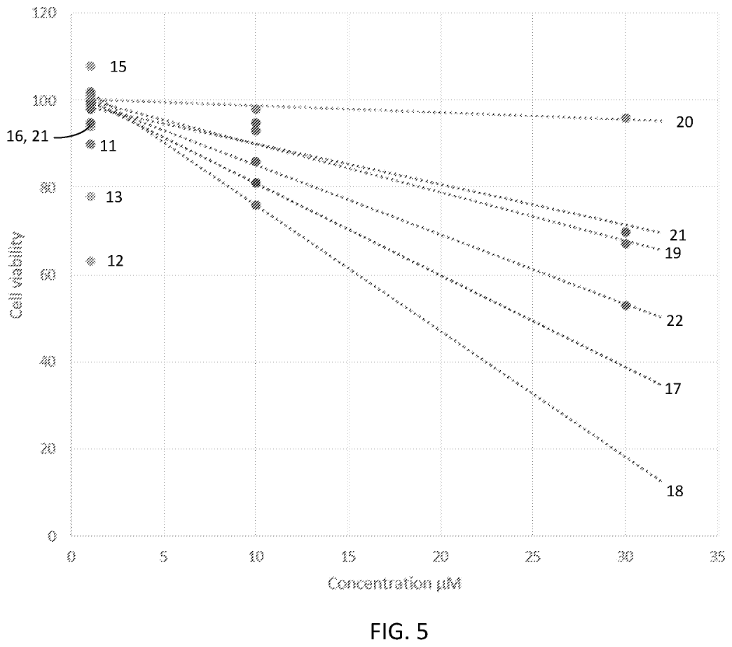

With increasing drug concentration, anti-nitrite and anti-TNF-α activities of candidates were generally increased, but their cellular toxicity was also increased. FIG. 5 shows that the cell viability of compound 20 was slowest to decrease (the smallest slope absolute value) with its concentration from 1 to 30 μM. That is, the cytotoxicity of N-bornyl difluorophthalimide 20 was the smallest in the assessment system of anti-nitrite and anti-TNF-α.

SAR analysis showed three interesting points. First, with respect to the phthalimide moiety, tetrafluoro-substituted phthalimide derivatives are much more potent to lower nitrite and TNF-α levels at 1 μM than the corresponding difluoro-substituted phthalimide derivatives. Namely, the more fluorinated aromatic ring provided more potent anti-nitrite and anti-TNF-α activity of the corresponding fluorophthalimide substituted by bridged ring. Secondly, with respect to the bridged ring moiety, at 1 μM N-bornyl and N-norbornyl tetrafluorophthalimides have lower cellular toxicity ensuring good biological activities. Thirdly, with respect to stereoisomeric differences, the cytotoxicity of enantiomer (1R,2R,3R,5S)-isopinocampheyl tetrafluorophthalimide 11 was lower than that of (1S,2S,3S,5R)-isopinocampheyl tetrafluorophthalimide 12. The nitrite inhibitory activity of N-exo-2-norbornyltetrafluorophthalimide 15 was slightly more potent than diastereomer 16. The anti-TNF-α activity of compound 15 was similar to diastereomer 16. However, the cytotoxicity of 15 was slightly lower than diastereomer 16. In order to make good use of these novel candidate agents possessing potent biological activity, balancing biological activity and cytotoxicity may be an important consideration.

### Anti-SARS-CoV-2 Activity

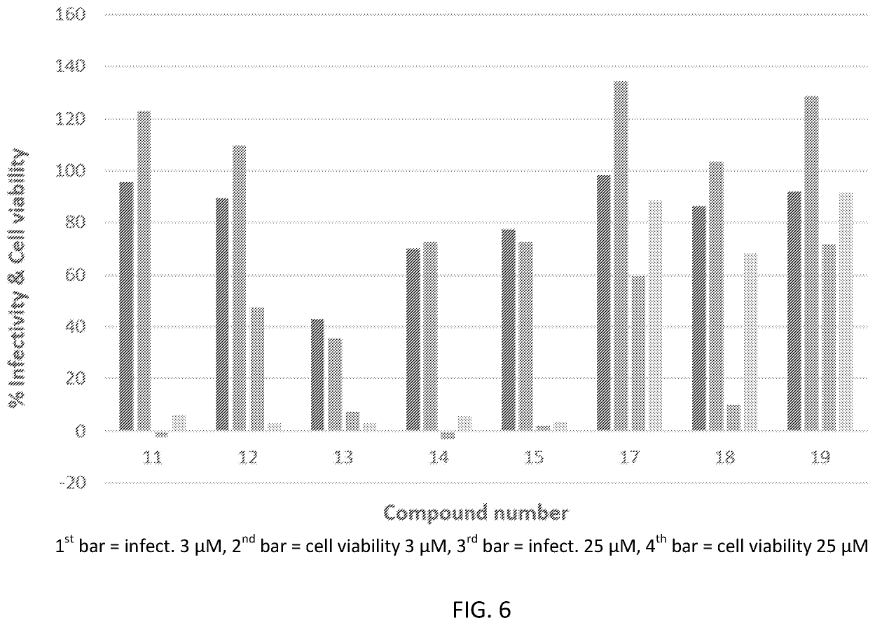

Anti-SARS-CoV-2 activity and cytotoxicity of compounds 11, 12, 13, 14, 15, 17, 18 and 19 are shown in FIG. 6. Compound 18 exhibited potent SARS-CoV-2 inhibitory activity, and it also presented preliminarily acceptable cytotoxicity at 25 μM.

### CONCLUSION

Twelve novel compounds possessing N-isopinocampheyl, N-bornyl, N-norbornyl and N-myrtanyl fluorophthalimides were synthesized. Comprehensive biological assessments determined that the compounds 14, 15 and 16 were promising for anti-inflammatory drug candidates, since they had potent nitrite inhibitory activity at a very low noncytotoxic concentration (e.g. 1 μM). This means that in the treatment of neurodegenerative diseases (such as Alzheimer's disease etc.), those very low doses of highly active drugs can be used. Compound 18 was worthy of further investigation for anti-SARS virus. Compound 20 had both better anti-nitrite activity and low cellular toxicity at 30 μM. SAR analysis shows that combining some monoterpenoids with fluorophthalimides may provide novel synthetic anti-inflammatory and anti-viral drugs.

### Example 3

### Synthesis of Thionated Aminofluorophthalimides

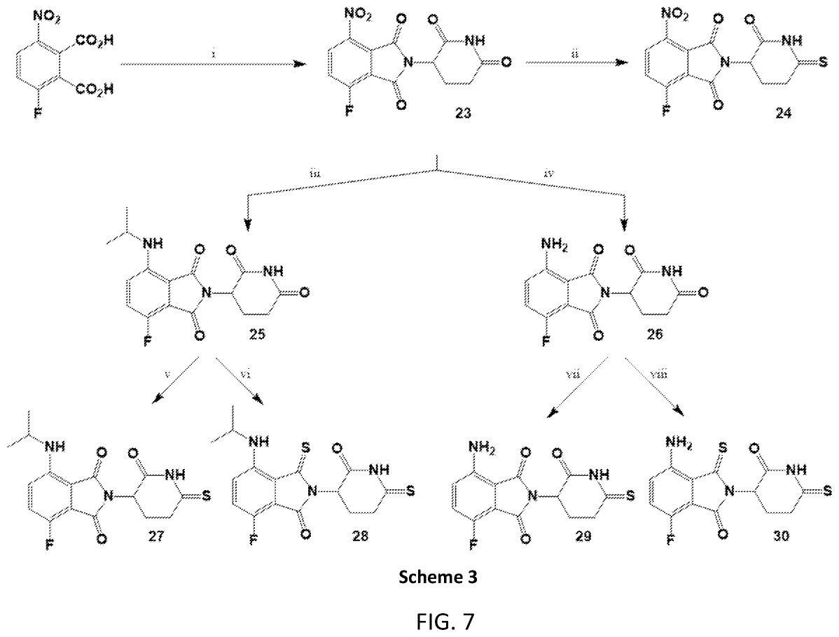

Initially, a collection of 7-fluoropomalidomides with all consequent materials stemming from an initial condensation of 3-aminoglutatrimide with the readily available 3-fluoro-6-nitrophthalic acid 7 (Hurth et al., Tetrahedron Lett 2015, 56:2860-2862) as shown in Scheme 3 (FIG. 7). General Conditions: (i) 3-aminopiperidine-2,6-dione, AcOH, 130° C., 16 hr. (ii) Lawesson's Reagent, 1,4-dioxane, reflux, overnight; 33% yield. (iii) H₂ (50 psi), PtO₂ (cat), 3:1 acetone/AcOH, H₂SO₄ (cat); 58% yield. (iv) H₂ (50 psi), 10% Pd/C (cat), H₂SO₄ (cat), AcOH; 61% yield. (v) Lawesson's Reagent, toluene, 105° C., 24 hr; 46% yield. (vi) P₄S₁₀-pyridine complex, 1,4-dioxane, 100° C., 40 hr; 36% yield. (vii) Lawesson's Reagent, 1,4-dioxane, reflux, 20 hr; 29% yield. (viii) P₄S₁₀-pyridine complex, 1,4-dioxane, 100° C., 20 hr; 29% yield.

The resulting fluorinated nitrothalidomide 23 proved a valuable springboard, permitting access to three distinct branches of our main synthetic tree. For example, monothionated 24 was obtained by a direct thionation with Lawesson's reagent, while careful control of reduction conditions provided access to the fluorinated pomalidomide and N-alkylpomalidomide cohorts. In this regard, reductive amination over PtO₂ in an acetone/acetic acid yielded isopropylated analog 25, while simple hydrogenation over Pd/C yielded primary amino compound 26. Subsequently, and for each corresponding pomalidomide analog, monothionation at the 6′-glutrarimide carbonyl was effected with Lawesson's reagent, while 3,6′-dithionation was achieved using P₄S₁₀-pyridine in 1,4-dioxane (Scerba et al., Synlett 2021, 32:917-922).

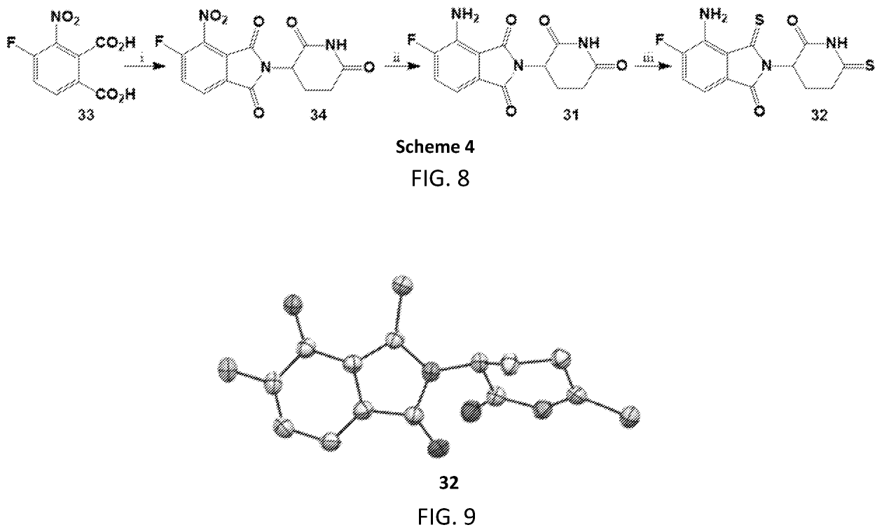

A series of 5-fluorophthalimides were prepared as shown in Scheme 4 (FIG. 8). General conditions: (i) 3-aminopiperidine-2,6-dione, AcOH, 130° C., 16 hr; 78% yield (ii) H₂ (50 psi), 10% Pd/C (cat), AcOH (cat); 50% yield. (iii) P₄S₁₀-pyridine complex, 1,4-Dioxane, 100° C., 18 hr; 31% yield.

Full details for the syntheses and characterizations are provided below.

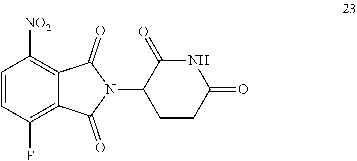 [SMILES: O=C1CCC(N2C(=O)c3c(F)ccc([N+](=O)[O-])c3C2=O)C(=O)N1]

2-(2,6-dioxopiperidin-3-yl)-4-fluoro-7-nitroisoindoline-1,3-dione (23) A 1000 mL round bottom flask was charged with 3-aminopiperidine-2,6-dione hydrochloride (35.9 g, 0.22 mol. 1.0 eq) and a stirring bar. The material was suspended in 600 mL MeOH and treated with triethylamine (33.4 mL, 0.24 mol, 1.1 eq). The mix was stirred and warmed gently to 40° C. for about 5 minutes, after which the heating was removed, and the reaction was stirred at room temperature for 1 hour. The solvents were removed, and the resulting residue was re-dissolved in 650 mL of acetic acid. Then, 3-fluoro-6-nitrophthalic acid (Hurth et al., Tetrahedron Lett 2015, 56:2860-2862) (49.94 g, 0.22 mol, 1.0 eq) was added and the mixture was heated to 130° C. for 16 hours. After cooling the mixture to room temperature, the resulting slurry was poured over crushed ice and allowed to stand overnight. The resulting precipitate was filtered off, dried, washed with portions of cold acetone and ether, and finally dried in vacuo to give a grey/purple powder that was used directly in the next reaction without further purification. ¹H NMR (400 MHz, DMSO-d₆) d 11.17 (s, 1H), 8.44 (dd, J=9.3, 3.4 Hz, 1H), 7.99 (t, J=8.7 Hz, 1H), 5.20 (dd, J=12.9, 5.2 Hz, 1H), 2.98-2.79 (m, 1H), 2.67-2.40 (m, 2H), 2.15-1.93 (m, 1H). ¹³C NMR (101 MHz, DMSO-d₆) δ 172.61, 169.23, 162.09, 161.52 (d, J=1.8 Hz), 158.17 (d, J=268.0 Hz), 141.04 (d, J=3.3 Hz), 132.40 (d, J=9.6 Hz), 125.32 (d, J=2.2 Hz), 124.61 (d, J=21.9 Hz), 118.81 (d, J=14.4 Hz), 49.49, 30.80, 21.61. ¹⁹F NMR (377 MHz, DMSO-d₆) d −107.80 (d, J=7.0 Hz). HRMS (ESI) calc for [C₁₃H₈FO₆N₃+K]⁺ 360.0029, found 360.0029. Samples suitable for biological analyses were prepared by recrystallization from EtOAc/hexanes, yielding a light tan-grey solid. Elemental analysis: Anal. Calcd for C₁₃H₈FO₆N₃: C, 48.61; H, 2.51; N, 13.08. Found C, 48.60; H, 2.54; N, 13.05.

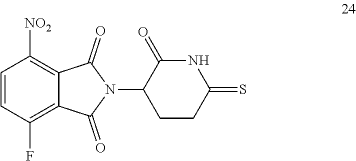 [SMILES: O=C1NC(=S)CCC1N1C(=O)c2c(F)ccc([N+](=O)[O-])c2C1=O]

4-fluoro-7-nitro-2-(2-oxo-6-thioxopiperidin-3-yl) isoindoline-1,3-dione (24) Compound 23 (500 mg, 1.48 mmol, 1.0 eq) was combined with Lawesson's Reagent (500 mg, 1.24 mmol, 0.85 eq) in 40 mL of dioxane and heated to reflux. The reaction was monitored by LC/MS. After stirring overnight, the reaction was cooled to RT and evaporated to dryness. The residue was combined with Ethyl Acetate and extracted with water. The combined organics were dried over MgSO₄ and evaporated. The resulting residue was purified by prep HPLC to give 171 mg of a pale-yellow solid, 33% yield. ¹H NMR (400 MHz, DMSO-d₆) δ 12.62 (s, 1H), 8.43 (dd, J=9.0, 3.5 Hz, 1H), 7.94 (t, J=8.7 Hz, 1H), 5.34 (dd, J=12.8, 5.6 Hz, 1H), 3.31-3.17 (m, 2H), 2.53-2.38 (m, 1H, partially obscured), 2.16-2.02 (m, 1H). ¹³C NMR (101 MHz, DMSO-d₆) δ 210.39, 166.44, 161.96, 161.40 (d, J=1.7 Hz), 158.15 (d, J=268.1 Hz), 141.02 (d, J=3.3 Hz), 132.42 (d, J=9.5 Hz), 125.31 (d, J=2.2 Hz), 124.61 (d, J=21.9 Hz), 118.81 (d, J=14.3 Hz), 49.26, 40.58, 23.26. ¹⁹F NMR (377 MHz, DMSO-d₆) δ −107.72 (dd, J=8.7, 3.8 Hz). HRMS (ESI) calc for [C₁₃H₈O₅N₃FS+H]⁺, 338.0241, found 338.0258. Samples suitable for biological analyses were prepared by recrystallization from acetonitrile, yielding a pale-yellow powder. Elemental analysis: Anal. Calcd for C₁₃H₈O₅N₃FS: C, 46.29; H, 2.39; N, 12.46; S, 9.51. Found C, 46.11; H, 2.31; N, 12.22; S, 9.28.

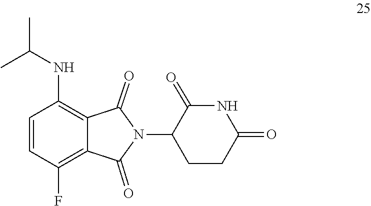 [SMILES: CC(C)Nc1ccc(F)c2c1C(=O)N(C1CCC(=O)NC1=O)C2=O]

2-(2,6-dioxopiperidin-3-yl)-4-fluoro-7-(isopropylamino) isoindoline-1,3-dione (25) In a Parr flask, compound 23 (2.0 g, 6.2 mmol, 1 eq) was dissolved in 150 mL of 3:1 acetone/acetic acid solution. A small scoop of PtO₂ was added, followed by a few drops of concentrated sulfuric acid, and the mixture was hydrogenated under ˜50 psi pressure until consumption ceased. The resulting yellow-green mixture was filtered through celite, and the resulting liquor was evaporated under reduced pressure. The resulting residue was dissolved in ethyl acetate and extracted with water, saturated NaHCO₃ and finally brine. The organics were dried over Na₂SO₄ and evaporated to give a crude brown/green solid which was recrystallized from EtOAc/Hex to give 1.2 g, 58% yield of compound 10. ¹H NMR (400 MHz, DMSO-d₆) δ 11.10 (s, 1H), 7.48 (t, J=9.1 Hz, 1H), 7.19 (d, J=9.7 Hz, 1H), 6.12 (d, J=8.0 Hz, 1H), 5.05 (dd, J=12.8, 5.3 Hz, 1H), 3.86 (m, 1H), 2.92-2.80 (m, 1H), 2.6 (m, 2H-partially obscured by DMSO), 2.17-1.83 (m, 1H), 1.21 (d, J=6.2 Hz, 6H). ¹³C NMR (101 MHz, DMSO-d₆) δ 172.74, 169.87, 168.13 (d, J=3.1 Hz), 163.90, 147.91 (d, J=251.8 Hz), 142.84, 125.46, 120.75 (d, J=6.3 Hz), 116.13 (d, J=12.7 Hz), 108.22, 48.64, 43.35, 30.91, 22.37, 21.99. ¹⁹F NMR (377 MHz, DMSO-d₆) δ −129.70 (d, J=8.8 Hz). HRMS (MALDI) calc for [C₁₆H₁₆FO₄N₃+H]⁺ 334.1198, found 334.1195. Samples suitable for biological analyses were prepared by recrystallization from acetonitrile, yielding a green-yellow solid. Elemental analysis: Anal. Calcd for C₁₆H₁₆FO₄N₃: C, 57.66; H, 4.84; N, 12.61. Found C, 57.67; H, 4.77; N, 12.67.

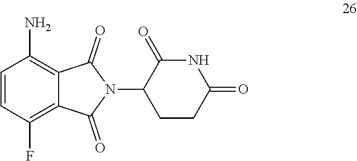 [SMILES: Nc1ccc(F)c2c1C(=O)N(C1CCC(=O)NC1=O)C2=O]

4-amino-2-(2,6-dioxopiperidin-3-yl)-7-fluoroisoindoline-1,3-dione (26) A portion of compound 23 (10.0 g, 0.031 mol) was combined with 500 mg of 10% Pd/C and a few drops of sulfuric acid in 200 mL of acetic acid. The mixture was hydrogenated until consumption stopped, leaving a heterogenous mixture that is filtered over celite. The remaining residue was washed with portions of acetic acid. The resulting combined yellow filtrate is poured over ˜2 L of crushed ice and left to stand overnight. The resulting yellow precipitate is filtered off and washed with water. The filter cake is left to dry, giving a thick semi-solid paste which is transferred to a large flask, washed with cold acetone, and finally co-evaporated three times with toluene to yield 5.6 g of a free-flowing yellow powder, 61% yield. ¹H NMR (400 MHz, DMSO-d₆) d 11.09 (bs, 1H), 7.37 (t, J=8.9 Hz, 1H), 7.07 (d, J=9.3 Hz, 1H), 6.42 (s, 2H), 5.33-4.89 (m, 1H), 2.88 (t, J=15.8 Hz, 1H), 2.65-2.41 (m, 2H), 2.18-1.93 (m, 1H). ¹³C NMR (101 MHz, DMSO-d₆) δ 172.76, 169.94, 167.56 (d, J=2.9 Hz), 164.07, 148.21 (d, J=251.8 Hz), 143.87, 124.95 (d, J=9.2 Hz), 124.81 (d, J=6.9 Hz), 115.45 (d, J=12.1 Hz), 107.50, 48.58, 30.94, 21.99. ¹⁹F NMR (377 MHz, DMSO-d₆) d −128.90 (d, J=8.5 Hz). HRMS (ESI) calc for [C₁₃H₁₀FO₄N₃+Na]⁺ 314.0548 found 314.0544. Samples suitable for biological analyses were prepared by recrystallization from acetonitrile, yielding a brilliant yellow solid. Elemental analysis: Anal. Calcd for C₁₃H₁₀FO₄N₃: C, 53.61; H, 3.46; N, 14.43. Found C, 53.58; H, 3.43; N, 14.50.

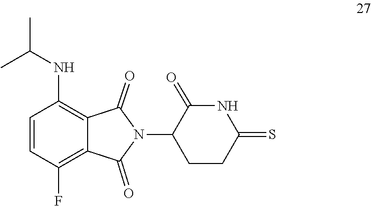 [SMILES: CC(C)Nc1ccc(F)c2c1C(=O)N(C1CCC(=S)NC1=O)C2=O]

4-fluoro-7-(isopropylamino)-2-(2-oxo-6-thioxopiperidin-3-yl) isoindoline-1,3-dione (27) Compound 25 (1.0 g, 3.0 mmol, 1.0 eq) was suspended in 100 mL toluene. Then, Lawesson's Reagent (0.484 g, 1.20 mmol, 0.4 eq) was added and the mixture was heated to 105° C. After 4 hr of reaction time, an additional portion of Lawesson's Reagent was added (0.484 g, 1.20 mmol, 0.4 eq) and the reaction was stirred overnight. After 24 hr of total reaction time, ˜90% of the starting material had been consumed, with the desired mono-thionated material being the major product among other thionated isomers as judged by LC/MS analysis. At this point, the reaction was cooled to RT and evaporated under reduced pressure. The residue was then combined with Ethyl Acetate and extracted with 1M HCl, followed by saturated NaHCO₃. After drying over MgSO₄, the liquor was evaporated to dryness and purified by prep HPLC to yield 0.492 g of a mustard-yellow powder, 46% yield. ¹H NMR (400 MHz, DMSO-d₆) d 12.63 (s, 1H), 7.48 (t, J=9.1 Hz, 1H), 7.19 (dd, J=9.4, 3.4 Hz, 1H), 6.12 (d, J=8.3 Hz, 1H), 5.17 (dd, J=12.8, 5.6 Hz, 1H), 3.86 (dt, J=8.2, 6.3 Hz, 1H), 3.28-3.07 (m, 2H), 2.58-2.39 (m, 1H, partially obscured), 2.15-1.94 (m, 1H), 1.21 (d, J=6.3 Hz, 6H). ¹³C NMR (101 MHz, DMSO-d₆) δ 210.50, 167.94 (d, J=3.1 Hz), 167.15, 163.76, 147.92 (d, J=252.4 Hz), 142.87, 125.41 (d, J=22.3 Hz), 120.85 (d, J=6.4 Hz), 115.98 (d, J=12.7 Hz), 108.11, 48.41, 43.36, 40.71, 23.62, 22.37. ¹⁹F NMR (377 MHz, DMSO-d₆) δ −129.56 (m, 1F). HRMS (ESI) calc for [C₁₆H₁₆O₃N₃FS+H]⁺, 350.0969, found 350.0987. Samples suitable for biological analyses were prepared by recrystallization from acetonitrile, yielding a mustard-green solid. Elemental analysis: Anal. Calcd for C₁₆H₁₆O₃N₃FS: C, 55.00; H, 4.62; N, 12.03; S, 9.18. Found C, 55.29; H, 4.76; N, 12.25; S, 9.04.

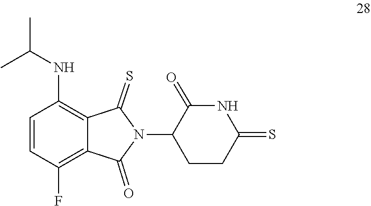 [SMILES: CC(C)Nc1ccc(F)c2c1C(=S)N(C1CCC(=S)NC1=O)C2=O]

7-fluoro-4-(isopropylamino)-2-(2-oxo-6-thioxopiperidin-3-yl)-3-thioxoisoindolin-1-one (28) Compound 25 (30 mg, 0.091 mmol, 1.0 eq) and PAS 10-pyridine complex (52 mg, 0.14 mmol, 1.5 eq) were charged into a vial along with a stir bar. After the vessel was sealed and placed under N₂, 2.5 mL of 1,4-dioxane was injected and the mixture was stirred and heated to 100° C. The reaction, initially cloudy yellow, proceeds to a homogenous clear orange solution and finally to a deep red oily mixture. After 20 hours, an additional small portion of P₄S₁₀-pyridine complex (17 mg, 0.05 mmol, 0.5 eq) is added, and the reaction is continued for another 20 hr. After roughly 40 hr of total reaction, all of the starting material had been consumed, with a large product peak as well as some minor monothionated species. At this point, the reaction was stopped and cooled to room temperature at which point a thick dark oily residue remains at the bottom of the reaction vial. The best workup from here branches into two parts (A and B) which ultimately are reunited in a final extraction protocol. (A) The red reaction liquor is decanted, evaporated to dryness, redissolved in ethyl acetate and set aside for later use. (B) The dark oily residue that remains in the original reaction flask is then attended to; it is combined with ethyl acetate and saturated sodium bicarbonate (NaHCO₃) and stirred gently at which point the thick oil fully dissolves and partitions into a red organic layer and a slightly cloudy aqueous layer. These layers are transferred to a separatory funnel and combined with the ethyl acetate crop from part A, resulting in a combined red organic layer perched upon a lower bicarbonate aqueous layer. This initial concoction is not shaken in the traditional manner due to emulsion formation; rather, the aqueous bicarbonate layer is simply drained off. After this point, an additional wash with saturated NaHCO₃ is performed in the traditional, shaken manner, followed by washings with water (1×), 0.5M HCl (1×) and finally brine (1×) before being dried over sodium sulfate. The organics are decanted and evaporated to dryness, leaving a dark red residue which in purified by prep-HPLC to yield 13 as a vibrant dark red powder, 11.9 mg, 36% yield (average of three runs). ¹H NMR (400 MHz, Chloroform-d) δ 9.39 (s, 1H), 8.57 (s, 1H), 7.21 (1, J=8.8 Hz, 1H), 7.03 (d, J=9.7 Hz, 1H), 5.65 (bs, 1H*), 3.84 (m, 1H), 3.50 (d, J=16.7 Hz, 1H), 3.09-2.71 (m, 2H), 2.10 (s, 1H), 1.34 (d, J=6.3 Hz, 6H). ¹⁹F NMR (377 MHz, Chloroform-d) δ −128.40. Note that the starred * signals are very broad and difficult to resolve even after increasing relaxation delays and scans. It is for these reasons that these NMR analyses are further supported with X-ray, HPLC and HRMS analyses. HRMS (ESI) calc for [C₁₆H₁₆FO₂N₃S₂+H]⁺, 366.0741, found 366.0726. Elemental analysis: Anal. Calcd for C₁₆H₁₆FO₂N₃S₂: C, 52.59; H, 4.41; N, 11.50; S, 17.55. Found C, 52.30; H, 4.34; N, 11.29; S, 17.49.

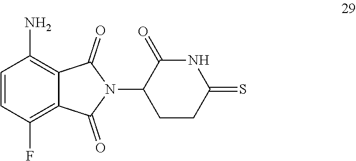 [SMILES: Nc1ccc(F)c2c1C(=O)N(C1CCC(=S)NC1=O)C2=O]

4-amino-7-fluoro-2-(2-oxo-6-thioxopiperidin-3-yl) isoindoline-1,3-dione (29) Compound 26 (500 mg, 1.72 mmol, 1.0 eq) and Lawesson's Reagent (416 mg, 1.03 mmol, 0.6 eq) were combined in 60 mL of dioxane and refluxed for 20 hr. The reaction was monitored by LC/MS, and after 20 hr, most of the starting material had been consumed. The reaction was cooled to RT, evaporated under reduced pressure and extracted repeatedly with Ethyl Acetate and water. The combined organics were dried over MgSO₄ and evaporated. The resulting residue was purified by prep HPLC to yield 156 mg of a yellow powder, 29% yield. ¹H NMR (400 MHz, DMSO-d₆) δ 12.62 (s, 1H), 7.37 (t, J=9.1 Hz, 1H), 7.07 (dd, J=9.2, 3.6 Hz, 1H), 6.45 (s, 2H), 5.17 (dd, J=12.8, 5.6 Hz, 1H), 3.19 (dd, J=11.1, 4.8 Hz, 2H), 2.50 (m, 1H, peak obscured), 2.07-1.99 (m, 1H). ¹³C NMR (101 MHz, DMSO-d₆) δ 210.54, 167.40 (d, J=3.0 Hz), 167.23, 163.94, 148.21 (d, J=252.0 Hz), 143.93, 125.01 (d, J=8.2 Hz), 124.87 (d, J=7.9 Hz), 115.40 (d, J=12.4 Hz), 107.40, 48.39, 40.76, 23.65. ¹⁹F NMR (377 MHz, DMSO-d₆) δ −128.74 (dd, J=9.1, 3.8 Hz). HRMS (ESI) calc for [C₁₃H₁₀O₃N₃FS+H]⁺, 308.0500, found 308.0512. Samples suitable for biological analyses were prepared by recrystallization from acetonitrile, yielding a bright yellow powder. Elemental analysis: Anal. Calcd for C₁₃H₁₀O₃N₃FS: C, 50.81; H, 3.28; N, 13.67; S, 10.43. Found C, 50.84; H, 3.21; N, 13.94; S, 10.36.

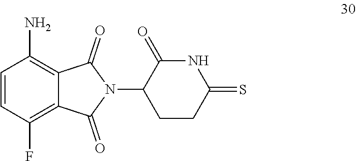 [SMILES: Nc1ccc(F)c2c1C(=O)N(C1CCC(=S)NC1=O)C2=O]

4-amino-7-fluoro-2-(2-oxo-6-thioxopiperidin-3-yl)-3-thioxoisoindolin-1-one (30) 7-fluoro-pomalidomide 26 (27 mg, 0.091 mmol, 1.0 eq) and P₄S₁₀-pyridine complex (52 mg, 0.14 mmol, 1.5 eq) were charged into a vial along with a stir bar. After the vessel was sealed and placed under N₂, 2.5 mL of 1,4-dioxane was injected and the mixture was stirred and heated to 100° C. The reaction, initially cloudy yellow, proceeds to a homogenous clear orange solution and finally to a deep red oily mixture. After 20 hours, the reaction is stopped and cooled to room temperature, at which point a thick dark oily residue remains at the bottom of the reaction vial. As before, the best workup from here branches into two parts (A and B) which ultimately are reunited in a final extraction procedure. (A) The red reaction liquor is decanted, evaporated to dryness, redissolved in ethyl acetate and set aside for later use. (B) The dark oily residue that remains in the original reaction flask is then attended to; it is combined with ethyl acetate and saturated sodium bicarbonate (NaHCO₃) and stirred gently at which point the thick oil fully dissolves and partitions into a red organic layer and a slightly cloudy aqueous layer. These layers are transferred to a separatory funnel and combined with the ethyl acetate crop from part A, resulting in a combined red organic layer perched upon a lower bicarbonate aqueous layer. This initial mixture is not shaken in the traditional manner due to emulsion formation; rather, the aqueous layer is simply drained off. After this point, an additional wash with saturated NaHCO₃ is performed in the traditional, shaken manner, followed by washings with water (1×), 0.5M HCl (1×) and finally brine (1×) before being dried over sodium sulfate. The organics were decanted and evaporated to dryness, leaving a dark red-orange residue which in purified by prep-HPLC to yield 15 as a bright orange-red powder, 8.6 mg, 29% yield (average of three runs). ¹H NMR (400 MHz, DMSO-d₆) δ 12.65 (bs, 1H) 7.9-7.54 (bs, 2H), 7.42 (d, J=8.3 Hz, 1H), 7.25 (bs, 1H), 5.85-5.40 (bm, 1H*), 3.24-2.78 (bm, 2.4H*), 2.01 (bs, 1H). ¹⁹F NMR (377 MHz, DMSO-d₆) δ −127.85 (bm, 1F*). Note that the starred * signals are very broad and difficult to resolve even after increasing relaxation delays and scans. It is for these reasons that these NMR analyses are further supported with X-ray, HPLC and HRMS analyses. HRMS: HRMS (MALDI) calc for [C₁₃H₁₀FO₂N₃S₂+Na]⁺ 346.00907, found 346.00931. Elemental analysis: Anal. Calcd for C₁₃H₁₀FO₂N₃S₂: C, 48.29; H, 3.12; N, 13.00; S, 19.83. Found C, 48.12; H, 3.12; N, 12.97; S, 19.68.

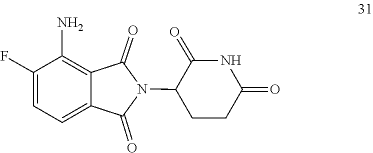 [SMILES: Nc1c(F)ccc2c1C(=O)N(C1CCC(=O)NC1=O)C2=O]

4-amino-2-(2,6-dioxopiperidin-3-yl)-5-fluoroisoindoline-1,3-dione (31) In a Parr flask, compound 34 (Scheme 4, FIG. 8) (545 mg, 1.70 mmol, 1.0 eq) was combined with 50 mL ethyl acetate and a small scoop of 10% Pd/C along with a few drops of acetic acid. The mixture was hydrogenated at 40 psi until consumption of H₂ ceased. The resulting mixture was filtered through Celite, and the resulting liquor was evaporated to dryness. The resulting residue was redissolved in ethyl acetate and extracted against saturated NaHCO₃, water, and finally brine. The organics were further dried with MgSO₄ before being evaporated. The resulting residue was recrystallized from acetonitrile to give 247 mg of a pale-yellow solid, 50% yield. ¹H NMR (400 MHz, DMSO-d₆) δ 11.11 (s, 1H), 7.44 (ddd, J=12.0, 7.8, 1.2 Hz, 1H), 7.05 (ddd, J=7.9, 3.7, 1.2 Hz, 1H), 6.57 (s, 2H), 5.06 (dd, J=12.5, 5.2 Hz, 1H), 2.88 (ddd, J=17.8, 13.6, 5.3 Hz, 1H), 2.65-2.50 (m, 2H, peak obscured), 2.03 (dd, J=10.4, 4.7 Hz, 1H). ¹³C NMR (101 MHz, DMSO-d₆) δ 172.76, 169.97, 167.45, 166.41, 154.01 (d, J=249.6 Hz), 135.79 (d, J=16.5 Hz), 127.36, 119.85 (d, J=19.4 Hz), 111.60, 111.35, 48.69, 30.94, 22.06. ¹⁹F NMR (377 MHz, DMSO-d₆) δ −124.86 (d, J=12.0 Hz). HRMS (MALDI) calc for [C₁₃H₁₀FN₃O₄+H]⁺ 292.07281, found 292.07350. Elemental analysis: Anal. Calcd for C₁₃H₁₀FN₃O₄: C, 53.61; H, 3.46; N, 14.43. Found C, 53.70; H, 3.47; N, 14.26.

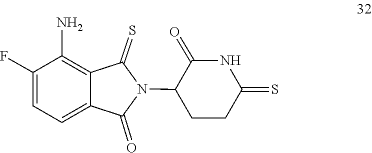 [SMILES: Nc1c(F)ccc2c1C(=S)N(C1CCC(=S)NC1=O)C2=O]

4-amino-5-fluoro-2-(2-oxo-6-thioxopiperidin-3-yl)-3-thioxoisoindolin-1-one (32) Compound 31 (100 mg, 0.34 mmol, 1.0 eq) and P₄S₁₀-pyridine complex (195 mg, 0.52 mmol, 1.5 eq) were charged into a vial along with a stir bar. After the vessel was sealed and placed under N₂, 10 mL of 1,4-dioxane was injected and the mixture was stirred and heated to 100° C. After 20 hours, the reaction is stopped and cooled to room temperature, at which point a thick dark oily residue remains at the bottom of the reaction vial. As before, the best workup from here branches into two parts (A and B) which ultimately are reunited in a final extraction procedure. (A) The red reaction liquor is decanted, evaporated to dryness, redissolved in ethyl acetate and set aside for later use. (B) The dark oily residue that remains in the original reaction flask is then attended to; it is combined with ethyl acetate and saturated sodium bicarbonate (NaHCO₃) and stirred gently at which point the thick oil fully dissolves and partitions into a red organic layer and a slightly cloudy aqueous layer. These layers are transferred to a separatory funnel and combined with the ethyl acetate crop from part A, resulting in a combined red organic layer perched upon a lower bicarbonate aqueous layer. This initial mixture is not shaken in the traditional manner due to emulsion formation; rather, the aqueous layer is simply drained off. After this point, an additional wash with saturated NaHCO₃ is performed in the traditional, shaken manner, followed by washings with water (1×), 0.5M HCl (1×) and finally brine (1×) before being dried over sodium sulfate. The organics are decanted and evaporated to dryness, leaving a dark red-orange residue which in purified by prep-HPLC to yield 17 as a bright orange-red powder, 34 mg, 31% yield. ¹H NMR (400 MHz, DMSO-d₆) δ 12.66 (s, 1H), 7.62 (s, 2H), 7.48 (dd, J=11.8, 7.8 Hz, 1H), 7.08 (s, 1H), 5.65 (m, *1H), 3.28-2.80 (m, *2H), 2.07-1.97 (m, 1H). ¹⁹F NMR (377 MHz, DMSO-d₆) δ −123.02 (d, J=10.2 Hz). HRMS (MALDI) calc for [C₁₃H₁₀FN₃O₂S₂+Na]⁺346.00907, found 346.00942. Note that the starred * signals are very broad and difficult to resolve even after increasing relaxation delays and scans. It is for these reasons that these NMR analyses are further supported with X-ray, HPLC and HRMS analyses.

Single Crystal X-Ray Crystallography: All reflection intensities were measured 110(2) K for compound 32, using a SuperNova diffractometer (equipped with Atlas detector) with Cu Kα radiation (λ=1.54178 Å) under the program CrysAlisPro (Version CrysAlisPro 1.171.39.29c, Rigaku OD, 2017). The same program was used to refine the cell dimensions and for data reduction. The structure was solved with the program SHELXS-2018/3 and was refined on F₂ with SHELXL-2018/3 (Sheldrick, Acta Cryst. 2015, C71: 3-8). Analytical numeric absorption correction using a multifaceted crystal model was applied using CrysAlisPro. The temperature of the data collection was controlled using the system Cryojet (manufactured by Oxford Instruments). The H atoms were placed at calculated positions (unless otherwise specified) using the instructions AFIX 13, AFIX 23 or AFIX 43 with isotropic displacement parameters having values 1.2 U_{eq} of the attached C atoms. The H atoms attached to N1 and N3 were found from difference Fourier maps, and their coordinates were refined pseudofreely using the DFIX instruction in order to keep the N—H bond distances within an acceptable range. The structure is ordered. CCDC/Deposition Number is 2182910. The data is shown in Table 7, and the X-ray crystallographic structure is shown in FIG. 9.

TABLE 7
Experimental X-Ray Details

Compound 32

Crystal data

| | |
|---|---|
| Chemical formula | C₁₃H₁₀FN₃O₂S₂ |
| Mᵣ | 323.36 |
| Crystal system, | Monoclinic, P2₁/n |
| space group | |
| Temperature (K) | 110 |
| a, b, c (Å) | 6.8284 (3), 11.5577 (5), 16.9699 (6) |
| β (°) | 96.332 (4) |
| V (Å³) | 1331.10 (10) |
| Z | 4 |
| Radiation type | Cu Kα |
| μ (mm⁻¹) | 3.83 |
| Crystal size (mm) | 0.07 × 0.06 × 0.04 |

Data collection

| | |
|---|---|
| Diffractometer | SuperNova, Dual, Cu at zero, Atlas |
| Absorption | Analytical CrysAlis PRO 1.171.41.93a (Rigaku Oxford |
| correction | Diffraction, 2020) Analytical numeric absorption correction |
| | using a multifaceted crystal model based on expressions |
| | derived by R. C. Clark & J. S. Reid. (Clark, R. C. & Reid, J. |
| | S. (1995). Acta Cryst. A51, 887-897) Empirical absorption |
| | correction using spherical harmonics, implemented in |
| | SCALE3 ABSPACK scaling algorithm. |
| Tₘᵢₙ, Tₘₐₓ | 0.839, 0.896 |
| No. of measured, | 10628, 2610, 2197 |
| independent and | |
| observed [I > | |
| 2σ(I)] reflections | |
| Rᵢₙₜ | 0.038 |
| (sin θ/λ)ₘₐₓ (Å⁻¹) | 0.616 |

Refinement

| | |
|---|---|
| R[F² > 2σ(F²)], | 0.059, 0.151, 1.16 |
| wR(F²), S | |
| No. of reflections | 2610 |
| No. of parameters | 199 |
| No. of restraints | 3 |
| H-atom treatment | H atoms treated by a mixture of independent and constrained |
| | refinement |
| Δρₘₐₓ, Δρₘᵢₙ (e Å⁻³) | 0.61, −0.35 |

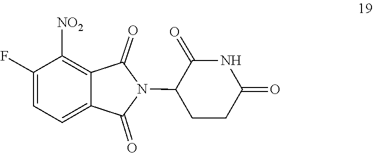 [SMILES: O=C1CCC(N2C(=O)c3ccc(F)c([N+](=O)[O-])c3C2=O)C(=O)N1]

2-(2,6-dioxopiperidin-3-yl)-5-fluoro-4-nitroisoindoline-1,3-dione (19) 3-aminopiperidine-2,6-dione hydrochloride (377 mg, 2.29 mmol, 1.05 eq) was suspended in 5 mL of methanol and treated with triethylamine (0.445 mL, 3.19 mmol, 1.0 eq). The mix was stirred and warmed gently to 40° C. for about 5 minutes, after which the heating was removed, and the reaction was stirred at room temperature for 30 minutes. Then, the solvents were removed under reduced pressure. The resulting solid mass was then combined with 5 mL acetic acid and 4-fluoro-3-nitrophthalic acid (US 2020/0061033 A1) (500 mg, 2.18 mmol, 1.0 eq) in a small sealed tube and heated to 130° C. overnight. After cooling to RT, water was added to precipitate a dark grey-black solid which was filtered off. An additional crop of product could be obtained by extracting the filtrate liquor with ethyl acetate. The combined crops provided 547 mg of product (78% yield) which was used directly in the next step without further purification.

### Example 4

### Evaluation of Thionated Aminofluorophthalimides

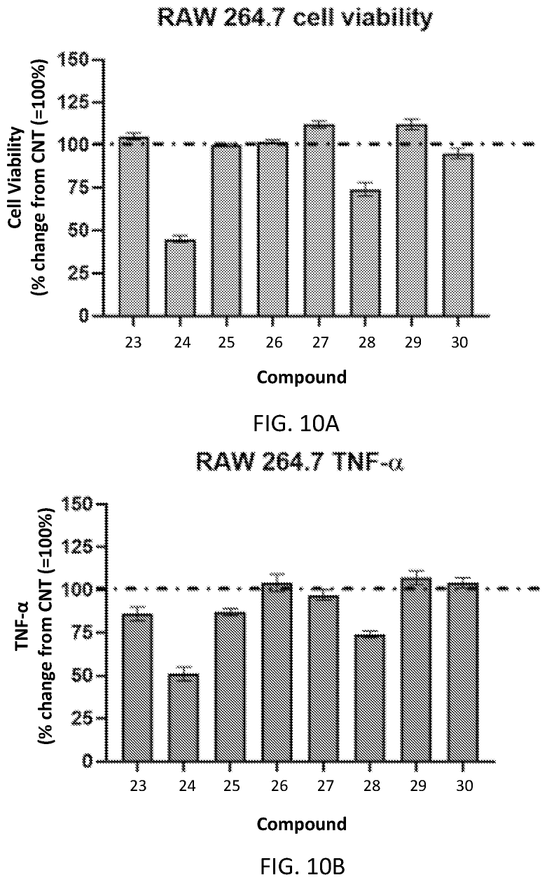

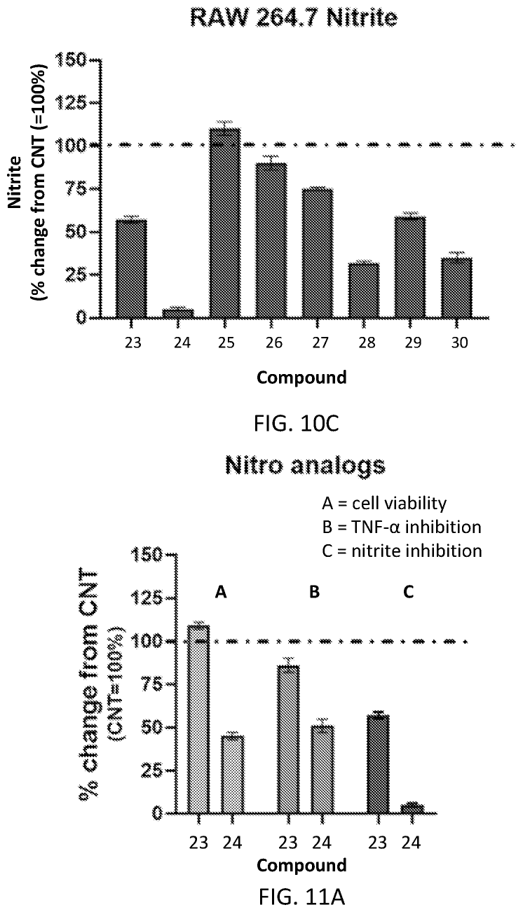

The compounds of Example 3 were screened against classical markers of inflammation using the LPS-activated RAW 264.7 cell model, a system readily adapted to identifying modulators of nitrite and TNF-α production (Tweedie et al., Open Biochem J 2011, 5:37-44). Ultimately 10 μM was selected as the concentration for further study, as this concentration provided adequate compound solubility concurrent with minimal cellular toxicity, all while furnishing notable effects on the markers of interest. The resulting inhibitory activities of the compounds on LPS-induced nitrite and TNF-α are summarized in Table 8 and FIGS. 10A-10C.

TABLE 8
Summary of Biological Data

| Compound | % Change versus Control (± s.e.m.) | | |
|---|---|---|---|
| (10 uM) | Viability | TNF-a | Nitrite |
| 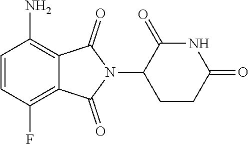 [SMILES: Nc1ccc(F)c2c1C(=O)N(C1CCC(=O)NC1=O)C2=O] | 104.9 ± 2.1 | 86.0 ± 4.4 | 56.8 ± 2.2 |
| 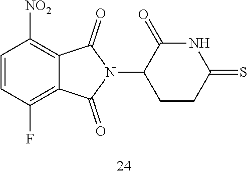 [SMILES: O=C1NC(=S)CCC1N1C(=O)c2c(F)ccc([N+](=O)[O-])c2C1=O] | 44.9 ± 1.9 | 51.3 ± 3.8 | 4.7 ± 1.0 |
| 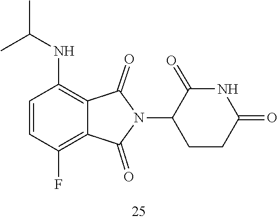 [SMILES: CC(C)Nc1ccc(F)c2c1C(=O)N(C1CCC(=O)NC1=O)C2=O] | 99.9 ± 0.4 | 87.1 ± 3.4 | 110.1 ± 4.4 |
| 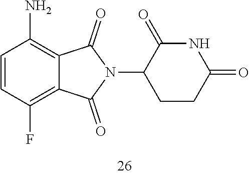 [SMILES: Nc1ccc(F)c2c1C(=O)N(C1CCC(=O)NC1=O)C2=O] | 100.9 ± 1.1 | 104.8 ± 4.6 | 90.3 ± 3.7 |
| 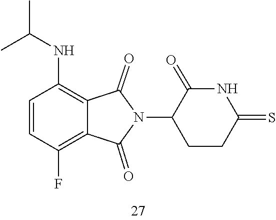 [SMILES: CC(C)Nc1ccc(F)c2c1C(=O)N(C1CCC(=S)NC1=O)C2=O] | 112.3 ± 1.7 | 94.4 ± 2.8 | 74.8 ± 1.2 |
| 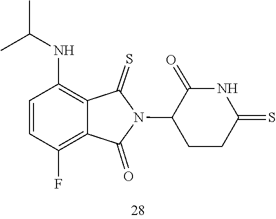 [SMILES: CC(C)Nc1ccc(F)c2c1C(=S)N(C1CCC(=S)NC1=O)C2=O] | 74.1 ± 4.2 | 74.7 ± 2.3 | 32.3 ± 1.1 |
| 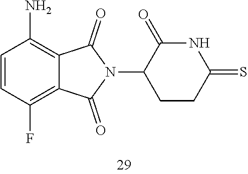 [SMILES: Nc1ccc(F)c2c1C(=O)N(C1CCC(=S)NC1=O)C2=O] | 112.4 ± 2.9 | 107.0 ± 4.0 | 59.0 ± 1.6 |
| 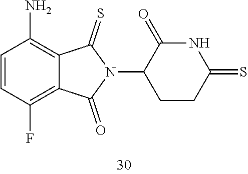 [SMILES: Nc1ccc(F)c2c1C(=S)N(C1CCC(=S)NC1=O)C2=O] | 96.1 ± 0.5 | 104.1 ± 2.5 | 35.3 ± 3.0 |

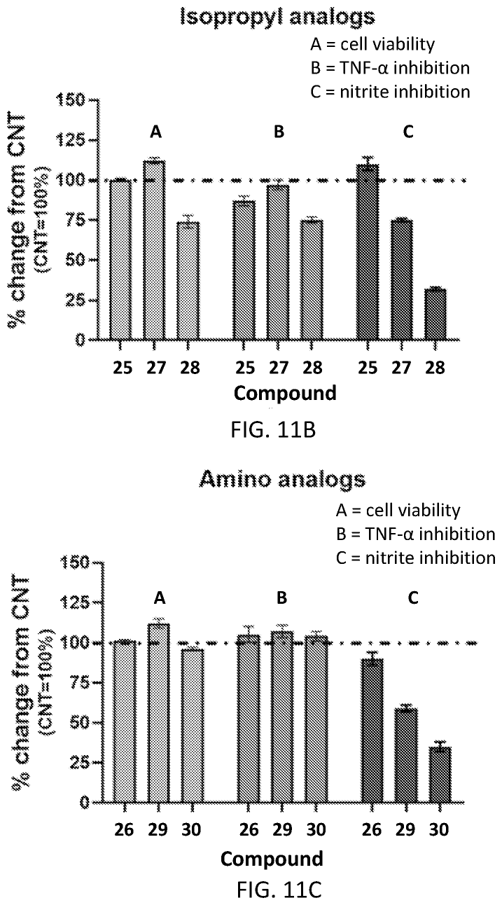

The compounds, in general, were very well tolerated with most retaining >80% cell viability at 10 μM treatment. In this regard, the most readily apparent outlier was monothionated analog 24. In fact, when the two nitrated analogs were directly compared (FIG. 11A), it was clear that thionation intensified cytotoxicity relative to the native oxo-material 23, while nitrate and TNF levels presumably diminished in accordance with associated cell death. Next, the N-isopropylated cohort revealed that increases in thionation effected corresponding decreases in nitrite levels concurrent with slight cytotoxicity (FIG. 11B). A comparable (albeit much weaker) overall trend was observed with TNF-α. Finally, when the primary amino analogs 26, 29, and 30 were compared, it was very clear that thionation led to reciprocal and significant decreases in nitrite while TNF-α levels were left unaffected (FIG. 11C). However, in contrast to the trends observed in the nitrated and isopropylated materials, each member of this group maintained >95% viability regardless of thionation level employed. This important distinction helped balance and clarify the differences in nitrite reduction observed between 28 and 30.

With the results of this preliminary screen in hand, a complementary study comparing supplementary candidates was performed. Pomalidomide compounds 3′ and 4′ were readily available from prior studies.

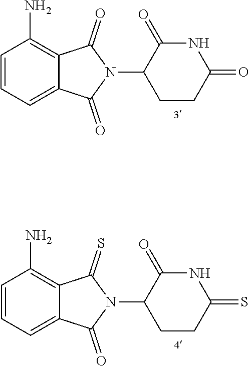 [SMILES: Nc1cccc2c1C(=O)N(C1CCC(=O)NC1=O)C2=O ; Nc1cccc2c1C(=S)N(C1CCC(=S)NC1=O)C2=O]

Attention was turned to compounds 31 and 32. The syntheses began with a simple condensation of 4-fluoro-3-nitrophthalic acid (US 2020/0061033 A1) with 3-aminoglutatrimide (Scheme 4, FIG. 8). The corresponding 5-fluoronitrothalidomde 34 was obtained in moderate yield. Subsequent reduction to 31 and dithionation yielded desired adduct 32.

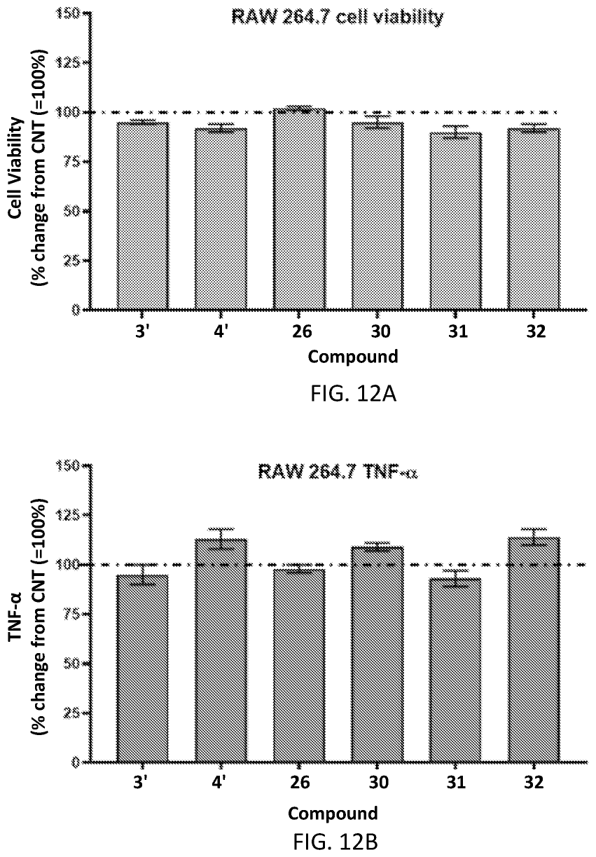

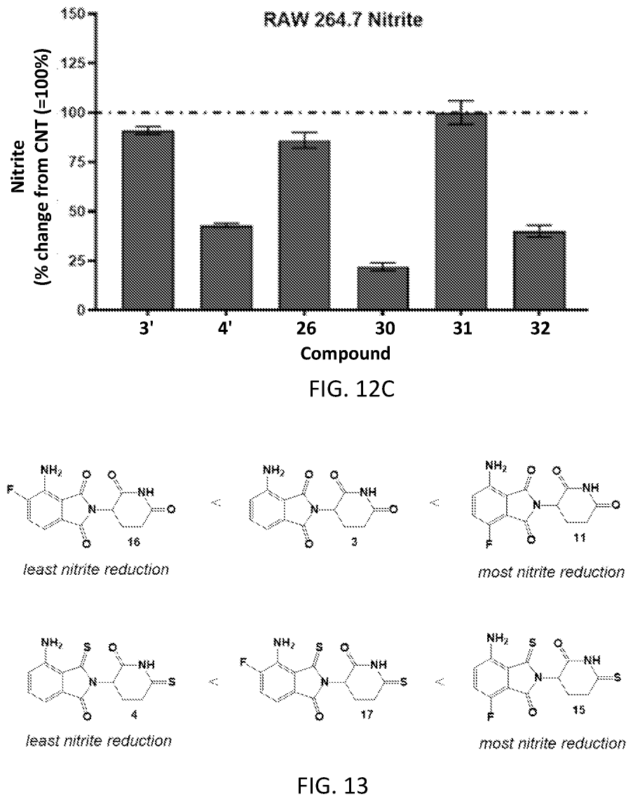

This concise six-compound grouping was collectively evaluated in a similar RAW cell assay as before, furnishing a direct, iterative appraisal of fluorine regiochemistry and carbonyl dithionation across a uniquely focused pomalidomide family (FIGS. 12A-12C and Table 9). As was expected, dithiopomalidomide 4′ reduced nitrite levels significantly relative to pomalidomide 3′. Interestingly, the newly prepared 5-fluorinated iterations found middle ground between the typical pomalidomides and their 7-fluoroinated isomers, as oxo-version 31 was surpassed by counterparts 3′ and 26, while 5-fluoro-dithiopomalidomide 32 demonstrated a modest efficacy sandwiched between that of dithiopomalidomide 4′ and the superior 7-fluoro-dithiopomaldiomide 30 (FIG. 13).

TABLE 9
Summary of Biological Data

| Compound | % Change versus Control (± s.e.m.) | | |
|---|---|---|---|
| (10 uM) | Viability | TNF-a | Nitrite |
| 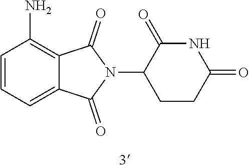 [SMILES: Nc1cccc2c1C(=O)N(C1CCC(=O)NC1=O)C2=O] | 95 ± 1.2 (N.S.) | 95 ± 4.8 (N.S.) | 91 ± 1.6 (N.S.) |
| 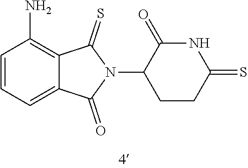 [SMILES: Nc1cccc2c1C(=S)N(C1CCC(=S)NC1=O)C2=O] | 92 ± 1.8 (N.S.) | 113 ± 5.4 (N.S.) | 43 ± 1.0 (N.S.) $ |
| 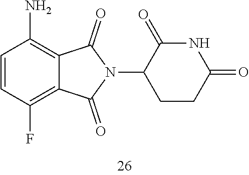 [SMILES: Nc1ccc(F)c2c1C(=O)N(C1CCC(=O)NC1=O)C2=O] | 102 ± 0.8 (N.S.) | 98 ± 1.5 (N.S.) | 86 ± 3.9 (N.S.) |
| 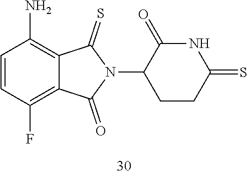 [SMILES: Nc1ccc(F)c2c1C(=S)N(C1CCC(=S)NC1=O)C2=O] | 95 ± 3.0 (N.S.) | 109 ± 2.4 (N.S.) | 22 ± 1.7 ( **** ) |
| 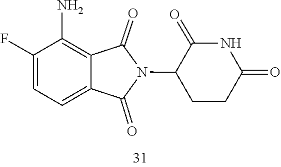 [SMILES: Nc1c(F)ccc2c1C(=O)N(C1CCC(=O)NC1=O)C2=O] | 90 ± 2.6 (N.S.) | 93 ± 4.1 (N.S.) | 100 ± 5.5 (N.S.) |
| 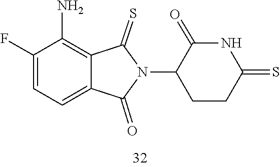 [SMILES: Nc1c(F)ccc2c1C(=S)N(C1CCC(=S)NC1=O)C2=O] | 92 ± 1.6 (N.S.) | 114 ± 3.9 (N.S.) | 40 ± 1.2 (N.S.) $ |

N.S. refers to not significant, **** refers to P < 0.0001 compared to appropriate vehicle + LPS control. Additionally, for values annotated with N.S. $, some data points were not normally distributed and required as Kruskal-Wallis test ANOVA with Dunn's multiple comparisons test. Using this analysis, these data points did not attain statistical significance.

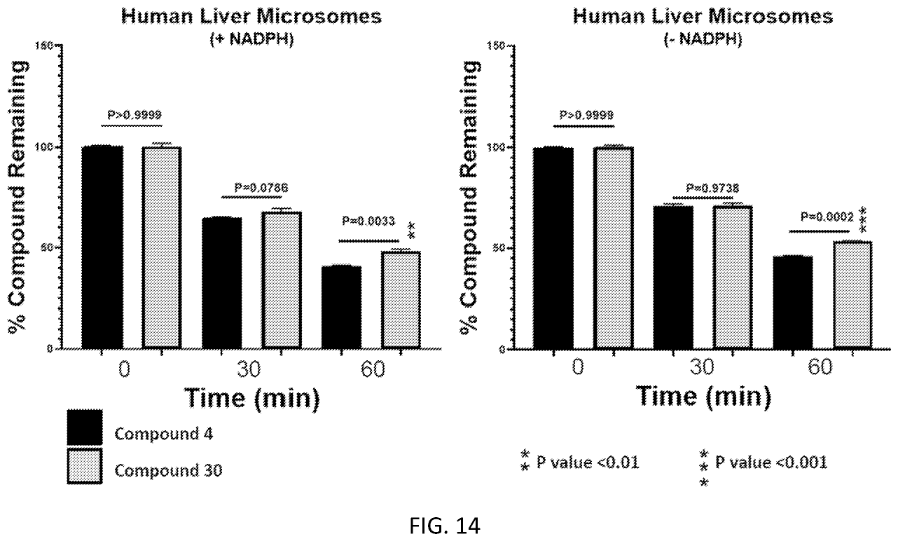

The fluorinated compounds were evaluated under the more metabolically demanding conditions of a Phase-1-type microsomal analysis. It was reasoned that at least one of the fluorinated analogs would demonstrate a measurable degree of stability over the primary non-fluorinated 3,6′-dithiopomaldomide 4′, especially when subjected to metabolically rigorous human liver homogenates. For this initial comparison, the 7-fluoro-dithiopomaldiomide 30 was selected owing to its combination of favorable biological parameters and general ease of synthesis. The reactions on each sample were initiated by addition of human liver microsomes in the presence or absence of an NADPH regenerating system, and compound disappearance was monitored via LC/MS (FIG. 14, Table 10).

TABLE 10
Phase I Metabolic Stability in Human Liver Microsomes

| | | % Compound | |
|---|---|---|---|
| | Time | Remaining (mean) | |
| Compound | (min) | NADPH | -NADPH |
| 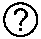 | 0 | 100 | 100 |
| | 30 | 64 | 71 |
| | 60 | 41 | 46 |
| 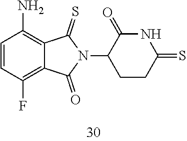 [SMILES: Nc1ccc(F)c2c1C(=S)N(C1CCC(=S)NC1=O)C2=O] | 0 | 100 | 100 |
| | 30 | 68 | 71 |
| | 60 | 48 | 53 |

As seen in FIG. 14 and Table 10, 3,6′-dithiopomaldomide 4′ demonstrated a moderate level of instability, both with and without NADPH fortification. After 1 hr incubation, more than half of the material had been consumed. In contrast, 7-fluoro-dithiopomaldiomide 30 demonstrated heightened resistance under the same conditions, yielding 7% more stability in the absolute sense, equating to roughly 16% more material remaining when compared to its non-fluorinated partner. In all cases, the materials seemed to be more sensitive to +NADPH processes versus those lacking NAPDH.

Discussion: A focused family of aminofluorophthalimides and selected thionated analogs was synthesized. Through careful examination of the associated cell viability and anti-inflammatory data, preliminary structure activity relationships were established. In general, the materials were well tolerated, with only compound 24 displaying considerable cellular toxicity at 10 μM. While anti-TNF-α activities were marginal across all compounds, nitrite levels were clearly affected by both fluorination and thionation. Site-specific fluorination and thioamidation provided compound 30, which when dosed at 10 μM in stimulated RAW cells, effected an 65% drop in LPS-induced nitrite production concurrent with minimal perturbations to TNF-α and cell viability. Furthermore, during the course of human liver microsomal analyses, compound 30 demonstrated enhanced metabolic fortitude relative to its non-fluorinated counterpart 4′, a result in line with an increased stability afforded by a site-specific 7-fluorination at the metabolically-sensitive pomalidomide locus.

### Example 5

### Anti-Inflammatory Activity in Mitigating Traumatic Brain Injury (TBI) in a Mouse-Controlled Cortical Impact Model

TBI neuropathology is typically described as the result of two major biochemical phases. The first is represented by the actual primary injury that results from an external mechanical force applied to the head, and is characterized by tissue damage, vascular injuries (including interruption of the blood-brain barrier) and diffuse axonal injury. Depending on the nature of the trauma, skull fracture may be present. These mechanisms largely result in necrotic cell death, which can be more or less substantial depending on the severity of the injury (LaPlaca et al., J Neurotrauma 2007, 18:369-376; Frattalone and Ling. Neurosurg Clin N Am. 2013, 24, 309-19; Greig et al., Alzheimer Dement. 2014; 10 (S1): S62-S75). The long-lasting secondary phase of TBI consists of a series of biochemical cascades triggered by the primary trauma, and includes neuroinflammation, ischemia, mitochondrial dysfunction, glutamate excitotoxicity and hypoxia. These mechanisms ultimately result in synaptic dysfunction and apoptotic neuronal loss (Frattalone and Ling. Neurosurg Clin N Am. 2013, 24, 309-19; McKee and Daneshvar, Handbook Clin Neurol. 2015127:45-66; Ghajar, Lancet 2000, 356 (9233): 923-9; Morganti-Kossmann et al., Injury 2007, 38:1392-400). Whereas the primary phase, consequent to its immediate nature, is not easily treatable, the biochemical mechanisms involved in the secondary stage of TBI may represent potentially druggable targets (Diaz-Arrastia et al., J Neurotrauma 2014, 31:135-58).

Neuroinflammation, in particular, has been demonstrated to play a major role in the pathophysiology of post-TBI pathologies. Whereas an acute neuroinflammatory response is beneficial in instigating a reparative process after the injury, its excess and/or chronicity promotes a toxic cellular microenvironment that will ultimately translate into neuronal dysfunction and death (Morganti-Kossmann et al., Curr Opinion Crit Care 2002, 8:101-105; Frankola et al., CNS Neurol Disord Drug Targets 2011, 10:391-403; DiSabato et al., J Neurochem 2016, 139 (Suppl 2): 136-152; Simon et al., Nat Rev Neurol. 2017, 13:572). Simplistically, following a TBI insult microglial cells begin mediating an immune response to the insult by switching from a quiescent (M2) to an activated (M1) phenotype, which stimulates a series of pro-inflammatory mechanisms (Kumar et al., J Neurotrauma 2016, 33:1732-50; Simon et al., Nat Rev Neurol. 2017, 13:572; Donat et al., Front Aging Neurosci. 2017, 9:208). Markers of activated microglial cells have been shown to be elevated shortly after TBI in preclinical models (Chiu et al., J Neurosci Methods 2016, 272:38-49; Lin et al., eLife 2020, 9: e54726; Readnower et al., J Neurotrauma 2011, 28:1845-53) and human studies (Lier et al., Int J Legal Med. 2020, 134:2187-93; Johnson et al., Brain 2013, 136 (Part1): 28-42; Ramlackhansingh et al., Ann Neurol. 2011, 70:374-78). One of the main pro-inflammatory mediators whose levels are increased by activated glial cells is tumor necrosis factor (TNF)-α. Animal and clinical studies have demonstrated an elevated presence of this cytokine following TBI (Frugier et al., J Neurotrauma 2010, 27:497-507; Dalgard et al., Front Mol Neurosci. 2012, 5:6).

Compounds 14 (tetrafluorobornylphthalimide, TFBP) and 16 (tetrafluoronorbornylphthalimide, TFNBP) were evaluated in a controlled cortical impact (CCI) mouse model of TBI.

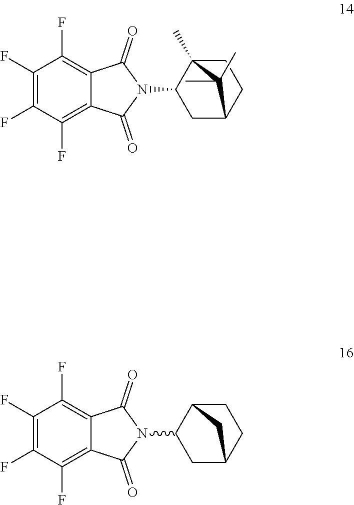 [SMILES: CC1(C)[C@@H]2CC[C@]1(C)[C@@H](N1C(=O)c3c(F)c(F)c(F)c(F)c3C1=O)C2 ; O=C1c2c(F)c(F)c(F)c(F)c2C(=O)N1C1C[C@H]2CC[C@@H]1C2]

These compounds retain their phthalimide core structure of thalidomide and its clinical analogs, but fluorination blocks potential for phase 1 metabolic process around the bicyclic ring. Furthermore, the planar glutarimide ring structure of thalidomide and clinical analogs is replaced by a bridged ring, space occupying caged-like structure designed to sterically hinder potential binding with cereblon. Cereblon is a key target of thalidomide and its alike analogs, and forms a critical component of E3 ubiquitin ligase that marks the critical transcription factors SALL4, Ikaros and Aiolos, for degradation (Chamberlain et al., Drug Discov Today Technol 2019, 31:29-34). The ubiquitination of these proteins account for the antineoplastic but also the teratogenicity of currently available immunomodulatory imide drugs (IMiDs). As shown in detail below, studies of compound 14 demonstrated its ability to lower levels of LPS-induced TNF-α in RAW 264.7 cell cultures. The anti-inflammatory potential of this compound was then confirmed with in vivo studies, in an LPS model of both systemic and CNS inflammation as well as in a controlled cortical impact model (CCI) of moderate TBI.

### Methods

RAW 264.7 cell culture: RAW 264.7 mouse cells were purchased from ATCC (Manassas, VA, USA) and grown in DMEM media supplemented with 10% FCS, penicillin 100 U/ml and streptomycin 100 μg/ml (ThermoFisher Scientific, Asheville, NC, USA). The cells were maintained at 37° C. and 5% CO2. Cells were propagated as described by ATCC guidelines. RAW 264.7 cells were cultured following the methods described in Tweedie et al. (Open Biochem J 2011, 5:37-44). Cells were treated with drug vehicle (DMSO, Sigma, St Louis, MO, USA) or the test compounds (WL-V-157AG-1 (157AG) or WL-V-158G-1, 100 nM-1 μM), with 3-4 wells per treatment group (n=3-4). One hour after the addition of the vehicle/test compound the cells were challenged with LPS (30-60 ng/ml, E. coli 055: B5, Sigma, St Louis, MO, USA). Twenty-four hours later the media was harvested and analyzed to assess cell viability and for the quantification of secreted TNF-α protein (ELISA MAX™ Deluxe Set Mouse TNF-α, BioLegend, CA, USA) and nitrite (Griess Reagent System, Promega, Madison, WI, USA). Cellular health was assessed by use of the CellTiter 96 AQueous One Solution Cell Proliferation Assay (Promega, Madison, WI, USA).

### Cereblon Binding and Neosubstrate Ubiquitination:

A bead-based AlphaScreen technology was adopted for cereblon binding in Human SH-SY5Y Neuronal Cultures with minimal modifications from the manufacturer's protocol (BPS Bioscience catalog no. 79770). ‘Test compounds’ or pomalidomide (as a positive control) were incubated with reaction mixtures including cereblon/DNA damage-binding protein 1-Cullin 4a-ring-box protein 1 complex (CRBN/DDB1-CUL4A-Rbx1, 12.5 ng) and bromodomain-containing protein 3 (BRD3) (6.25 ng) in an Optiplate 384-well plate (PerkinElmer catalog no. 6007290). After 30 min of incubation with shaking at room temperature, AlphaLISA anti-FLAG Acceptor and Alpha Glutathione Donor beads (PerkinElmer) were sequentially added and then incubated for 1 hr at room temperature for each of the added chemicals. Alpha counts were thereafter read on a Synergy Neo2 (BioTek) for the analysis. Relative activity or inhibition was calculated as the highest value, which was set to 100%, and the lowest value was set to 0% after subtraction of the “blank value” from all readings.

The activity of ‘test compounds’ (or pomalidomide—as a positive control) on neosubstrates was evaluated in MM1.S (myeloma) and H9 hESC (human embryonic stem) cell lines. Specifically. MMLS cells were obtained from ATCC (Manassas, VA), grown in RPMI media supplemented with 10% FBS, penicillin 100 U/mL, and streptomycin 100 mg/mL, and were maintained at 37° C. and 5% CO2. MM1.S cells were treated with 10 μM of test compounds (together with thalidomide, lenalidomide and/or pomalidomide as a positive control) for 24 hr; thereafter, their cell lysates were prepared for Western blot analysis, as described previously (Lecca et al., Alzheimers Dement. 2022 Mar. 2. doi: 10.1002/alz.12610. Online ahead of print). In contrast, H9 hESC lines were obtained from WiCell Research Institute (catalog no. WA09; Madison, WI) and grown on growth factor reduced matrigelcoated dishes in mTeSR1 media (STEMCELL Technologies, Vancouver, Canada), supplemented with 5 ng/mL bFGF, penicillin 100 U/mL, and streptomycin 100 μg/mL and maintained at 37° C. and 5% CO2. H9 hESC cells were treated with 20 μM of thalidomide analogs (thalidomide, pomalidomide, and F-3,6′-DP) for 24 hr, and their cell lysates prepared for the Western blot analysis, as described previously [Tsai et al., Pharmaceutics 2022; 14:950].

For Western blot analysis, total proteins were extracted using RIPA buffer (ThermoFisher Scientific, Waltham, MA) containing Halt Protease Inhibitor Cocktail (ThermoFisher Scientific). Thereafter, proteins were separated by gel electrophoresis and then transferred to polyvinylidene difluoride (PVDF) membranes (ThermFisher Scientific), as described previously [Lin et al., eLife 2020; 9: e54726; Tsai et al., Pharmaceutics 2022; 14:950]. The following primary antibodies were used: (i) anti-Ikaros antibody (CST catalog no. 9034; 1:1000; Cell Signaling Technology, Danvers, MA), (ii) anti-Aiolos antibody (CST catalog no. 15103; 1:1000; Cell Signaling Technology), (iii) anti-SALL4 antibody (SC101147; 1:1000; Santa Cruz Biotechnology, Dallas, TX), and (iv) anti-GAPDH antibody (CST catalog no. ab8245; 1:5000; Abcam). After incubation at 4° C. overnight, the following HRP conjugated secondary antibodies were used: (i) goat anti-rabbit IgG (ThermoFisher Scientific) for ikaros and aiolos, and (ii) goat anti-mouse IgG (ThermoFisher Scientific) for SALL4 and GAPDH. GAPDH, a protein that is generally expressed in all eukaryotic cells, was used as an internal control against which the other protein expression levels were compared. Antigen-antibody complexes were detected using enhanced chemiluminescence (Thermo, iBright CL1500).

Animal studies: All rodents were housed at 25° C. in a 12/12 hr light/dark cycle with access to food and water ad libitum. All efforts were made to minimize any potential animal suffering and as well as the number of animals used. The procedures used in this study were fully approved by following the Institutional Animal Care and Use Committees (Intramural Research Program, National Institute on Aging, NIH (animal protocols 331-TGB-2024; 488-TGB-2022). All efforts were undertaken to minimize any potential animal suffering and as well as the number of animals used by incorporating the outcome measures from our prior studies (Hsueh et al., Cell Transplant 2019, 28:1183-96) and a power analysis (Charan et al., J Pharmacol Pharmacother 2013, 4:303-6).

LPS model of inflammation in rats: Male Fischer 344 rats (Charles River Laboratories, Wilmington, MA, USA) (approx. 150 g weight) were randomly assigned across groups, and thereafter administered either vehicle (Veh), or TFBP or TFNBP. Two doses of TFBPs were evaluated: (TFBP: 16.25 mg/kg or 32.5 mg/kg, and for TFNBP: 14.33 mg/kg or 28.66 mg/kg), i.p. These compounds were suspended in 1% carboxymethyl cellulose (CMC) in saline (0.9%), and administered 60 min prior to either administration of LPS (1 mg/kg, Sigma, St Louis, MO, E. coli O55:B5 in saline (0.9%), 0.1 ml/kg i.p.) or of Vehicle (CMC in saline (0.9%) 0.1 ml/kg, i.p.). The selected drug doses are equimolar to that of pomalidomide (12.5 mg/kg and 25 mg/kg), which have been demonstrated to be well-tolerated in prior rodent studies, and are of translational relevance to humans (Tweedie et al., J Neuroinflamm. 2012, 9:106; Baratz et al., J Neuroinflamm. 2015, 12:45). At 4 hr following LPS or Veh, animals were euthanized, and plasma and brain (hippocampus) tissue samples were collected and stored at −80° C. Brain samples were later sonicated in a Tris-based lysis buffer (Mesoscale Discovery, Gaithersburg, MD, USA) with protease/phosphatase inhibitors (Halt™ Protease and Phosphatase Inhibitor Cocktail, ThermoFisher Scientific, Asheville, NC, USA, diluted to 3×). Next, brain samples were centrifuged (10,000 g, 10 min, 4° C.), and protein concentrations were determined by Bicinchoninic acid assay (BCA, ThermoFisher Scientific, Asheville, NC, USA). An ELISA for TNF-α, IL-6, IL-1B, IL-10 and IL-13 was later performed following the manufacturer's protocol (Mesoscale Discovery).

In vivo model of TBI: Mice were anesthetized with 2.5% tribromoethanol (Avertin: 250 mg/kg; Sigma, St. Louis, MO, USA) and placed in a stereotaxic frame (Kopf Instruments, Tujunga, CA, USA). A 4 mm craniotomy was performed using sterile procedures; the craniotomy point was selected midway between the lambda and bregma sutures and laterally midway between the central suture and the temporalis muscle. The skull fragment was then carefully removed without disruption of the underlying dura. Prior to injury induction, the tip of the impactor was angled and kept perpendicular to the exposed cortical surface. The mouse CCI instrument consisted of an electromagnetic impactor, Impact One (Leica Biosystems Inc., Buffalo Grove, IL, USA) that allows independent alteration of injury severity by controlling contact velocity and the level of cortical deformation. In these experiments, the contact velocity was set at 5.0 m/sec, the dwell time was set at 0.2 s and the deformation depth was set at 2 mm to produce a moderate TBI. The injury site was allowed to dry prior to suturing the wound. During surgery and recovery, the body temperature of the animals was maintained at 36-37° C. by using a heating pad. Based on data generated from prior LPS experiments in rats where TFBP demonstrated greater efficacy compared to its close analog TFNBP, TFBP was chosen for further investigations in the CCI model. Mice were randomized following CCI to treatment with either of two doses of TFBP (16.25 mg/kg and 32.5 mg/kg, i.p.) or saline, and were dosed at 1 and 24 hr after the injury.

Behavioral assessment for motor functions and coordination: Behavioral tests were performed 1 week and 2 weeks after injury, to assess changes in motor functions and coordination. All tests were performed during the animals' light phase; cages were transported to testing rooms at least 30 min prior to testing.

Beam Walking Test (BWT): A BWT was used to assess CCI-induced deficits in fine motor coordination. Mice have a preference for a darkened enclosed environment, as compared to an open illuminated one. Each animal was placed in darkened goal box for a 2 min habituation and then the trial began from the other (light) end of the beam. The beam was constructed with the following dimensions: 1.2 cm (width)×91 cm (length). The time taken for each animal to traverse the beam to reach the dark goal box and the immobility time spent between the moment when they were initially placed in the beam and when they started walking were documented. Five trials were recorded for each animal before CCI and at 1 and 2 weeks after CCI. The mean times to traverse the beam and the immobility times were calculated, and a plot was generated to evaluate treatment effects; these times were then used for statistical analysis.

Gait analysis: For the gait analysis, mice were tested on a fixed-speed treadmill apparatus (DigiGait; Mouse Specifics). Mice were habituated to the apparatus for 1 min, and then given a 1-min run at 5 cm/s. Following a 1-min rest, the treadmill speed was increased to 15 cm/s. Video was collected at high speed from a ventrally placed camera, and 3-5 s of representative gait video was selected by an experienced but blinded user for automated analysis.

Tissue processing: Two weeks after injury, mice were deeply anesthetized with isoflurane and perfused transcardially with 30 ml phosphate buffered saline (PBS). After removal, the brain was post-fixed with 4% PFA overnight then transferred to a 30% sucrose solution in PBS. Coronal sections from the dorsal hippocampus and the posterior parietal cortex were cryosectioned at 25 μm thickness and stored in cryoprotectant solution for Giemsa staining and immunohistochemical analysis.

Quantification of brain lesion and lateral ventricle size in TBI animals: One set of brain sections 2-weeks post-CCI were mounted on slides. The slices were then stained with 10% Giemsa KH₂PO₄ buffered solution (pH 4.5) for 30 min at 40° C. After a brief rinse, slides were destained, differentiated, and dehydrated in absolute ethanol. Thereafter, the sections were cleared in xylene and then coverslipped. Brain image regions were quantified using ImageJ 1.52q software (National Institutes of Health, Bethesda, MD). The calculation formula for contusion volume size and lateral ventricle size were as follows: Σ (area of contralateral hemisphere−area of ipsilateral hemisphere)/Σ(area of contralateral hemisphere); Σ(area of ipsilateral lateral ventricle)/Σ(area of contralateral lateral ventricle). There were 9 brain sections from each mouse counted by blinded observers, with regions starting from bregma at 0.86 to −1.46 mm.

Immunofluorescence analysis: For GFAP and Iba1 quantification, brain samples were incubated overnight with either one of the following primary antibodies: anti-GFAP 1:2500 dilution (chicken polyclonal, Abcam, USA, cat #ab4674) or anti-Iba1 1:200 dilution (guinea pig polyclonal anti-Iba1, Synaptic System, USA, cat #234004). After PBS washing, sections were incubated for 1 hr at RT with a goat anti-chicken AlexaFluor® 488-conjugated secondary antibody IgY (H+L) 1:500 dilution (ThermoFisher, USA, cat #A-21432) for detection of GFAP, and with a Goat anti-Guinea Pig IgG (H+L) Highly Cross-Adsorbed Secondary Antibody, Alexa Fluor™ 555, ThermoFisher, USA, cat #A-21435) for detection of Iba1. Sections were washed in PBS and mounted with VECTASHIELD® Antifade Mounting Medium with DAPI (Vectorlabs, USA, cat #H-1200). Controls consisted of omission of the primary antibody. Iba1 morphological analysis was performed on ×40 magnification images by using MotiQ, a plugin for Image J. MotiQ thresholder (v0.1.2) was used to create figures from immunofluorescences for the MotiQ analyser (v0.1.3). Multiple parameters were analyzed, including ramification index, spanned area, number of branches, junctions and endpoints.

Statistical analysis: Data were evaluated between groups with one-way analysis of variance (ANOVA) followed by Dunnett's posthoc tests (GraphPad Prizm 7, San Diego, CA, USA) when appropriate for multiple comparisons. Bar graphs are presented as mean±SEM values. A p value of <0.05 was considered significant.

### Results

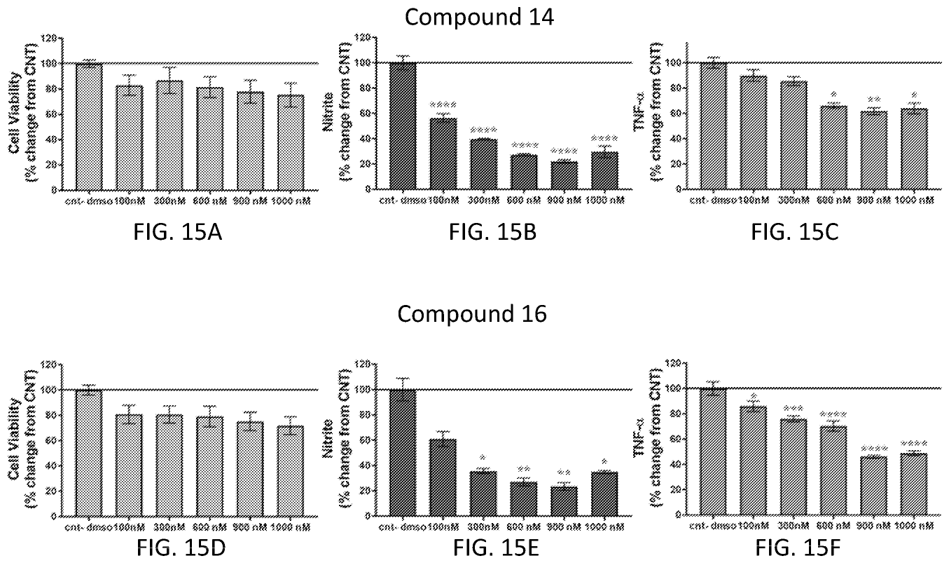

Compounds 14 and 16 were well tolerated in cellular studies and mitigated elevated nitrite and TNF-α levels in RAW 264.7 mouse cells challenged with LPS. Challenge with LPS induced a spike in levels of nitrite and TNF-α, as compared to non-treated control cells (not shown). Treatment of RAW 264.7 macrophage-like cell cultures with compound 14 or compound 16 significantly decreased levels of LPS-induced nitrite and TNF-α in the cell culture media, without negatively impacting cell viability (FIGS. 15A-15F). Specifically, nitrite levels were significantly reduced starting at 100 nM concentration for compound 14 and 300 nM for compound 16, as compared to the LPS+ Vehicle group (FIGS. 15B, 15E). Compound 14's action was less pronounced on TNF-α expression, and it induced significant TNF-α declines at concentrations equal to 600 nM or more (FIG. 15C). For compound 16 this was achieved at 300 nM and above (FIG. 15F). Cell viability was not significantly affected by either agent across the concentrations evaluated (100-1000 nM), as compared to the LPS+Vehicle group, (FIGS. 15A, 15D).

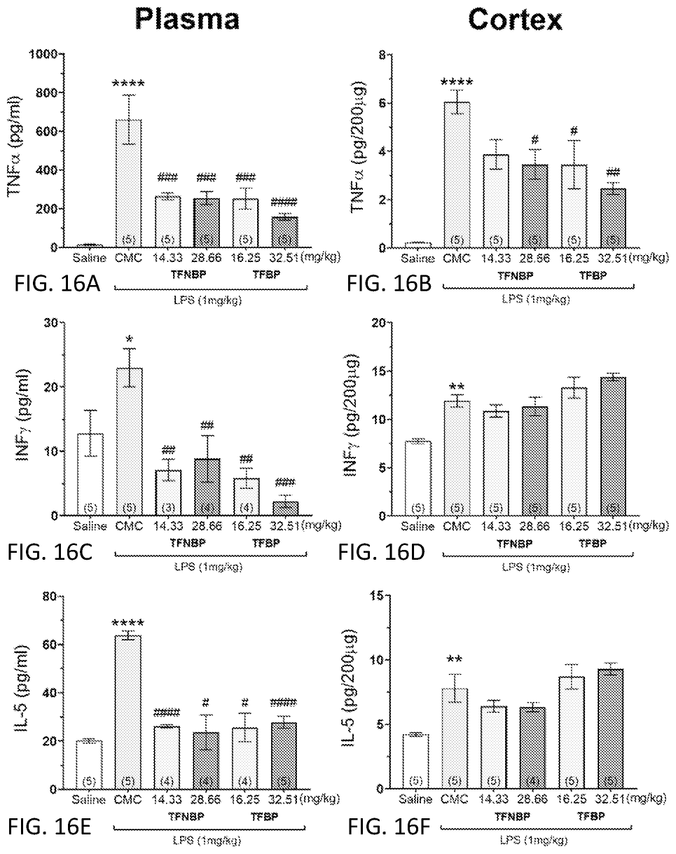

Compounds 14 and 16 significantly decreased levels of TNF-α, IFN-γ and IL-5 in plasma and cortex of LPS-challenged animals. As a first in vivo approach, the ability of both compound 14 and compound 16 to reduce LPS-mediated increases of proinflammatory cytokines in rats was evaluated. The results are shown in FIGS. 16A-16F, where compound 14 is labeled TFBP and compound 16 is labeled TFNBP. Consistent with a previous study from our group (Tweedie et al., J Neuroinflammation 2012; 9:106), the systemic administration of LPS (1 mg/kg, i.p.) resulted in a substantial and statistically significant increment in TNF-α plasma levels, which were elevated from 14.3±6.4 to 661.2±126.7 pg/ml, a 46-fold elevation at 4 hr post LPS challenge (FIG. 16A), a time previously demonstrated to provide an approximate steady-state for TNF-α generation in response to LPS (Tweedie et al., J Neuroinflammation 2012; 9:106). Both evaluated doses of compound 14 and compound 16 reduced this increase (p<0.001), with the higher dose of compound 14 showing the greatest efficacy (decline: −76% vs. LPS alone group, p<0.0001). Cerebral cortex levels of TNF-α were likewise elevated in LPS-challenged animals, as compared to the control group without LPS (from 0.2±0.02 to 6±0.5 pg/200 μg, p<0.0001, a 30-fold elevation. Treatment with compound 14 countered this increase more potently than compound 16. Specifically, both low and high doses of compound 14 were able to mitigate this rise (−41.7% and −58.3%, respectively), whereas compound 16 only effectively did so at the higher tested dose (−41.7% vs. LPS group, FIG. 16B). Systemic LPS administration, additionally, elevated levels of IFN-γ in cerebral cortex, from 7.74±0.25 pg/ml to 11.92±0.63 pg/ml, as well as plasma (p=0.053); treatment with compound 16 and, more potently, with compound 14 proved able to reduce this rise (p<0.01 vs. control group for TFNBP low and high dose, as well as compound 14 low dose; p<0.001 for compound 14 high dose vs. control). Brain cortex IFN-γ levels were not impacted by either compound (FIGS. 16C, 16D). Cerebral cortex and plasma levels of IL-5 were elevated following LPS challenge (p<0.01 and p<0.0001 respectively, vs control group). Treatment with compounds 14 and 16 at both doses reduced IL-5 expression in plasma (#p<0.05 and ####p<0.0001 vs control group, but not brain. Other inflammatory cytokines were quantified, but no statistically significant changes among experimental groups were detected (data not shown).

On the basis of the more effective mitigation of LPS-induced systemic and neuroinflammation provided by compound 14 in comparison to equimolar compound 16, the former was selected for evaluation of efficacy to counter a TBI challenge in rodents subjected to CCI. This TBI model has a well characterized neuroinflammatory component. Furthermore, it is associated with an early motor impairment that is evident at one-week post-injury that gradually resolves over a subsequent week (Hsueh et al., ACS Phamacol Transl Sci. 2021; 4:980-1000; Hsueh et al., Neurobiol Dis. 2019; 130:104528).

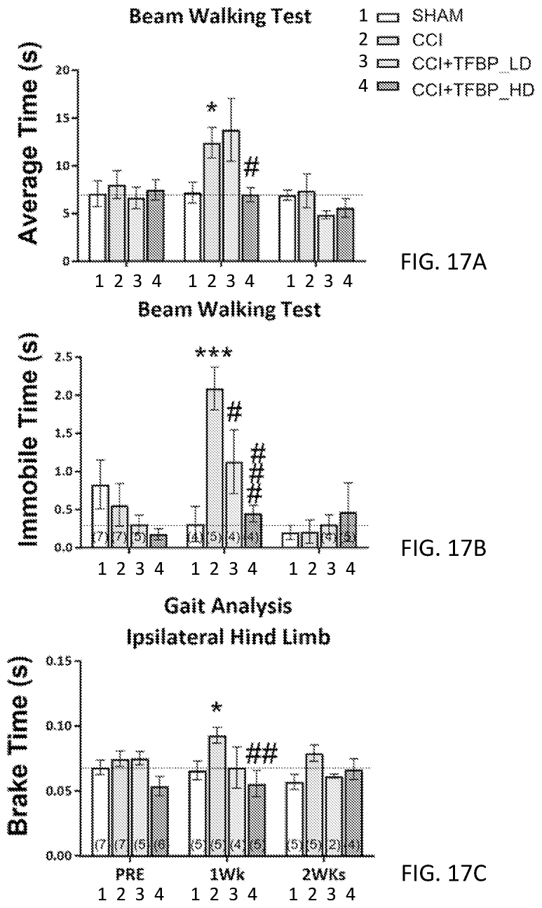

Compound 14 significantly mitigated TBI-induced motor function deficits in mice. To evaluate the ability of compound 14 to mitigate motor deficits induced by CCI injury, behavioral tests were performed at 1 week and 2 weeks after TBI. The BWT was used as an assessment of motor coordination for TBI-challenged animals, by measuring (a) the average time that animals took to walk the platform, and (b) an immobility time that they spent at the platform starting point before beginning to walk. The CCI-alone (Vehicle) group exhibited a rise in both average transit time (*p<0.05 vs. sham group (without CCI)) and immobility time (***p<0.001 vs. sham group). Compound 14 at the higher tested dose prevented the CCI-induced behavioral impairment, as assessed by both measures (#p<0.05 for average transit time; ###p<0.001 for immobility time). Treatment with the lower TFBP dose proved effective in mitigating the immobility measure (#p<0.05 vs. CCI-alone group), but not the average transit time deficit. As noted in a prior similar CCI study (Hsueh et al., ACS Phamacol Transl Sci. 2021; 4:980-1000), deficits in BWT largely were resolved at two-weeks, and hence no statistically significant differences were observed across all groups at two weeks after CCI (FIGS. 17A, 17B).

The Gait analysis test was used to assess potential changes in spontaneous locomotion. Among the parameters analyzed by the DigiGait software, we observed that CCI-alone animals at one week showed an increased brake time, which represents the duration between the initial and the maximum paw contact, starting after the swing phase (*p<0.05 vs sham group). Treatment with the high dose of compound 14 fully mitigates this rise (##p<0.001). No statistically significant differences were observed across groups at two weeks after CCI (FIG. 17C).

As compound 14 demonstrated mitigation of motor deficits at 1 week post CCI and functional deficits were not evident across groups at 2 weeks post CCI, we interpret this as compound 14 speeding spontaneous recovery of motor coordination following a TBI. To evaluate how this was achieved, immunohistochemical studies were subsequently undertaken to quantify the actions of compound 14 vs. Vehicle in relation to the TBI-induced lesion area and subsequent development of microglial mediated neuroinflammation.

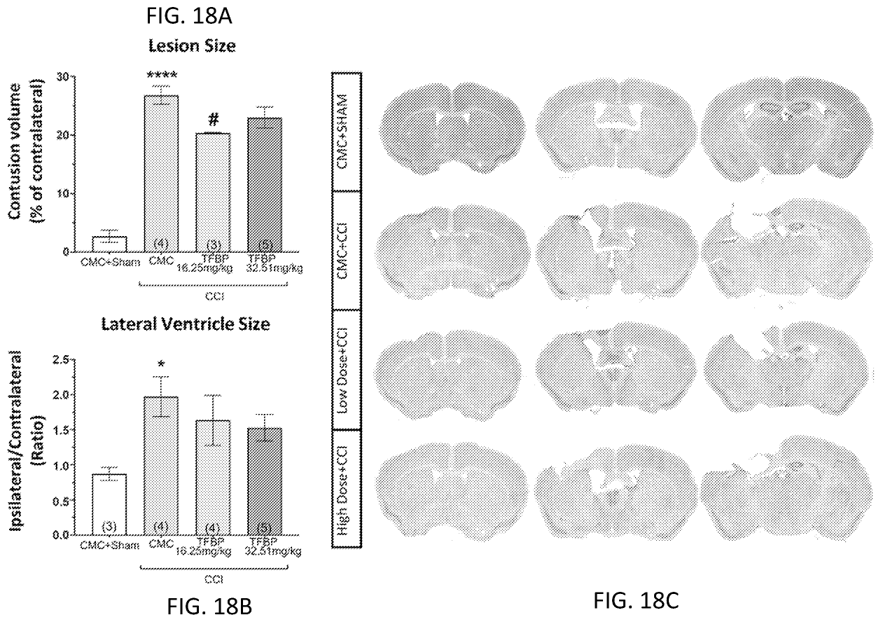

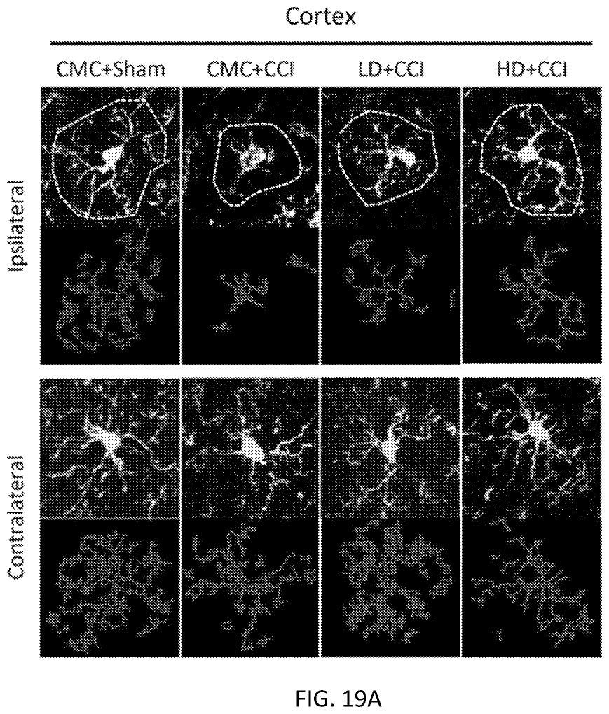

Compound 14 significantly decreased cortical lesion volume in CCI-challenged mice. Giemsa histological staining was performed at two weeks after TBI to evaluate the contusion size and the lateral ventricle enlargement, which provides an indication of changes in intracranial cerebrospinal fluid (CSF). A direct result of the CCI procedure was a loss of cortical tissue around the TBI site, expressed as a percentage of the contralateral hemisphere (****p<0.0001 vs sham). Treatment with the compound 14 lower dose proved able to reduce loss of cortical tissue in CCI-challenged mice (*p<0.05 vs CCI). A trend to decline was evident in the higher compound 14 dose group that failed to reach statistical significance. An expansion in the size of the lateral ventricle was evident on the side ipsilateral to CCI injury in the CCI alone group (*p<0.05 vs. sham group (without CCI)). A decreasing trend was noticeable in the compound 14 treated groups in relation to this measure, but did not reach statistical significance (FIGS. 18A, 18B). FIG. 18C shows representative images of Giemsa-stained cortical sections.

Compound 14 mitigates TBI-mediated expression of activated microglial cells. As neuroinflammation is a hallmark of CCI-induced TBI (Chiu et al., J Neurosci Methods 2016, 272:38-49; Lin et al., eLife 2020, 9: e54726; Readnower et al., J Neurotrauma 2011, 28:1845-53) and is associated with a change in microglial phenotype, MotiQ analysis with ImageJ software allowed us to evaluate multiple morphological parameters indicative of different microglial quiescent vs. activated phenotypes. Under physiological conditions, microglia adopt a ‘resting’ phenotype, characterized by a small soma and long and thin processes. In response to an insult, such as a CCI procedure, microglial cells begin to mediate an immune response by switching to an ‘activated’ proinflammatory state, morphologically characterized by an amoeboid shape, with an enlarged soma as well as thicker and shorter processes.

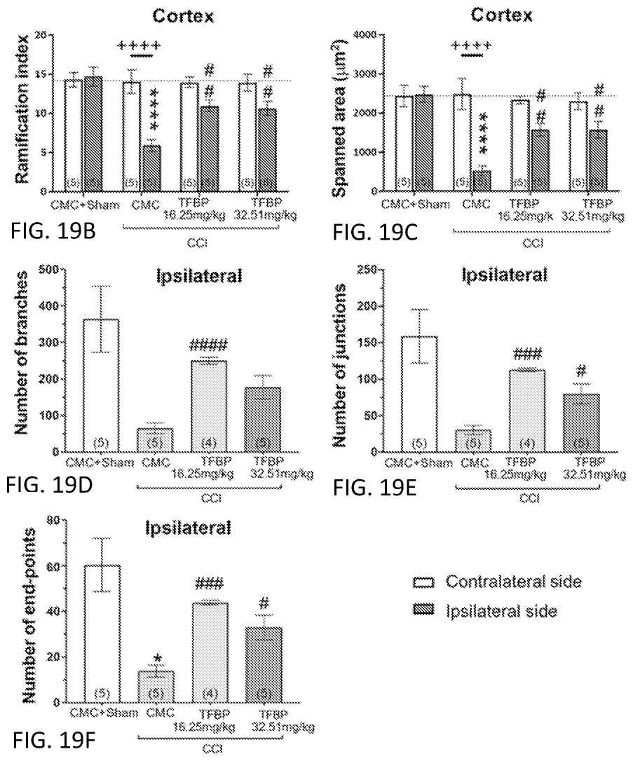

FIG. 19A shows representative images of Iba1+ cells at 40× magnification and their skeleton reconstructions through MotiQ software. In comparison to sham control mice, microglia present in the cortical region ipsilateral to injury in the CCI-alone group expressed dramatic reductions in these key morphological features characteristic of a resting (quiescent) phenotype, including ramification index (FIG. 19B, ****p<0.0001 vs. sham group), spanned area in μm² (FIG. 19C, ****p<0.0001 vs. sham group), number of branches (FIG. 19D), number of junctions (FIG. 19E), and number of end points (FIG. 19F, *p<0.05 vs. sham group). Notably, the contralateral side to the CCI lesion showed no statistically significant changes across groups in relation to these same morphological features (FIGS. 19B and 19C; data not shown for the other parameters). Treatment with both the lower and higher dose compound 14 substantially counteracted these CCI-induced microglial phenotypic measures on the ipsilateral side, including ramification index and spanned area (##p<0.001 vs. sham group), as well as the number of end points (###p<0.001 and #p<0.05 for the low and high doses tested, respectively, vs. sham group). A statistically significant reduction after treatment compared to the CCI group was also observed in relation to the number of branches (####p<0.0001 for compound 14 low dose group, vs. CCI) and junctions (###p<0.001 for the low dose; #p<0.05 for the high dose, vs CCI).

TFBP (compound 14) and TFNBP (compound 16) cereblon interactions: TFBP and TFNBP did not bind cereblon and did not lower the expression of neosubstrate SALL4.

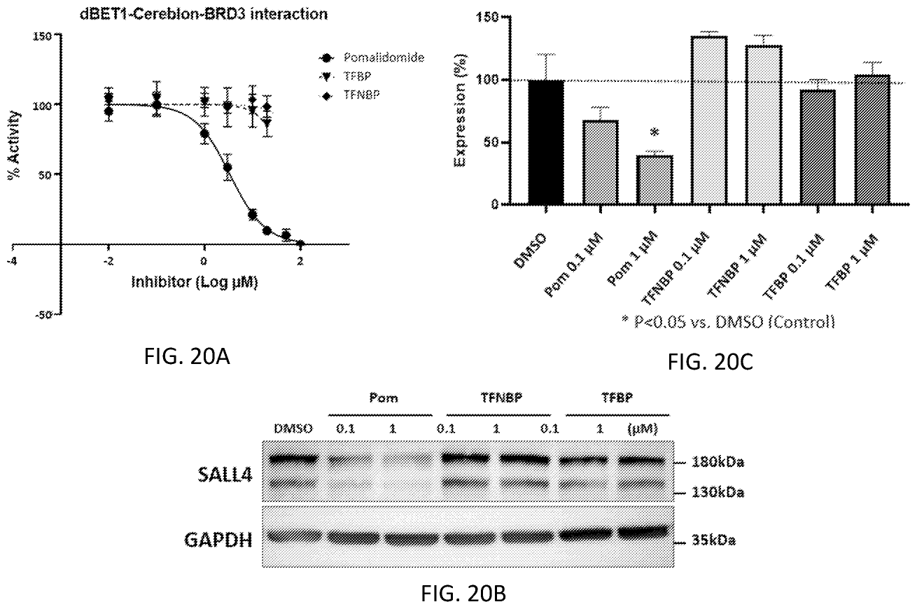

Studies have shown that a key teratogenicity mechanism of thalidomide and analogue compounds derives from their ability to bind cereblon and, consequently, elicit the ubiquitination and decreased expression of key downstream neosubstrates, particularly SALL4 (Chamberlain et al., Drug Discovery Today Technol 2019. 31:29-34; Ito et al., Int J Hematol 2016, 104:293-299; Stewart, Science 2014, 243:256-257; Vargesson, J Hand Surg Eur Vol 2019, 44:88-95; Matyskiela et al., Nat Srtuct Mol Biol. 2020, 27:319-22). Considering the partial structural similarity of compound 14 and compound 16 with thalidomide-analogue drugs, their interaction with cereblon was analyzed through a cereblon/BRD3 binding FRET assay. Compound 14 and compound 16 were not able to bind cereblon (IC₅₀ values of 53.45 μM and >100 μM, respectively (aligning with background non-binding), as compared to pomalidomide (IC₅₀ 3.36 μM) (FIG. 20A, Table 11). Whereas 1 μM pomalidomide induced a decrease in expression levels of SALL4, treatment with compound 14 and compound 16 in Tera-1 cells at 0.1 μM and 1 μM concentrations did not statistically affect (p>0.05) this key neo-substrate involved in teratogenicity, potentially representing a safer alternative to thalidomide-analogues (FIGS. 20B, 20C).

TABLE 11
Cereblon binding (IC₅₀ value)

| Compound | IC₅₀ (μM) |
|---|---|
| Pomalidomide | 3.36 |
| Compound 14/TFBP | 53.45 |
| Compound 16/TFNBP | >100 |

### DISCUSSION AND CONCLUSION

TBI is a leading cause of death and disability worldwide, representing an exceptional challenge in terms of socioeconomic costs. Survivors of moderate to severe TBI often experience a series of physical, behavioral and cognitive deficits, leading to serious consequences for the individuals and their caregivers (Langlois et al., J Head Trauma Rehabil 2006, 21:375-8; Thurman et al., J Head Trauma Rehabil. 1999, 14:602-15). Currently, there are no approved pharmacological treatments able to prevent the development of long-term symptoms in TBI patients.

In this study, the novel IMiD compound 14 (TFBP) was evaluated in a mouse model of CCI-induced TBI, representative of a moderate TBI in humans. After observing that compounds 14 and 16 were able to mitigate several markers of inflammation in different in vitro and in vivo experimental models, including in brain, compound 14 was evaluated in a mouse model of TBI. Systemic administration of compound 14 resulted in a reduction of neuronal cell death associated with the injury, as well as a decrease in microglial activation; these neuroprotective and anti-inflammatory actions resulted in the more rapid mitigation of motor functional deficits induced by CCI injury.

The neuropathology of TBI is commonly described as a 2-phase process; while very little can be done to intervene pharmacologically on the first mechanical stage of the injury, the second phase comprises a series of long-lasting processes that could be considered as potential therapeutic targets. These secondary processes, which include neuroinflammation, excitotoxicity, mitochondrial dysfunction and axon degeneration, do not affect just the specific area for mechanical damage, but are also found in brain regions remote from the initial injury, contributing to a widespread damage (Acosta et al., Plos One 2014, 9: e90953). Neuroinflammation, in particular, has been proven to play a major role in the development and progression of TBI. Microglial activation starts early after head injury, as demonstrated in preclinical and human studies (Chiu et al., J Neurosci Methods 2016, 272:38-49; Ramlackhansingh et al., Ann Neurol. 2011, 70:374-83). Consistently with glial activation, CSF and brain levels of pro-inflammatory cytokines such as TNF-α, IL-1β, IL-6, IFN-γ and IL-5 have been found to be elevated in several experimental models of TBI, as well as in TBI patients, withing hours after the injury (Ross et al., Brit J Neurosurg. 1994, 8:419-25; Baratz et al., J Neuroinflamm. 2015, 12:45; Frugier et al., J Neurotrauma 2010, 27:497-507; Woodcock and Morganti-Kossman, Front Neurol. 2013, 4:18). In particular, TNF-α is known to play a central role in the TBI-mediated inflammation. Numerous studies showed how TNF-α is involved in driving the activation of glial cells (Liddelow et al., Nature 2017, 541:481-487; Kuno et al., J Neuroimmunol. 2005, 162:89-96; Abd-El-Basset et al., AIMS Neurosci. 2021, 8:558-584); this event, in turn, leads to a further increase in the production and release of TNF-α as well as other inflammatory cytokines and mediators, such as reactive oxygen species (ROS), nitric oxide, glutamate and also promotes the complement cascade, which worsens neuronal damage and death (Dinet et al., Front Neurosci. 2019, 13:1178; Woodcock and Morganti-Kossmann, Front Neurol. 2013, 4:18). On this basis, it is not surprising that many recent studies are focusing on addressing neuroinflammation as a potential therapeutic target in brain injury.

The chemical structures of compounds 14 and 16 are characterized by the presence of a tetrafluorinated phthalimide group that provides these compounds the core element of immunomodulatory imide drugs (IMiDs), such as thalidomide and derivatives. One of the primary mechanisms of actions through which this class of drugs exerts its anti-inflammatory effect, is the ability to lower levels of TNF-α (Sampaio et al., J Exp Med. 1991, 173:699-703; Muller et al., J Med Chem. 1996, 39:3238-40; Quach et al., Leukemia 2010, 24:22-32). In particular, IMiDs bind the 3′-untranslated region (UTR) of TNF-α mRNA through their phthalimide groups, altering its transcription and ultimately affecting protein translation (Moreira et al., J Exp Med. 1993, 177:1675-80; Rowland et al., Immunol Lett. 1999, 68:325-32). In agreement with their ability to lower TNF-α, thalidomide-analogs in TBI models provide an anti-inflammatory effect, by mitigating glial activation and reducing the expression of not only TNF-α, but several pro-inflammatory mediators (Wang et al., J Neuroinflamm. 2016, 13:228; Huang et al., In J Mol Sci. 2021, 22:8276; Lin et al., eLife 2020, 9: e54726). In models of CCI, the thalidomide analog 3,6′-dithiothalidomide (3,6′-DTT) inhibited microglial activation and downregulated expression of TNF-α mRNA at 8 hours after the injury, as well as IL-1β and IL-6 (Batsaikhan et al., Int J Mol Sci. 2019, 20:502). The more recently developed analog 3,6′-dithiopomalidomide (3,6′-DP), as well as its precursor pomalidomide, demonstrated a comparable anti-inflammatory effect in a similar model of CCI; treatment with both compounds resulted in reduced glial activation and expression of pro-inflammatory cytokines, including TNF-α, IL-1β and IL-6 (Wang et al., J Neuroinflamm. 2016, 13:228; Lin et al., eLife 2020, 9: e54726). The anti-inflammatory effect of these drugs is accompanied by a reduction of TBI-induced neuronal death, as indicated by the reduction in the lesion volume, and improvement of the behavioral outcome (Baratz et al., J Neuroinflamm. 2015, 12:45; Wang et al., J Neuroinflamm. 2016. 13:228; Lin et al., eLife 2020, 9: e54726; Batsaikhan et al., Int J Mol Sci. 2019, 20:502; Huang et al., In J Mol Sci. 2021, 22:8276).

Considering the partial structural similarity of compound 14 with thalidomide and thalidomide-like drugs, its anti-inflammatory activity was investigated in a CCI model of moderate TBI. As initial screening, compound 14 and its close analog compound 16 were tested in RAW 264.7 mouse cell cultures challenged with LPS. Both agents were well tolerated with no statistically significant changes in cell viability following the administration of the compounds at nanomolar concentrations. This resulted in a dose-dependent decline in levels of nitrite and TNF-α, two classical markers of inflammation whose expression was markedly elevated in the LPS group compared to the control group (data not shown). The anti-inflammatory activity of both compound 14 and compound 16 was confirmed in an in vivo model of inflammation in rats, where compound 14 and compound 16 mitigated a LPS-induced increase of TNF-α in plasma and cortex, and IFN-γ and IL-5 in plasma. Moving to the CCI model of TBI, an evaluation of markers of inflammation at 2 weeks after the injury showed that post-injury treatment with compound 14 was able to mitigate microglial activation. Based on changes in cell morphology, our results suggest that animals treated with compound 14 after TBI displayed an increased expression of microglial M2 phenotype compared to the CCI-alone group; microglial and astroglial proliferation was also measured, but no statistically significant differences among groups were observed (data not shown). In relation to thalidomide-analogs tested in CCI models of TBI, the anti-inflammatory effect of compound 14 was accompanied by a neuroprotective action, which was observed in particular as a reduction of lesion volume. Ultimately, the protective and anti-inflammatory action of compound 14 resulted in a more rapid mitigation of the motor and coordination impairment in the CCI-challenged mice.

The question arises as to why the motoric behavioral changes here largely resolve 2 weeks after the CCI whereas the histological and biochemical changes in the study persist longer. There is much preclinical and clinical literature on compensatory central motor mechanisms after brain injury, involving both the ipsilateral hemisphere distal to the injury and the contralateral hemisphere. Although this literature is too extensive to be detailed here, the role of the contralateral hemisphere is most dramatically indicated by studies in the rodent split brain preparation, where post injury motor compensation is eliminated after section of the corpus callosum (Lienhard, “Roger Wolcott Sperry (1913-1994),” Embryo Project Encyclopedia 2018, ISSN: 1940-5030). Moreover, although the analogy is somewhat flawed, 2 weeks in a 24 month old mouse is equivalent to about 70 weeks in a 70 year old human. Interestingly, clinical literature suggests there is significant resolution of some about 10-12 months after a TBI.

Another issue can involve the types of motor behavior analyzed here. Both ambulation on a narrow beam and walking on a treadmill involves balance as well as simple movement parameters. The clinical literature suggests while simple walking can often recover in months after injury, recovery of balance which requires both vestibular and limb proprioceptive function, is often delayed (Row et al., J Neurotrauma, 2019, 36 (16): 2435-2442).

Summarizing, our in vitro and in vivo studies demonstrated a promising effect of compound 14 in mitigating neuroinflammation and eliciting a protective action in a TBI model that aligns with moderate injury in humans, warranting further investigation as a potential candidate for neurodegenerative conditions with an inflammatory component.

The novel IMiD compound 14 provides neuroprotective actions by reducing cortical neuronal loss and improving the behavioral outcome in a CCI mouse model of moderate TBI. Additionally, treatment with the compound mitigates injury-related changes in microglia morphology; the anti-inflammatory potential is also confirmed by its ability to reduce levels of pro-inflammatory cytokines in plasma and cortex in a classical LPS rat model of inflammation. Of particular note, unlike thalidomide and clinically approved IMiDs such as pomalidomide, compounds 14 and 16 do not bind to cereblon and, importantly, do not lower the levels of the key neosubstrate SALL4 that is considered to largerly underpin the tertatogenicity of this drug class (Chamberlain et al., Drug Discovery Today Technol 2019, 31:29-34; Vargesson, J Hand Surg Eur Vol 2019, 44:88-95; Matyskiela et al., Nat Srtuct Mol Biol. 2020, 27:319-22)

### Example 6

### Fluoro-3,6′-Dithiopomalidomide as a Treatment for Traumatic Brain Injury

Traumatic brain injury (TBI) is a leading cause of death and disability in children and adults. Sixty-nine million individuals worldwide are estimated to be affected by TBI each year (Dewan et al., J Neurosurg 20118, 1-18). TBI creates damage to the brain by shearing forces, objects direct contact or penetration (Archer, Neurtox Res 2012, 21:418-434; Webb et al., NeuroRehabilitation 2014, 34:625-636). Post-injury impairments including aspects of cognitive, motor, mood have been mentioned after TBI, along with deficits in gait and locomotion (Williams et al., Arch Phys Med Rehabil 2009, 90:587-593). Besides the direct injury and functional impairments induced by TBI, accumulating epidemiological data showing that there is a strong link between TBI and chronic neurodegenerative disorders, such as Parkinson's disease (PD) and Alzheimer's disease (AD) (Crane et al., JAMA Neurol 2016, 73:1062-1069; Shahaduzzaman et al., Med Hypotheses 2013, 81:675-680; Wong et al., Crit Rev Clin Lab Sci 2013, 50:103-106; Fann et al., Lancet Psychiatry 2018, 5:424-431; Gardner et al., Neurology 2018, 90: e1771-e1779). The obvious hallmarks of neuroinflammatory response after TBI are blood-brain barrier (BBB) disruption, edema, the activation and relocation of astrocytes and microglia, the production and release of cytokines, chemokines, and the recruitment of blood-derived leukocytes, like neutrophil infiltration into local area (Morganti-Kossmann et al., Curr Opin Crit Care 2002, 8:101-105; Webster et al., J Neuroinflammation 2017, 14:10; Chiu et al., J Neurosci Methods 2016, 272:38-49; Loane et al., Trends Pharmacol Sci 2010, 31:596-604; Banjara et al., Int J Inflam 2017, 2017:8385961). Both clinical and pre-clinical studies have indicated a profound expression of several cytokines after TBI, such as tumor necrosis factor (TNF-α), transforming growth factor-β (TGF-β), and interleukin-1β (IL-1β), -6, and -10, Keratinocyte chemoattractant (KC)/human growth-regulated oncogene (GRO) chemokines (KC/GRO, CXCL1), Interferon gamma (IFN-γ), following downstream signaling of nuclear factor Kappa-light-chain-enhancer of activated B cells (NF-κB) pathway (DeKosky et al., Nat Rev Neurol 2013, 9:192-200; Morganti-Kossmann et al., Curr Opin Crit Care 2002, 8:101-105; Nonaka et al., J Neurotrauma 1999, 16:1023-1034; Scherbel et al., PNAS U.S.A. 1999, 96:8721-9726; Sherwood et al., Crit Care Med 2000, 28:1221-1223; Clark et al., Pharmacol Ther 2010, 128:519-548; Woodcock et al., Front Neurol 2013, 4:18; Dalgard et al., Front Mol Neurosci 2012, 5:6).

The efficacy of fluoro-3,6′-dithiopomalidomide (compound 30, F-3,6′-DP) was assessed in several in vitro and in vivo models of neuroinflammation and TBI. In particular, compound 30 was evaluated to determine whether it could efficiently reduce inflammatory mediators in cellular and rodent models of lipopolysaccharide (LPS) created inflammatory environment, and to determine its efficacy on mitigating behavioral, histological impairments, neuroinflammation caused by TBI.

### Methods

Animal studies: All rodents were housed at 25° C. in a 12 h light/12 h dark cycle and given free access to food and water. All efforts were made to minimize animal suffering and to decrease the number of animals used by integrating the outcome measures from our previous studies and the statistic power analysis. All procedures used in this study were fully approved by the Institutional Animal Care and Use Committees (Intramural Research Program, National Institute on Aging, NIH (protocol No. 488-TGB-2022)).

Systemic and brain LPS anti-inflammatory studies: Adult male Fischer 344 rats (approx. 150 g weight) were randomly assigned across groups, and then given compound 30 (14.78 or 29.57 mg/kg (equimolar to 12.5 and 25 mg/kg thalidomide), i.p., dissolved in 1% carboxymethyl cellulose (CMC) in normal saline) or vehicle, 60 min before either LPS (1 mg/kg, Sigma, St Louis, MO, in normal saline, 0.1 ml/kg i.p.) or vehicle. These concentrations of the drug were chosen for the in vivo evaluation of compound 30 as they are equimolar to, or are less than, doses of thalidomide and analogs that have verified to be well-tolerated in previous studies and are of translational application to humans. Four hours after LPS injection, animals were euthanized, and blood and brain tissue were collected and placed on wet ice. Plasma was quickly separated from blood by centrifuge at 10,000 g. 5 min, 4° C., and brain regions of cortex, hippocampus and the rest of brain were dissected into separate vials on wet ice, then stored at −80° C. Brain tissues were then sonicated in a TRIS based lysis buffer (Mesoscale Discovery) with 3× protease/phosphatase inhibitors (Halt™ Protease and Phosphatase Inhibitor Cocktail, Thermo Fisher Scientific), were then centrifuged at 10,000 g, 10 min, 4° C., and protein concentrations were measured by Bicinchoninic acid assay (BCA, Thermo Fisher Scientific). Rat plasma, cerebral cortical and hippocampal samples were then analyzed by multi-proinflammatory cytokines ELISA (V-PLEX Proinflammatory Panel 2 Rat Kit, Mesoscale Discovery), following the manufacturer's protocol. The processes and dosages of the experiment of drug treatment effects on LPS rat model was made on the basis of our previous studies.

In Vivo Model of TBI: TBI studies were conducted in 8-week-old male C57/BL6 mice (25-30 g; n=20), Jackson Laboratory, Bar Harbor, Maine USA. Mice were randomly assigned to four groups prior surgery, CMC+Sham, CMC+TBI, compound 30 (Low dose (LD), 14.78 mg/kg, prepared in 1% CMC in PBS)+TBI, compound 30 (High dose (HD), 29.57 mg/kg)+TBI, and evaluated the effects of compound 30 on TBI. Mice were assessed for gait function and motor coordination, balance function. Animals were subsequently evaluated for cellular changes using histological and immunohistochemistry staining.

Mice were anesthetized with 2.5% tribromoethanol (Avertin: 250 mg/kg; Sigma, St. Louis, MO, USA)) and placed in a mouse stereotaxic frame (Kopf Instruments, Tujunga, CA, USA) and fixed by ear bars and incisor bar. Under sterile procedures, the skin was retracted and a 5-mm square craniectomy was executed over right motor cortex at the posterior corner between the bregma and sagittal sutures. The skull was carefully removed by a drill to avoid damaging the dura underneath. The process of craniectomy proceeded without having the temporalis muscle impaired. The CCI device, Impact One (Leica Biosystems Inc., Buffalo Grove, IL, USA), consists of an electromagnetic impactor that allows alteration of injury severity by controlling contact velocity and the depth of cortical deformation independently. Prior to impact administration, the tip of 3-mm flat impactor was angled and kept perpendicular to the exposed cortical surface. The contact velocity was set at 5 m/s, dwell time was set at 0.2 s and deformation depth was set at 2 mm to produce moderate to severe TBI. After the impact, we used sterile cotton tipped applicators to clean up the area around injury site and then closed the wound by surgical needle (Mani Inc., Utsunomiya, Tochigi, Japan) and surgical suture (Ethicon Inc., Somerville, NJ, USA). Sham animals were anesthetized, and followed the same procedure as TBI mice without the impact. During surgery and recovery, the core body temperature of all mice was maintained at 36-37° C. using either heat pad or heated chamber. Mice will return to their home cage after they had woken up form anesthesia. Mice were then given compound 30 (14.78 or 29.57 mg/kg in 0.1 ml/10 g body weight) or CMC vehicle by i.p. injection, with the first injection administered 45 minutes after injury and the second injection on the next day

Beam walk test: TBI-induced impairments in motor coordination were evaluated by beam walk test (BWT). Mice have an intrinsic tendency staying in a darkened enclosed environment, comparing to an open illuminated field. Each mouse was placed in darkened goal box for a 2 min habituation and mouse was then moved to the other (light) end of the beam to start the trial. Time spends and the number of footfalls during crossing the beam were recorded at baseline (PRE), 1 and 2 weeks after TBI, with the caveat that total time was not to exceed 30 s. The dimensions of the beam were 1.2 cm (width)×91 cm (length). The time taken for each animal to traverse the beam to reach the dark goal box, and the number of ipsilateral and contralateral foot falls were documented. Five trials were recorded for each animal before CCI and 1 and 2-weeks after CCI. The mean times to traverse the beam were calculated, and a plot was generated to evaluate treatment effects on beam walk times and foot falls; these times were then used for statistical analysis.

Gait analysis: DigiGait was used to analyze gait parameters per the manufacturer's protocol (Mouse Specifics, Inc.) at baseline (PRE), 7 days (1 Wk), and 14 days (2 Wks) post injury. At each timepoint, each animal was moved to the testing chamber and allowed to acclimate for 2 minutes to the new environment while software was set up and bumpers adjusted to maintain the animal in field of view. The treadmill was initially started at 5 cm/s and the animal was allowed to run for 1 minute, then give a break for 1 minute. The speed was gradually increased to the testing speed of 15-20 cm/s at which time recording was initiated. Once 3-5 seconds of constant stepping was captured, the treadmill and the recording were stopped and the animal was returned to its home cage.

Animals falling behind off camera, feet stepping sideways outside of the camera field of view, brief stopping of gait or running in squiggles with sharp turning left and right, were excluded from the video. When all animals finished their trials, the videos were analyzed with the DigiGait software evaluating stride duration, length, paw angle, stance, etc. (Mouse Specifics, Inc.). These parameters were evaluated for all four limbs. Parameters included brake time (time duration of the braking phase (initial paw contact to maximum paw contact, commencing after the swing phase)), % of brake phase (percent of the total stride duration that the paw is in the braking phase), propel time (time duration of the propulsion phase (maximum paw contact to just before the swing phase)), % of propel phase (percent of the total stride duration that the paw is in the propulsion phase) and paw angle variability (the standard deviation of the paw angle for the set of strides recorded). All assessments were made by an investigator blinded to group.

Histological analysis: Fixation and sectioning-Animals were anesthetized with 2.5% tribromoethanol, Avertin (Sigma, St. Louis, MO, USA) and perfused transcardially with 0.9% saline and 4% PFA in 0.1 M phosphate buffer (PB, pH 7.2). Brains were removed and post-fixed for 1 day in 4% PFA and sequentially transferred to 20% and 30% sucrose in 0.1 M PB until the brain sank. The brains were cut into 25-μm sections on a cryostat (Leica Biosystems Inc., Buffalo Grove, IL, USA). Every seventh section was selected from a region spanning from striatum to hippocampus.

Quantification of brain lesion and lateral ventricle size in TBI animals-One set of post-TBI 2-week brain sections (25 μm) were mounted on slides. The sections were then stained in 10% Giemsa KH₂PO₄ buffered solution (pH 4.5) for 30 min at 40° C. After a brief rinse, slides were de-stained, differentiated, and dehydrated in absolute ethanol. Thereafter, the sections were cleared in xylene and then coverslipped. Slides were scanned in an All-in-One Fluorescence Microscope BZ-X710 (Keyence Corporation of America, Itasca, IL, USA), and brain image areas were quantified using ImageJ 1.52q software (National Institutes of Health, Bethesda, MD, USA). The calculation formula for contusion volume size and lateral ventricle size was as follows: Σ (area of contralateral hemisphere−area of ipsilateral hemisphere)/Σ area of contralateral hemisphere; Σ area of ipsilateral lateral ventricle/Σ area of contralateral lateral ventricle. There were 9 brain sections from each mouse counted, with regions starting from bregma 0.86 mm to −1.46 mm.

Immunofluorescence: 4 brain sections per mouse were incubated with blocking buffer (4% Bovine Serum Albumin, Sigma, St. Louis, MO, USA) for 1 hr. A series of primary antibodies were prepared in the blocking buffer and the sections were incubated in the solution overnight. The antibodies used were goat anti-GFAP (Glial Fibrillary Acidic Protein) (1:500; Abcam, Cambridge, MA, USA), or rabbit anti-Iba1 (Ionized calcium binding adaptor molecule 1) (1:500; FUJIFILM Wako Pure Chemical Corporation, Richmond, VA, USA). After incubation with primary antibody, the sections were washed and incubated for 3 hr at room temperature in diluted secondary antibody prepared with blocking solution ((secondary antibody conjugated with Alexa 488 or 555 (1:500; Thermo Fisher Scientific, Waltham, MA, USA)). The sections were then washed with 0.1 M PB (pH 7.2), mounted with Antifade Mounting Medium with DAPI (Vector, Burlingame, CA, USA) and cover-slipped. A series of 4 images per mouse brain were taken using a Laser Scanning Microscope (Zeiss 710, Oberkochen, Germany). Cell numbers of each image were counted using ImageJ 1.52q software (National Institutes of Health, Bethesda, MD, USA).

Immunofluorescence analysis and quantification: Iba1-positive microglia and GFAP-positive astrocytes were identified with a ×40 oil magnification objective. For each mouse, four to six fields of cortex were captured from both ipsilateral and contralateral hemispheres. The immunoreactive (IR) cell numbers of microglia and the total area of GFAP IR in each field were quantified by NIH software ImageJ 1.52q. Observers were blinded as to treatment group analysis.

In relation to Iba1 immunostaining, microglial cells were subclassified into morphological subtypes in line with prior studies, as microglia morphology is considered highly representative of their functional state. These subtypes included ramified and intermediate type microglial cells as well as amoeboid and round types. Morphometric parameters were analyzed using MotiQ, a fully automated analysis software. MotiQ was developed as an ImageJ plugin in Java and is publicly available (https://github.com/hansenjn/MotiQ). The microglial ramification index is the ratio of cell surface area and surface area of a perfect sphere with the same volume as the analyzed cell. The ramification index is a unit-free parameter for the complexity of the cellular shape. A ramification index of 1 corresponds to a perfectly round cell without processes. The more the cell differs from a perfectly round shape, i.e. the more branches the cell possesses, the higher is its 3D ramification index. All segmentation and quantification were performed on maximum intensity projections of 3D image data.

### Cellular Studies

Cereblon binding and neo-substrate assays: A bead-based AlphaScreen technology was adopted for cereblon binding, with minimal modifications from the manufacturer's protocol (BPS Bioscience catalog no. 79770). Compound 30 or pomalidomide was incubated with reaction mixtures including cereblon/DNA damage-binding protein 1-Cullin 4a-ring-box protein 1 complex (CRBN/DDB1-CUL4A-Rbx1, 12.5 ng) and bromodomain-containing protein 3 (BRD3) (6.25 ng) in an Optiplate 384-well plate (PerkinElmer catalog no. 6007290). After 30 min of incubation with shaking at room temperature, AlphaLISA anti-FLAG Acceptor and Alpha Glutathione Donor beads (PerkinElmer) were sequentially added and then incubated for 1 h at room temperature for each of the added chemicals. Alpha counts were thereafter read on a Synergy Neo2 (BioTek) for the analysis. The relative activity of the alpha signal was calculated after subtraction of the “blank value” from all readings, and the value of the vehicle group was then set as 100%.

The effect of compound 30 activity on neo-substrates was evaluated in both MM1.S (myeloma) and Tera-1 cell lines. Specifically, MM1.S cells were obtained from ATCC (Manassas, VA, USA), grown in RPMI media supplemented with 10% FBS, penicillin 100 U/mL, and streptomycin 100 mg/mL, and maintained at 37° C. and 5% CO2. MM1.S cells were treated with 1 μM of pomalidomide or compound 30 for 24 h; thereafter, the cell lysates were prepared for Western blot analysis, as described previously (Tsai et al., Pharmaceutics 2022, 14:950). Tera-1 cell lines were obtained from Korean Cell Line Bank (catalog no. 30105; Seoul, Korea) and grown in Dulbecco's modified Eagle's medium (DMEM), supplemented with 10% FBS, penicillin 100 U/mL and streptomycin 100 μg/mL, and maintained at 37° C. and 5% CO2. Tera-1 cells were treated with pomalidomide or compound 30 (0.01, 0.1 and 1 μM) for 4 h, and their cell lysates were prepared for the Western blot analysis, as described previously (Lin et al., eLife 2020, 9:354726).

For Western blot analysis, total proteins were extracted using RIPA buffer (ThermoFisher Scientific, Waltham, MA, USA) containing Halt Protease Inhibitor Cocktail (ThermoFisher Scientific). Thereafter, the proteins were separated by gel electrophoresis and then transferred to polyvinylidene difluoride (PVDF) membranes (ThermoFisher Scientific), as described previously [74]. The following primary antibodies were used: (i) anti-Ikaros antibody (catalog no. 9034; 1:1000; Cell Signaling Technology, Danvers, MA), (ii) anti-Aiolos antibody (catalog no. 15103; 1:1000; Cell Signaling Technology), (iii) anti-SALL4 antibody (catalog no. ab29112; 1:1000; Abcam, UK), and (iv) anti-GAPDH antibody (catalog no. ab8245; 1:5000; Abcam, UK). After incubation at 4° C. overnight, the following HRP-conjugated secondary antibodies were used: (i) goat anti-rabbit IgG (ThermoFisher Scientific) for Ikaros and Aiolos, and (ii) goat anti-mouse IgG (ThermoFisher Scientific) for SALL4 and GAPDH. GAPDH, a protein that is generally expressed in all eukaryotic cells, was used as an internal control against which the other protein expression levels were compared. Antigen-antibody complexes were detected using enhanced chemiluminescence (ThermoFisher Scientific, iBright CL1500).

Cellular Studies in RAW Cells: Mouse RAW 264.7 cells, originally acquired from ATCC (Manassas, VA), were grown in DMEM supplemented with 10% fetal calf serum (FCS), penicillin 100 U/mL and streptomycin 100 μg/mL, and were maintained at 37° C. and 5% CO₂. The cells were grown in accordance with ATCC guidelines, as previously described (Tsai et al., Pharmaceutics 2022, 14:950). On the day of the study, RAW 264.7 cells were challenged with LPS (Sigma, St. Louis, MO: serotype 055: B5) at a final concentration of 60 ng/mL. This LPS concentration routinely induces a submaximal rise in both TNF-α and nitrite levels without a loss of cell viability. Such a submaximal rise is useful for assessing whether the addition of an experimental drug can either lower or further elevate the levels of TNF-α and nitrite. In a drug pretreatment paradigm, either compound 30 (0.6-60 μM) or vehicle (Veh), n=3-4, was administered 60 min prior to LPS challenge. At 24 h following the addition of LPS, conditioned media was harvested, and both secreted TNF-α protein (ELISA MAX™ Deluxe Set Mouse TNF-α, catalog no. 430904, BioLegend, San Diego, CA, USA) and nitrite levels (Fluorometric Assay Kit, Abnova, catalog no. KA1344, Walnut, CAUSA) were quantified as recommended by the manufacturers. Fresh media were replaced in the wells, and cell viability was thereafter evaluated with a CellTiter 96 Aqueous One Solution Cell Proliferation Assay (Promega, Madison, WI, USA). For cell culture studies, compound 30 was prepared immediately prior to use in 100% DMSO and then added to cell culture media at a dilution of greater than 200-fold to provide the desired concentrations; control-/veh-treated cells were subjected to the exact same procedure, but without the addition of compound 30.

Docking pockets and predictions of compound 30 and thalidomide structural analog interacitons with cereblon: Evaluations of potential docking pockets and docking predictions for S enantiomeric forms of compound 30, thalidomide and pomalidomide, on the structure of cereblon were analyzed. Prospective drug docking pockets were initially determined to support the computation of the pharmacophore engagement by the molecules using automated software (Martínez-Rosell et al., J Chem Inf Model. 2017, 57:1511-6). This was followed by a cavity-based blind drug docking prediction utility (Jiménez et al., Bioinformatics 2017, 33:3036-42) to evaluate the attributes of the drug docking prediction of these IMiDs. The drug docking pockets and the binding differences between compound 30, thalidomide and pomalidomide in cereblon were determined for the best scoring attributes of these chemical agents. Briefly, the crystal structure of human cereblon in complex with DDB1 and lenalidomide (4TZ4: https//www.rcsb.org/structure/4TZ4, accessed on 27 Jul. 2022) was downloaded in PDB format from the PDB database. Chain C (human cereblon) was isolated from the rest of the crystal structure complex and was prepared for docking predictions for the S enantiomeric forms of thalidomide, pomalidomide, and compound 30. This enantiomer was selected because prior X-ray crystallographic studies have indicated that the S rather than R enantiomeric form of classic thalidomide-like drugs more optimally binds cereblon (Boichenko et al., ACS Omega 2018, 3:11163-71; Chamberlain et al., Drug Discov. Today Technol. 2019, 31:29-34), albeit molecular modeling computational data does not necessarily simulate or fall in line with all empirical data from prior X-ray crystallographic studies. The automated server at Playmolecule, which uses a software DeepSite (Martínez-Rosell et al., J Chem Inf Model. 2017, 57:1511-6; Jiménez et al., Bioinformatics 2017, 33:3036-42) to determine the core binding sites, was used to evaluate potential interactions between compound 30, thalidomide and pomalidomide with human cereblon. The S chiral forms of thalidomide and pomalidomide were downloaded and evaluated as this enantiomeric form, as noted above, is reported to have more potent cereblon binding (Boichenko et al., ACS Omega 2018, 3:11163-71; Chamberlain et al., Drug Discov. Today Technol. 2019, 31:29-34; Asatsuma-Okumura et al., Pharmacol. Ther. 2019, 202:132-9; Matyskiela et al., Nat. Chem. Biol. 2018, 14:981-7). Automated docking software (Liu et al., Acta Pharmacol. Sin. 2020, 41:138-44) was used to investigate potential similarities and differences in the pharmacophore pockets engaged by the three drugs. Briefly, two files were uploaded that included the C-chain (human cereblon) of PDB ID 4TZ4 without lenalidomide and damaged DNA binding protein 1 (DDB1) and the drugs individually in their PDB formats to the Docking server (Ibid.). For docking pocket predictions, the results appear as the number of preferential pockets determined by their relevant scores. The results of docking were collected with individual Vina scores, cavity sizes, docking centers, poses, and sizes of predicted cavities for the drugs noted above. The resulting drug-cereblon complexes were visualized using the drug discovery studio visualizer software BIOVIA (Martínez-Rosell et al., J Chem Inf Model. 2017, 57:1511-6). The top binding modes of compound 30, pomalidomide and thalidomide with Vina scores with their principal binding cavities were selected for comparison of the binding pose of the drugs and the pockets occupied by them.

### Results

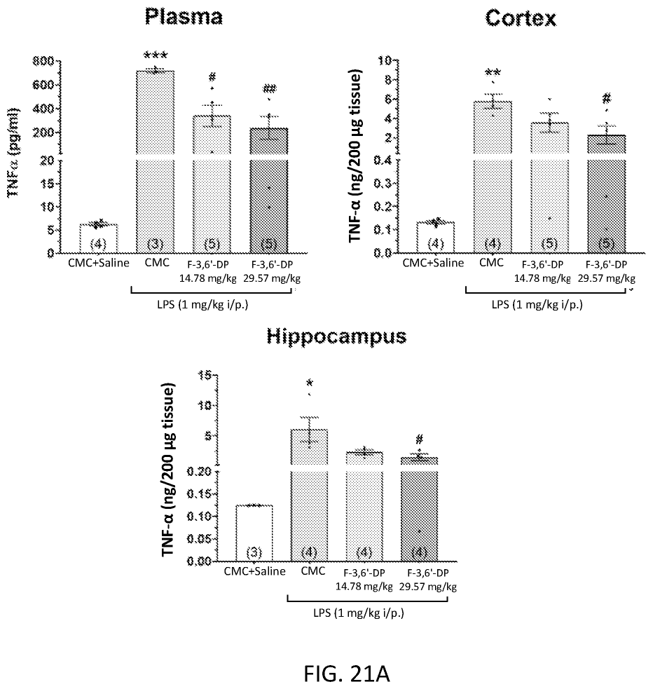

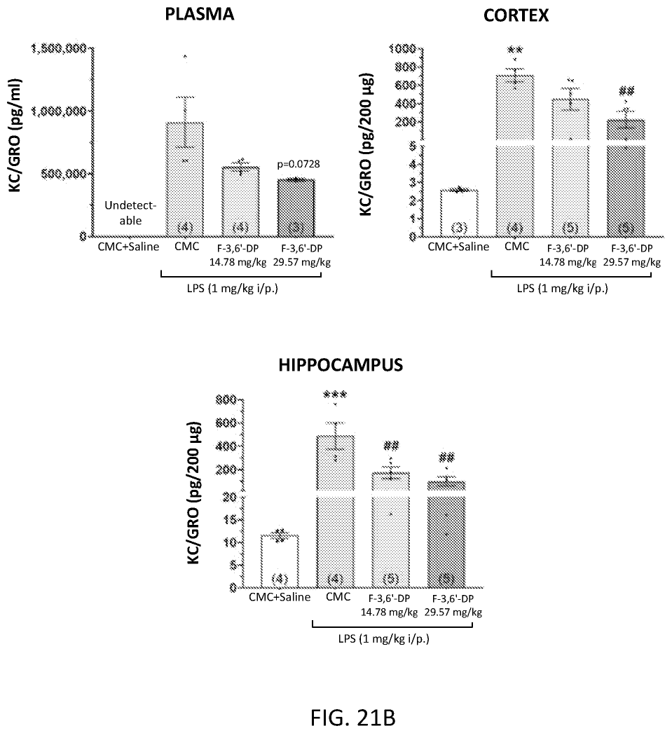

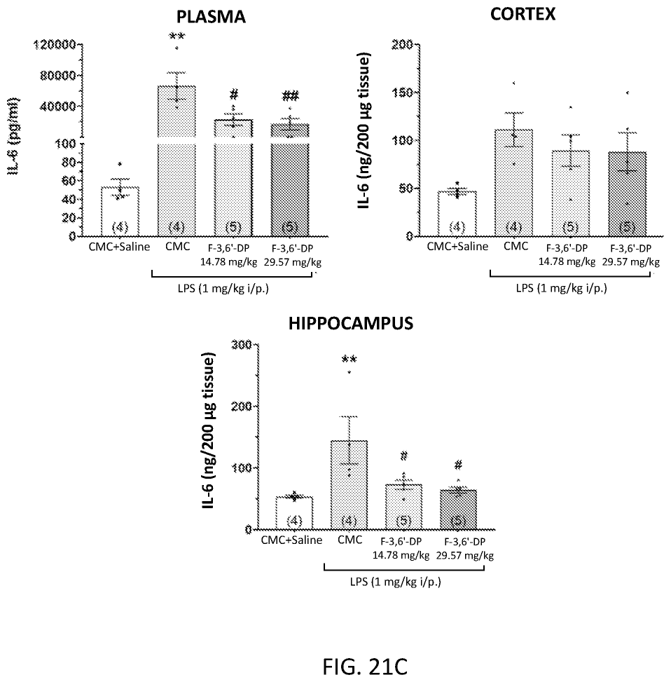

Compound 30 reduced the expression of pro-inflammatory cytokines, TNF-α, IL-6 and chemokine KC/GRO (CXCL1) induced by LPS in rat. To evaluate the anti-inflammation activities of compound 30 in vivo, the lipopolysaccharide (LPS) rat model was utilized as a screening platform. Systemic treatment of LPS can induce significant amount of TNF-α, KC/GRO (CXCL1), IL-6 in plasma, cortex and hippocampus of brain (FIGS. 21A-21C). Systemic administration of compound 30 (29.57 mg/kg i.p.) can significantly reduce the level of TNF-α in both plasma and brain (FIG. 21A), KC/GRO in brain (FIG. 21B) and IL-6 in plasma, cortex, and hippocampus of brain (FIG. 21C).

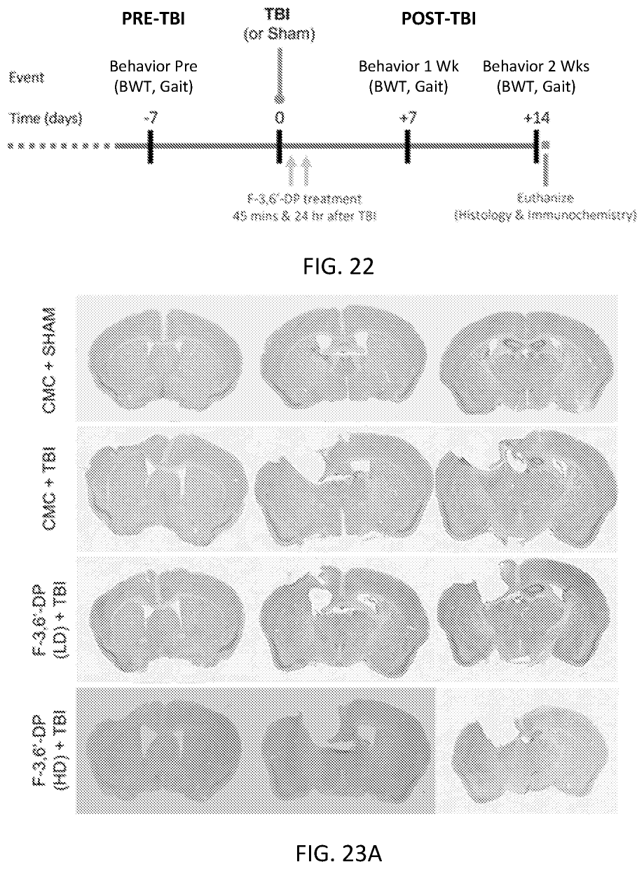

Compound 30 ameliorated behavioral deficits and contusion volume produced by TBI. As an assessment of whether the anti-inflammatory properties in LPS rat model benefit to disease animal model, the ability of compound 30 to ameliorate TBI induced impairments was measured in mouse TBI model. As shown in FIG. 22, mice received control cortical impact to mimic TBI at day 0, and behavioral assessments were conducted at 1 week before TBI (day −7), 1 and 2 weeks after TBI (day +7, +14). Either compound 30 or vehicle (CMC) was given to mice at 45 minutes and 24 hours after TBI, and mice were euthanized at 2 weeks after TBI for histology and immunostainings.

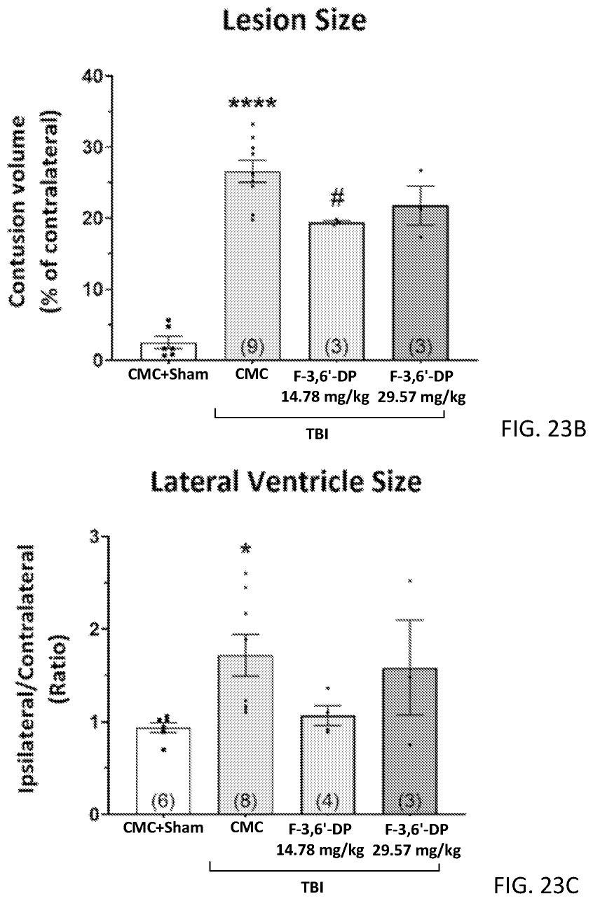

Histology staining was performed to evaluate the brain tissue loss and lateral ventricle size enlargement induced by TBI. The contusion volume is demonstrated as a percentage of contralateral volume for each group and lateral ventricle size is presented as ratio of ipsilateral versus contralateral size. After TBI, there was a significant tissue loss observed in ipsilateral cortex (FIG. 23A), comparing with the sham group (p<0.0001) (FIG. 23B), and there was a significant reduction of the lesion size in compound 30 low dose treatment group (14.78 mg/kg) comparing to vehicle (CMC) group (p<0.05) (FIG. 23B).

The size of lateral ventricle was also measured as an evaluation of the changes of intracranial cerebrospinal fluid (CSF) in mice brain after TBI. The enlargement of ventricle size is a typical marker in clinical after TBI (McKee et al., Handb Clin Neurol. 2015, 127:45-66). Significant elevation of the lateral ventricle size was observed in the ipsilateral side of brain after TBI (p<0.05) (FIG. 23C). However, there was no significant drug treatment effect on the enlargement of lateral ventricle size.

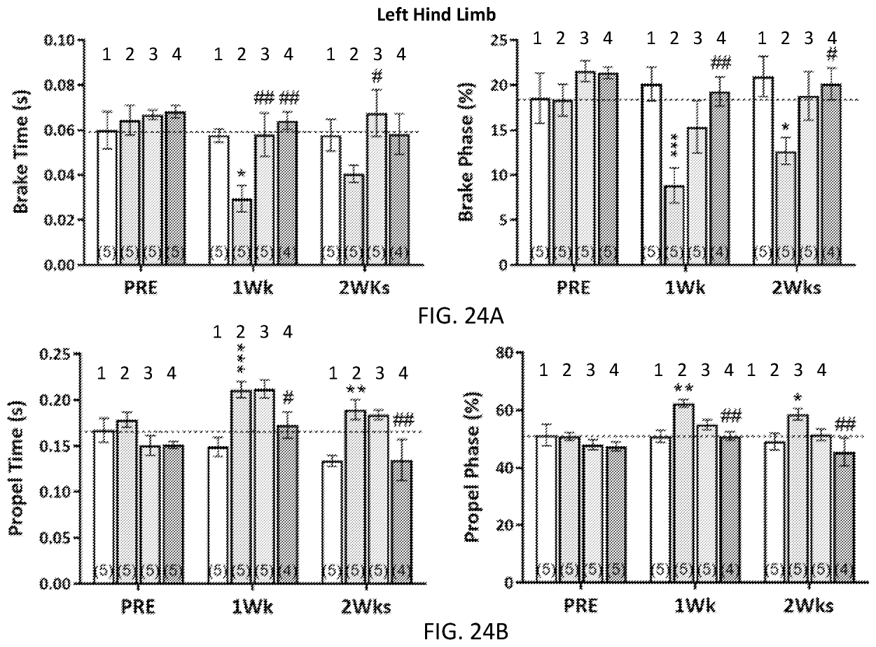

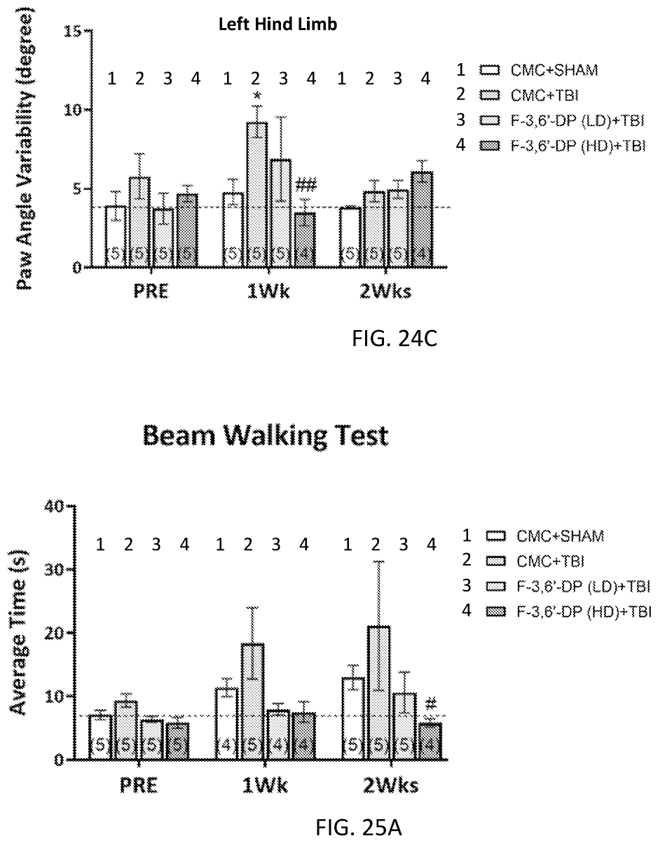

Compound 30 attenuated gait impairments caused by TBI. To determine whether compound 30-mediated mitigation in proinflammatory activity is behaviorally relevant, behavioral tests were measured before and after cerebral injury in mice with compound 30 or vehicle post treatment (FIG. 21). DigiGait analysis was performed to measure gait function in TBI and sham mice. As demonstrated in FIGS. 24A-24C, there were no significant differences before injury (PRE), and TBI significantly decreased the time duration of the initial paw contacted to maximum paw contact (brake time) and the percent of the total time duration that the paw was in brake phase at 1 and 2 weeks after injury (*p<0.05, ***p<0.001, comparing with CMC+sham group) (FIG. 24A). TBI mice treated with compound 30 (low dose, LD, 14.78 mg/kg) significantly mitigated the deficit of brake time cause by TBI at 1 and 2 weeks after TBI (#p<0.05, ##p<0.01, comparing with CMC+TBI group), and the impairment of brake phase was ameliorated in compound 30 high dose (HD) treatment group (29.57 mg/kg) (#p<0.05, ##p<0.01, comparing with CMC+TBI group). The time duration of the maximum paw contacted to just before the paw left the belt (propel time) and the percent of total duration that paw was in the propulsion phase were significantly elevated by TBI (**p<0.01, ***p<0.001, comparing with CMC+sham group) (FIG. 24B), and treatment of compound 30 (high dose (HD), 29.57 mg/kg) significantly mitigate propel time and % of propel phase elevated by TBI (#p<0.05, ##p<0.01, comparing with CMC+TBI group). The standard deviation of the paw angle was significantly increased at 1 week after TBI (*p<0.05, comparing with CMC+sham group) and F-3,6′-DP (high dose (HD), 29.57 mg/kg) can significantly alleviate this impairment induced by TBI (##p<0.01, comparing with CMC+TBI group) (FIG. 24C).

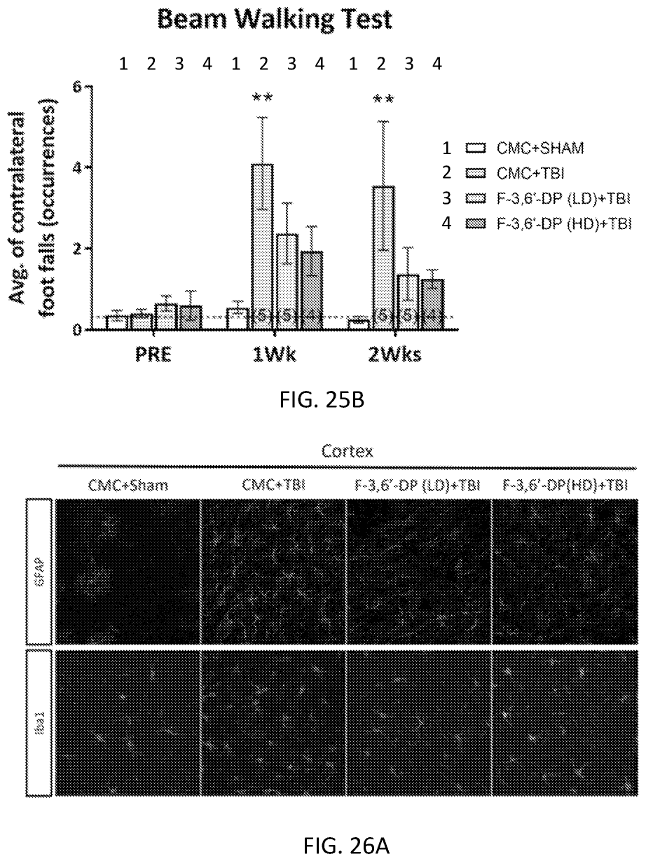

The beam walking test was used to measure motor coordination in TBI and sham mice and included two parameters (average transit time (FIG. 25A) and number of contralateral foot falls (FIG. 25B) during the traversing the beam). The average time of the test largely increased after TBI but it did not reach significant difference (FIG. 25A). The number of contralateral foot falls significantly increased after TBI (**p<0.01, comparing with CMC+sham group). Although clear reducing trends of both parameters in compound 30 treated groups were observed, only the average time of compound 30 high dose (HD, 29.57 mg/kg) treatment group reached significant different (#p<0.05, comparing with CMC+TBI group) (FIG. 25A).

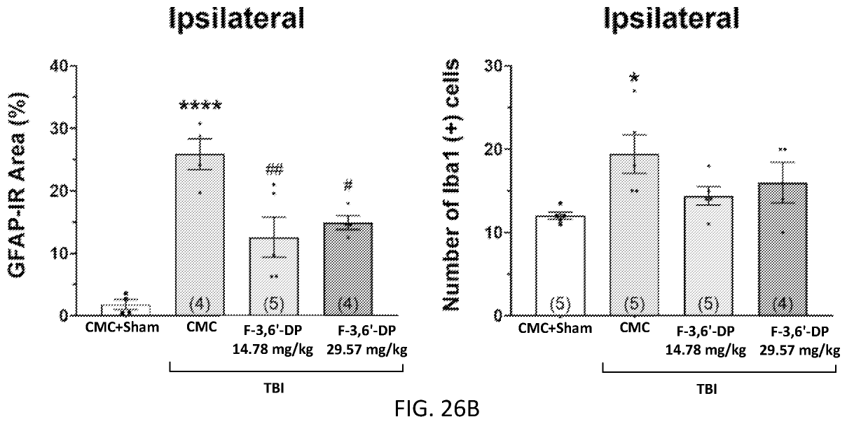

Compound 30 treatment ameliorated the activation of microglia and microglia induced by TBI. To determine the activity of neuroinflammation in brain after TBI, the number of astrocytes were measured by GFAP immunostaining in cortex, and microglia were stained by Iba1. After TBI, significant induction of the number of astrocytes and microglia was observed (*p<0.05, ****p<0.0001, comparing with CMC+sham group) (FIGS. 26A, 26B). Treatment with compound 30 (14.78, 29.57 mg/kg) significantly decreased the number of astrocytes elevated by TBI (#p<0.05, ##p<0.01, comparing with CMC+TBI group) (FIGS. 26A, 26B).

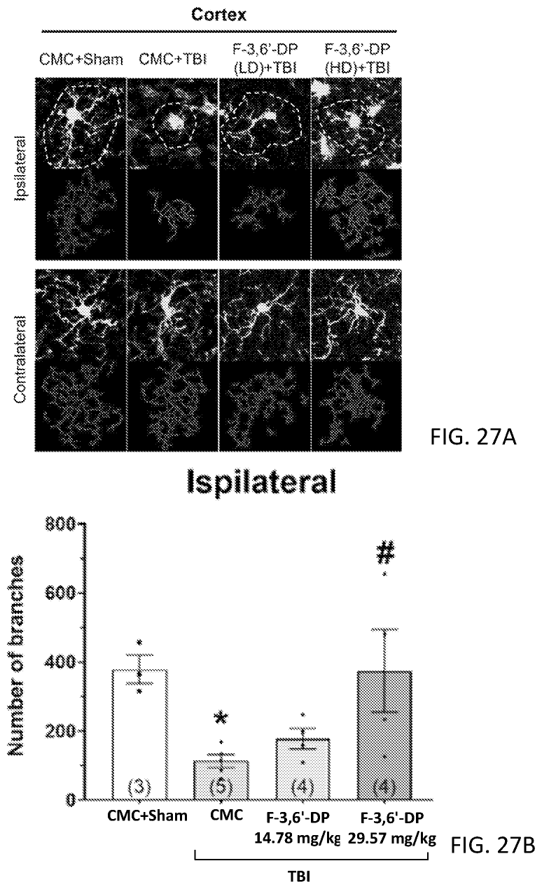

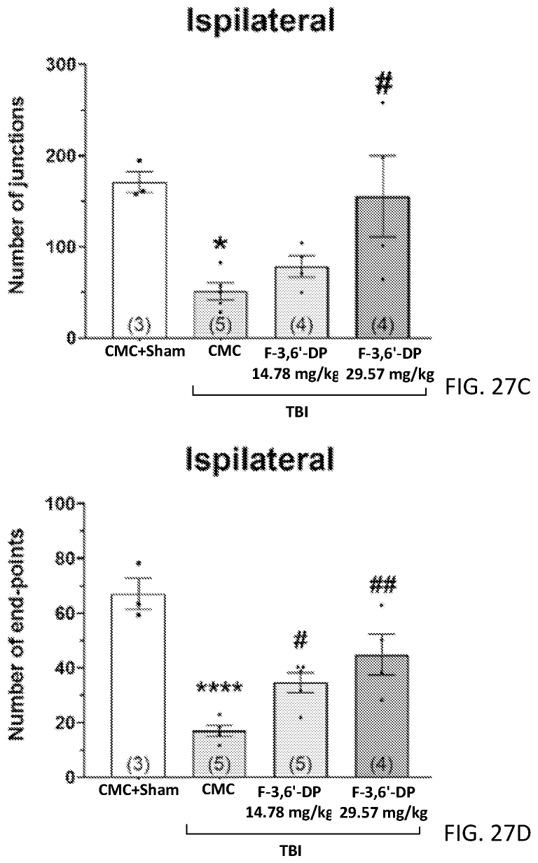

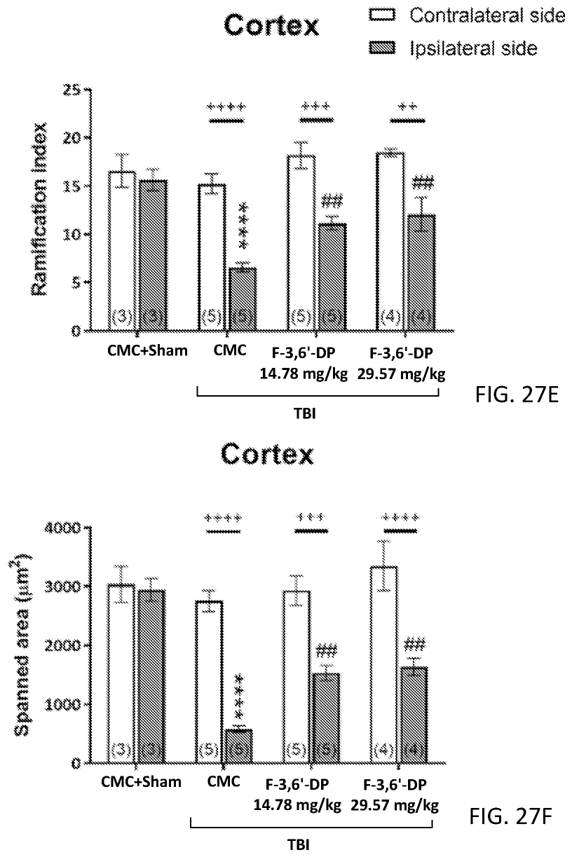

The morphology of microglia indicates different states of activation. The ramified form, with smaller somata and wider processes extensions, is considered as “resting” condition of microglia. At this state of microglia, they conduct surveying role around their microenvironment and helping with neuronal cell's function. After neuronal injury, microglia will transform into amoeboid morphology by retracting their processes and extending the protrusions to become “active” form (Davis et al., Sci Rep 2017, 7:1576). The microglia morphology was assessed by quantifying the images of Iba1 positive cells using MotiQ plugin under FIJI software. TBI dramatically changed the morphology of microglia from ramified from to amoeboid form in the ipsilateral cortex region of brain and without affecting the microglia in contralateral side (FIG. 27A). The number of branches (FIG. 27B), junctions (FIG. 27C), endpoints (FIG. 27D) of microglial processes were significantly decreased by TBI (*p<0.05, ****p<0.0001, comparing with CMC+sham group) and compound 30 (14.78, 29.57 mg/kg) could significantly rescue the morphological changes of microglia caused by TBI (#p<0.05, ##p<0.01, comparing with CMC+TBI group) (FIGS. 27B-27D). Ramification index, the ratio of surface area to volume, and spanned area were used to indicate the morphology of microglia. Unilateral impact on cortex will reduce the ramification index and spanned area of microglia specifically in ipsilateral side without disturbing contralateral side (****p<0.0001, comparing with CMC+sham group) (FIGS. 27E, 27F). Treatment with compound 30 significantly ameliorated the morphological alteration of microglia induce by TBI (##p<0.01, comparing with CMC+TBI group) (FIGS. 27E, 27F).

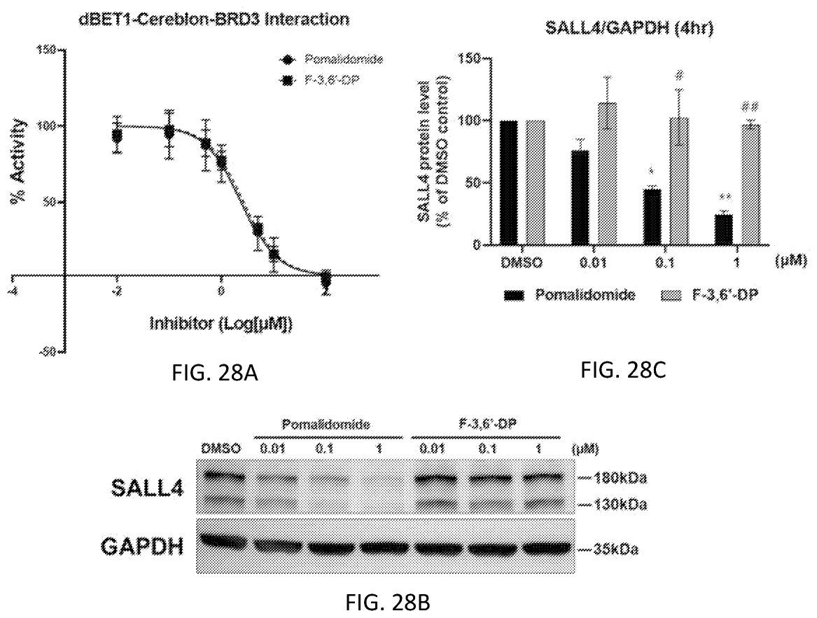

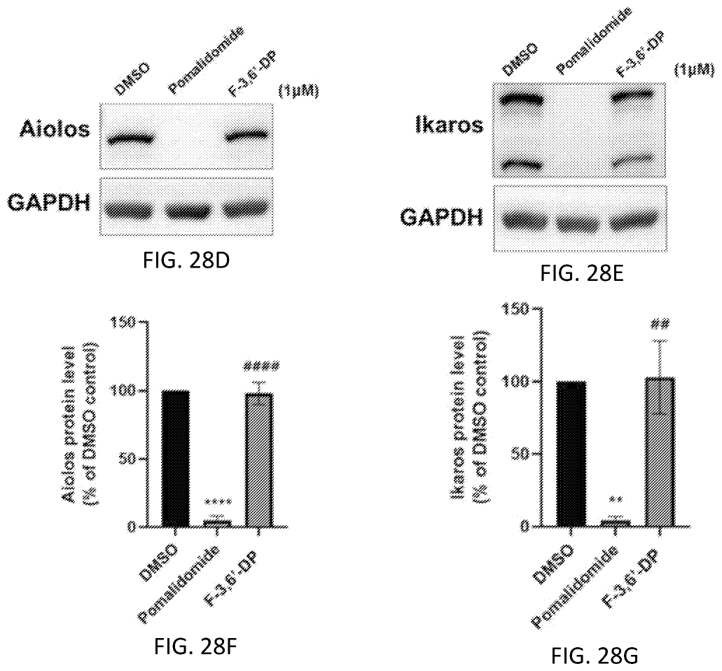

Compound 30 does not bind cereblon and neo-substrates. The binding of pomalidomide and compound 30 to cereblon was examined using a cereblon/BRD3 binding FRET assay (FIG. 28A). A concentration-dependent evaluation of binding between pomalidomide and cereblon provided an IC50 value of 2.38 μM whereas the IC50 of F-3,6′-DP was 2.66 μM, indicating that both agents potently bind to cereblon (with the concentration-dependent curves largely superimposing one another). The thalidomide analog-mediated degradation of SALL4 in human Tera-1 cells (0.01, 0.1, 1 μM of pomalidomide and compound 30) and of Aiolos and Ikaros in the human multiple myeloma MM1.S cell line (1 μM of pomalidomide and of compound 30) was evaluated by Western blotting with quantification relative to GAPDH expression (FIGS. 28B-28G). Whereas pomalidomide dramatically lowerered SALL4, Aiolos, and Ikaros levels, compound 30 had no significant effect on these cereblon neo-substrates.

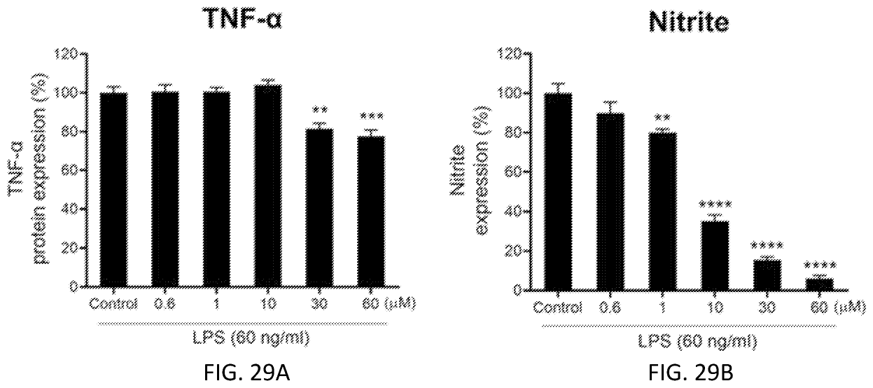

Compound 30 mitigates LPS-induced inflammation in RAW 264.7 cells. Cultured RAW 264.7 cells were pretreated with either vehicle or F-3,6′-DP (0.6-60 μM) and challenged with LPS (60 ng/mL) 1 h later. At 24 h following LPS exposure, cellular viability, nitrite (a stable marker of NO generation), and TNF-α levels were quantified. F-3,6′-DP was well tolerated and maintained cellular viability at >90% across all concentrations evaluated, and significantly lowered LPS-induced elevations in nitrite and TNF-α levels (FIGS. 29A, 29B).

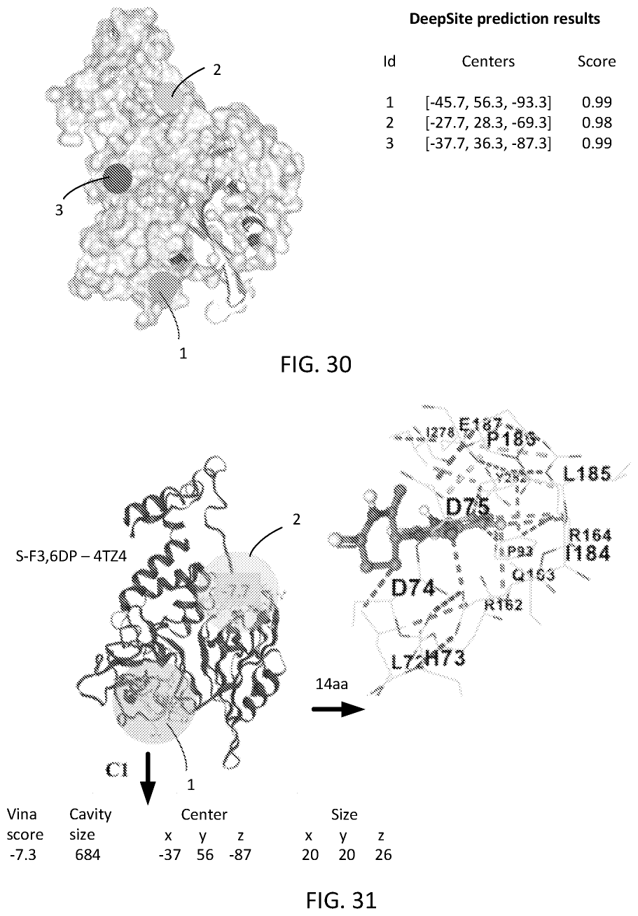

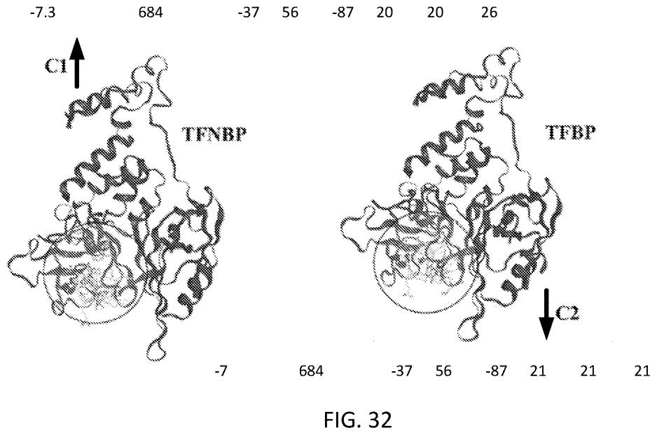

Binding of compound 30 to docking pocket of human cereblon. To gain insight into the potential interactions and binding of F-3,6′-DP with cereblon and how this might differ from the conventional IMiDs thalidomide and pomalidomide, molecular modeling studies were performed utilizing the X-ray crystallographic structures of human cereblon (obtained from prior studies involving the IMiD lenalidomide (https//www.rcsb.org/structure/4TZ4, accessed on 27 Jul. 2022) and those of the three compounds evaluated here. These molecular modeling studies open the possibility of human cereblon having more than a single binding domain for IMiDs by predicting that up to 3 potential pharmacophore sites may serve as pockets for drug binding. These potential pockets are numbered according to their scores (FIG. 30). The docking prediction suggests that thalidomide and pomalidomide binding/interaction was predicted for pocket number 1, which aligns with the pocket determined by x-ray crystallography studies (Chamerlain et al., Drug Discov Today Technol. 2019, 31:29-34; Boichenko et al., ACS Omega 2018, 3:11163-71; Asatsuma-Okumura et al., Pharmacol Ther. 2019, 202:132-9; Matyskiela et al., Nat Struct Mol Biol. 2020, 27:319-22). Vina scores were calculated as −9.1 and −9.5, respectively, for the S stereoisomer form of thalidomide and pomalidomide, with the difference in scores arising from pomalidomide's predicted interaction with more amino acids than thalidomide, associated with a better Vina score for pomalidomide. In contrast, compound 30 was predicted to engage within pocket number 2, with a Vina score of −7.7 (FIG. 31). Notably, compound 30 was predicted to also bind in pocket 1, with a lower ranking and prediction Vina score of −7.3 (FIG. 31). These model-associated differences in docking pockets and the nature of bonds that interact with amino acids within the pharmacophore may influence and potentially underpin the differences reflected in the cereblon binding actions on the neo-substrates SALL4, Ikaros, and Aiolos evident in FIGS. 28B-28G.

### Discussion and Conclusion

Accumulating evidence suggests that neuroinflammation is considered the critical factor among neurodegeneration disorders (Webster et al., J Neuroinflammation 2017, 14:10; Frankola et al., CAN Neurol Disord Drug Targets 2011, 10:391-403; Wilcock et al., J Neuroinflammation 2013, 10:84; Clark et al., J Neuroinflammation 2016; 13:326; Degan et al., Curr Pharm Des. 2018, 24:1485-1501; Ranshoff, Science 2016, 353:777-783). Within hours after TBI, a variety of cytokines will be produced and released to induce local immune cells activation and attracted more peripheral immune cells to the local area, including neutrophil, monocyte infiltration, microglial, astrocytic activation, and reactive oxygen species (ROS) production (Webster et al., J Neuroinflammation 2017, 14:10). Epidemiological data indicates great potential protected effect of treatment with non-steroidal anti-inflammatory drugs (NSAIDs) against the development of neurodegeneration disorders, gene polymorphisms of several pro-inflammatory cytokines seem to amend the risk of getting AD and PD (Klegeris et al., Curr Alzheimer Res 2005, 2:355-365; Vlad et al., Neurology 2008; 70:1672-1677; Zhang et al., Front Aging Neurosci 2018, 10:83), but there have largely failed in randomized clinical trials of anti-inflammatory compounds in neurodegeneration diseases (Gyengesi et al., Nat Rev Neurol 2020, 16:131-132; Stein et al., Neuropharmacology 2019, 147:66-73). Among many accountable aspects that could elucidate this failure is that the cause of TBI involving multiple molecular pathology mechanisms, and their temporal profiles and outcomes are different between preclinical animal models and human TBI. Therefore, instead of targeting specific mechanism as target-based drug screening strategy, phenotypic screening might be a good approach to determine potential compounds Swinney, et al., Nat Rev Drug Discov 2011, 10:507-519; Swinney, Clin Pharmacol Ther 2013; 93:299-301) that can ameliorate crucial parameters related to TBI, such as behavioral impairments, neuronal death, microglial phenotypic alteration. In consideration of microglial activation appearing in many neurodegeneration disorders, like TBI, and accompanying with a variety of pro-inflammatory cytokines or chemokines expression (Edwards, et al., Front Neurol 2020, 11:348; Sun et al., Front Neurol 2019, 10:1120), such as TNF-α, IL-1β, IL-6, cytokine-suppressive anti-inflammatory drugs (CSAIDs) seem to have great potential for treatment approach. In this study, CSAID, compound 30, demonstrated potent activity in reducing pro-inflammatory cytokines and chemokines, such as TNF-α, KC/GRO (CXCL1), IL-6, and free radicals, like nitric oxide, ameliorating microglial and astrocytic activation after TBI.

The physiological and pathophysiological functions of microglia, the level of TNF-α expressed by microglia is very crucial in regulating the homeostatic maintenance of neuronal activities (Olmos et al., Mediators Inflamm 2014. 2014:861231; Clark et al., Semin Immunopathol 2017, 39:505-516). The activation of microglia after TBI usually occurs within 24 hours (Webster et al., J Neuroinflammation 2017, 14:10; Wang et al., J Neuroinflammation 2016, 13:168; Knoblach, et al., J Neuroimmunol 1999, 95:115-125) and will extend from weeks to months (Scott et al., Brain 2018, 141:459-471). In response to various microenvironments, microglia are morphologically and functionally dynamic cells that can alter forms from ramified to completely lacking its processes with a larger cell body (Amoeboid), usually associated with phagocytic functions (Morrison et al., Sci Rep 2017, 7:13211; Donat et al., Front Aging Neurosci 2017, 9:208; Choi et al., Sei Rep 2022, 12:1806). Early microglia activation after TBI may conduct the restoration process of homeostasis in brain. However, if the activity of microglia remains chronically activated, presenting activated morphology and producing pro-inflammatory mediators, will result in extended brain tissue impairment and giving potential to neurodegeneration (Donat et al., Front Aging Neurosci 2017, 9:208). The balance of activation status of microglia is relatively dynamic at various timepoints after TBI, but their distribution, morphological changes and functional phenotypes can provide us information to clarify the status of neuroinflammation in preclinical animal models and in humans. The strength of animal models is that it can allow us to manipulate their genetic and pharmacological profile, in order to determine their character. In this study, we found that the morphological changes of microglia after TBI, not only occurs in cortex of local injury site, but also in thalamus (data not shown). This finding echoes the discovery in clinical, which has found increasing activity of microglia in thalamus, putamen and midbrain after TBI (Donat et al., Front Aging Neurosci 2017, 9:208; Kobayashi et al., J Cereb Blood Flow Metab 2018, 38:393-403; Ramlackhansingh et al., Ann Neurol 2011, 70:374-383; Kumar et al., J Neuroinflammation 2012, 9:232). The result may be explained by the thalamocortical radiations, the nerve fibers between thalamus and cerebral cortex, contributing the sensory or motor functions from thalamus to distanced areas of cortex through relay neurons (George, “Neuroanatomy, thalamocortical radiations,” Statpearis 2022. Treasure Island, FL). It is implied that even subtle neuronal damage, as may occur in normal aging, may trigger the morphological changes or accumulation of secondary microglial activation in remoted areas from injury happening site. Furthermore, the number of microglia can remarkably be elevated by TBI specifically in ipsilateral side, this phenomenon appears not only in cortex (FIG. 25B) but also in thalamus (data not shown). After compound 30 treatment, subtle reduction, but not significant, of the number of microglia induced by TBI was detected. However, the microglial morphological analysis showed dramatic differences between vehicle and compound treatment groups in TBI mice (FIGS. 26A-26F). This indicates that information of the amount and morphology of microglia provides us different concepts of microglia activity during neuroinflammation, both contributing the balance of microglial activation status.

Gait impairment is a classical marker in clinical TBI population (Williams et al., Arch Phys Med Rehabil 2009, 90:587-593; Dever et al., Sensors (Basel) 2022, 22; Williams et al., J Head Trauma Rehabil 2015, 30: E13-23). The gait analysis in animal models of TBI is important, owing to most injury models directly affect motor circuits that control gait function, and it is clinical related symptom that has potential to translate from bench to bedside. Studies have shown different severity of CCI in rodent TBI model can induce both cognitive and motor impairments differently Osier et al., Brain neurotrauma: Molecular, neuropsychological, and rehabilitation aspects 2015, Boca Raton, FL; Yu et al., Brain Res 2009, 1287:157-163). The DigiGait system can provide detailed gait parameters by using a camera detecting system under treadmill and identifying several steps of gait across animals and trials. In this study, the majority gait deficits induced by TBI occurred in contralateral (left) side, especially at left hindlimb, which echoed the data from previous studies (Reed et al., Behav Brain Res 2021, 405:113210; Sashindranath et al., Behav Brain Res 2015, 286:33-38) and the observation of the footfalls during beam walking test. It is notable that craniotomy itself can induce certain parameters change of gait based on previous study (Sashindranath et al., Behav Brain Res 2015, 286:33-38), so the sham group only received skin incision without craniotomy. In fact, it is also broadly debated whether receiving craniotomy is an ideal control for CCI model of TBI (Cole et al., J Neurotrauma 2011, 28:359-369). In the inventors' experience, subtle elevation of astrocytes and microglia at the surface area was still observed of cortex around craniotomy site (data not shown), which is related to previous study (Lagraoui et al., Front Neurol 2012; 3:155). The mechanism of the gait impairment cause by TBI is still not clear. However, neuronal networks of spinal cord directly involve the movements of limbs and coordination of gait during locomotion (Frigon, J Neurophysiol 2017, 117:2224-2241) and cortical injuries after CCI can induce microglia activation through corticospinal tract (Jacobowitz et al., Brain Res 2012, 1465:80-89), which provides a potential pathway to dig in for understanding the mechanism behind gait impairments after TBI.

The question arises as to why conventional IMiDs, such as thalidomide and pomalidomide, bind to cereblon and trigger the degradation of the neo-substrates SALL4, Ikaros, and Aiolos, whereas structurally close analogs, such as compound 30 and 3,6′-DP of former studies (Lecca et al., Alzheimers Demin. 2022, 18 (11): 2327-40; Tsai et al., Pharmaceutics 2022, 14:950) bind cereblon but do not then trigger alike neo-substrate degradation. Molecular modeling studies were thus undertaken to define conceivable factors underpinning the differential cereblon-related actions of compound 30 versus the clinically approved IMiDs thalidomide and pomalidomide, and to provide new potential directions for future evaluation. These modeling studies suggest that further docking pockets may exist for IMID-like drugs on the surface of cereblon, in addition to the pocket reported in X-ray crystallography studies of cereblon co-incubated with lenalidomide, thalidomide, and pomalidomide (Chamberlain et al., Drug Discov. Today Technol. 2019, 31:29-34; Matyskiela et al., Nat. Struct. Mol. Biol. 2020, 27:319-22), which aligns with pocket number 1 in our studies (FIG. 30). The modeling studies suggest the prospect that the S-stereoisomer of compound 30 differentially interacts with key amino acids within a separate adjacent pocket (FIG. 31, pocket number 2), which provided compound 30 its best ranked binding scores as compared to pocket number 1 (FIG. 30), where it was also predicted to bind with a lower ranking. Such differential binding may support the results of the cereblon/BRD3 binding FRET assay through possible allosteric interactions of binding pocket number 2 with binding pocket number 1, but not support the interaction of SALL4, Ikaros, and Aiolos with compound 30 bound within pocket number 2. Binding and ensuing neo-substrate degradation are known to occur with clinically approved conventional IMiDs bound in pocket number 1 and, moreover, provide an avenue for future research. Likewise, it would be interesting to evaluate whether similar differences exist in the interaction of the IMiDs with known inflammatory mediators involved in TBI and are known to chronically persist in neurodegenerative disorders in order to unravel the potential mechanism of actions of conventional, as well as new generation IMiDs.

In summary, neuroinflammation is a potential target of drug development for TBI and other neurodegenerative disorders. Based on the chemical structure of thalidomide, the drug class of immunomodulatory drugs (IMiDs), and its clinical available structural analogs, pomalidomide, lenalidomide, conduct efficient activity in reducing TNF-α expression during inflammation and show novel treatment strategy for drug repurposing application, but unfortunately have several adverse effects (Vargesson, Birth Defects Res c Embryo Today 2015, 105:140-156; Vargesson, J Hand Surg Eur Vol 2019, 44:88-95). Compound 30, as a novel class of IMiDs, demonstrates great activities in ameliorating neuroinflammation and behavioral impairments induced by TBI in the mouse model without binding to cereblon and affecting the key proteins involved in antiproliferative, anti-angiogenic and teratogenic actions of the IMiD drug class (Chamberlain et al., Drug Discovery Today Technol 2019, 31:29-34; Ito et al., Int J Hematol 2016, 104:293-299; Stewart, Science 2014, 243:256-257). Therefore, compound 30, as a novel candidate compound, deserves further investigation in drug development of neurodegeneration diseases or disorders involving inflammation.

Compound 30 can reduce the expression of free radicals, like nitric oxide, in vitro, and pro-inflammatory cytokine, IL-6 and chemokine, KC/GRO (CXCL1), in vivo. Notably, TNF-α, the key pro-inflammatory cytokine during inflammation, can be efficiently downregulated by compound 30. Compound 30 can reduce the gait impairment and lesion area induced by TBI and ameliorate the number of astrocytes, morphological change of microglia after TBI, which are the hallmark of neuroinflammation. The number of microglia induced by TBI did not significantly reduce with compound 30, suggested that the quantity and morphology of microglia play different role in neuroinflammation.

### Example 7

### Teratogenicity and Cereblon Interactions of TFBP and TFNBP

### Methods

Chick embryology and analysis: Chicken eggs were obtained from Henry Stewart & Co Ltd, Norfolk UK. All work with chicken embryos obeyed UK Home Office regulations and followed guidelines, standards and practices governed by the University of Aberdeen Ethics Committee (Scotland, UK). Each working solution of TFBP contained DMSO at 0.5% (3.5 μM TFBP), 1% (7.0 μM TFBP) and 2% (14.0 μM). Embryos were incubated at 37° C. for the required time period to reach E2.5 and E4 (early and mid-developmental stages, respectively). Eggs were then opened and the embryonic membranes protecting the embryos were removed with forceps. Chicken embryos typically lie on one side, so the left side is directly against the yolk and the right side can be observed. Drug (TFBP) or Control (DMSO alone) solutions were applied in 100 μL aliquots over the middle of the embryo on its right side. Embryos were left at room temperature for 20 min before being replaced in a 37° C. incubator. Due to the limited diffusion of drugs when applied to the right side of an embryo, the right side is considered the ‘treatment’ side and the left (facing internally towards the yolk sack) is considered normal after treatment and can, thereby, act as an internal control.

Docking pockets and predictions of TFBP and thalidomide structural analog interactions with cereblon: Assessments of prospective docking pockets and docking predictions for S enantiomeric forms of TFBP (compound 14), TFNBP (compound 16), and thalidomide-like drugs on the structure of cereblon were investigated using automated software (Martínez-Rosell et al., J Chem Inf Model. 2017, 57:1511-6). This was followed by a cavity-based blind drug docking prediction utility (Jiménez et al., Bioinformatics 2017, 33:3036-42) to appraise the characteristics of the computed drug docking predictions of these IMiDs. The drug docking pockets and the binding differences between compound 14, compound 16, thalidomide and pomalidomide in cereblon were determined for the best scoring attributes of these chemical agents. In short, the crystal structure of human cereblon in complex with DDB1 and lenalidomide (4TZ4: https://www.rcsb.org/structure/4TZ4) was downloaded in PDB format from the PDB database. The chain C (human cereblon) was separated from the remainder of the crystal structure complex and was utilized in docking predictions for the S enantiomeric forms of the study compounds. The S enantiomer was chosen as former x-ray crystallographic studies have reported that this enantiomer of thalidomide-like IMiDs better binds cereblon (Biochenko et al., ACS Omega 2018, 3:11163-71), notwithstanding that molecular modelling computational data does not necessarily simulate or fall in line with all experimental data from prior x-ray crystallographic studies (Adelusi et al., Informatics Med. 2022, 29:100880). Playmolecule, an automated server that employs a software DeepSite (Zárate et al., Front Aging Neurosci. 2017, 9:430; Martínez-Rosell et al., J Chem Inf Model. 2017, 57:1511-6) to establish the core binding sites, was used to simulate potential interactions between compound 14, compound 16, or thalidomide-like compounds with human cereblon. An automated docking software (Liu et al., Acta Pharmacol Sin. 2020, 41:138-44) was used to investigate potential similarities and differences in the pharmacophore pocket engaged by the test drugs. Briefly, two files were uploaded that included the C-chain (human cereblon) of the PDB ID 4TZ4 without lenalidomide and damaged DNA binding protein 1 (DDB1) and the drugs individually in their PDB formats to the Docking server (Ibid.). For docking pocket predictions, the results appear as the number of preferential pockets determined with their relevant scores. Results of docking were collected with individual Vina scores, cavity sizes, docking centers, poses and sizes of predicted cavities for the drugs noted above. The resulting drug-cereblon complexes were visualized using the drug discovery studio visualizer software BIOVIA (Martínez-Rosell et al., J Chem Inf Model. 2017, 57:1511-6).

Statistical analysis: Data were evaluated between groups with one-way analysis of variance (ANOVA) followed by Dunnett's posthoc tests (GraphPad Prism® 7, San Diego, CA, USA) when appropriate for multiple comparisons. The behavioral data were evaluated between groups with two-way ANOVA followed by Dunnett's posthoc test. Grubb's test was used to identify and remove outliers. Bar graphs are presented as mean±SEM values. A p value of <0.05 was considered statistically significant, and levels of significance are provided in the legend for each individual figure.

Results and Discussion: To evaluate prospective interactions and the potential binding of TFBP (compound 14), TFNBP (compound 16) and structural analogues of thalidomide with chain C of human cereblon and how this could contrast with the conventional IMiDs, molecular modeling studies were implemented utilizing the x-ray crystallographic structures of cereblon obtained from prior studies involving the IMiD lenalidomide (https://www.rcsb.org/structure/4TZ4). Pocket determination within cereblon forecast three top ranked pharmacophores with their attributes (FIG. 30). The docking prediction suggests that best thalidomide and pomalidomide binding/interaction is for pocket number 1, which aligns with the pocket determined by x-ray crystallography studies (Chamerlain et al., Drug Discov Today Technol. 2019, 31:29-34; Boichenko et al., ACS Omega 2018, 3:11163-71; Asatsuma-Okumura et al., Pharmacol Ther. 2019, 202:132-9; Matyskiela et al., Nat Struct Mol Biol. 2020, 27:319-22). Vina scores were calculated as −9.1 and −9.5, respectively, for the S stereoisomer form of thalidomide and pomalidomide, with the difference in scores arising from pomalidomide's predicted interaction with more amino acids than thalidomide, associated with a better Vina score for pomalidomide. By contrast, compounds 16 and 14 were projected to occupy the same classic pharmacophore (pocket #1) but with Vina scores of −7.3 and −7.0, respectively (FIG. 29). Notably, compounds 16 and 14 were predicted to also bind in proposed pockets #2 and #3 (FIG. 28). The binding pocket preferences and Vina scores of compounds 16 and 14 reflect a poor interaction probability of these compounds within the classic thalidomide binding pharmacophore (pocket #1). These differences in docking pocket binding interactions of the evaluated IMiDs and, in particular, with the amino acids within the pharmacophore, not only determine the strength of binding interactions with cereblon but also the orientation of the IMID within the pocket and its potential binding to neo-substrates such as SALL4.

As an initial in vivo evaluation of teratogenicity, compound 14 was applied to the right side of chicken embryos at an early (E2.5) and mid (E4) developmental stage, and embryos were examined at 24, 48 and 72 h, morphologically. As detailed in Table 12, three compound 14 doses (3.5, 7.0 and 14.0 μM) were evaluated, together with DMSO alone. Whereas the lower compound 14 dose proved to be well tolerated, the two higher doses resulted in an increasing death rate. No effects on embryo development were evident at the compound 14 3.5 and 7.0 μM doses or after DMSO alone; the deaths observed (compound 14 7.0 μM) appeared to be due to damage of the embryonic membranes that were removed before application of the drugs—as the embryos, themselves, appeared normal. A single embryo challenged with compound 14 14.0 μM had micro-opthalmia of the eye (Table 12). Notably, this was found in the eye facing away from the site of drug application (i.e., on the control side). No other developmental anomalies were evident, and the embryo was alive at the end of the experiment. In this light, the micro-opthalmia could potentially be a spontaneous malformation, as the three deaths seen at the 14.0 μM dose occurred quite late in development (around 48-72 h after drug application) and those embryos appeared developmentally and morphologically normal. All DMSO control embryos (N=2) were normal.

TABLE 12

| | Age (days) | | | | | | | |
|---|---|---|---|---|---|---|---|---|
| TFBP Dose | E2.5 | | | | E4 | | | |
| (μM) | N | Normal | Abnormal | Dead | N | Normal | Abnormal | Dead |
| 3.5 | — | — | — | — | 7 | 7 | 0 | 0 |
| 7.0 | 6 | 4 | 0 | 2 | 7 | 4 | 0 | 3 |
| 14.0 | 6 | 2 | * | 3 | — | — | — | — |

Classical thalidomide-like drugs can hence potentially induce anti-inflammatory actions mediated through cereblon E3 ubiquitin ligase-dependent and -independent mechanisms. Unlike thalidomide and pomalidomide, compounds 14 and 16 do not bind within the classical thalidomide binding domain (i.e., pocket #1) of human cereblon, as evaluated by cereblon/BRD3 binding FRET assay (FIG. 20A, Example 5) and molecular modeling (FIG. 31). Consequent to this, and unlike classical IMiDs, compounds 14 and 16 interactions with human cereblon do not support SALL4 degradation, and thus anti-inflammatory action of these novel IMiDs is mediated via a cereblon independent mechanism in the absence of overt teratogenicity, as evaluated in compound 14 challenged chicken embryo preliminary studies (Table 12) that previously have shown sensitivity to thalidomide (Therapontos et al., PNAS U.S. Pat. No. 2,009,106:8573-8). In further support of this, key amino acid sequence differences exist between rodent and human cerebelon, with a particularly critical one occurring within the thalidomide binding domain (pocket #1). Whereas mouse cereblon is 95% homologous to the human form and can bind to thalidomide, degradation of SALL4 and related neo-substrates does not occur in the rodent and accounts for the lack of teratogenicity/antitumor action of classical IMiDs in rodents vs. their activity in humans (and chicken embryos), which can be conveyed to rodents by site-directed mutagenesis in the generation of cereblon-humanized mice (Fink et al., Blood 2018, 132:1535-44; Gemechu et al., PNAS U.S.A. 2018, 115:11802-7). In contrast, thalidomide and conventional IMiDs induce anti-inflammatory actions in both wild-type and cereblon-humanized mice (Fink et al., Blood 2018, 132:1535-44), likewise indicating presence of a cereblon-independent anti-inflammatory pathway.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A compound having a structure according to Formula I, or a stereoisomer or pharmaceutically acceptable salt, solvate, or hydrate thereof: 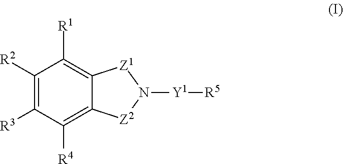
where
R¹ is —X, —N(R′)(R″), —NO₂, or —OH, wherein X is halo, and R′ and R″ independently are H or C₁-C₃ alkyl;
R²-R⁴ independently are —X or —H;
R⁵ is 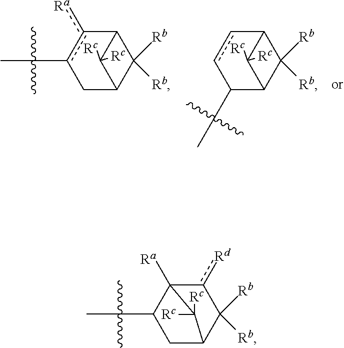
where each bond represented by “ [symbol] ” is a single or double bond as needed to satisfy valence requirements;
R^{a} is —CH₃, —H, —CH₂OH, or —CH₂;
each R^{b} independently is H or —CH₃;
each R^{c} independently is —CH₃ or —H;
R^{d} is —H, —OH, or ═O;
Y¹ is a bond, —CH₂—, or —CH(CH₃)—; and
Z¹-Z² independently are C(O) or C(S),
wherein at least one of R²-R⁴ is —X; or
the compound is select from 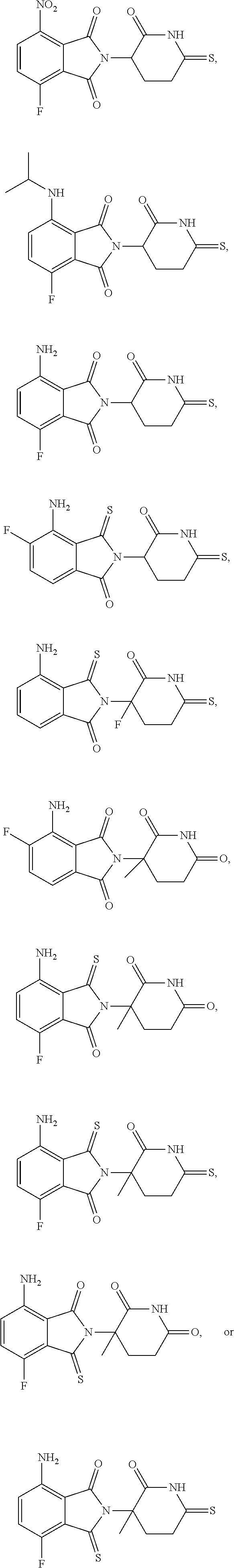
or a stereoisomer, pharmaceutically acceptable salt, solvate, or a hydrate thereof.

2. The compound of claim 1, wherein X is F.

3. (canceled)

4. The compound of claim 1, wherein:
R^{a} is —CH₃ or —H;
each R^{b} is —H or each R^{b} is —CH₃; and
each R^{c} is —CH₃ or each R^{c} is —H.

5. The compound of claim 4, wherein R⁵ is:

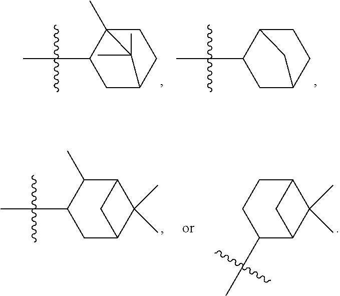

6. The compound of claim 5, wherein R⁵ is:

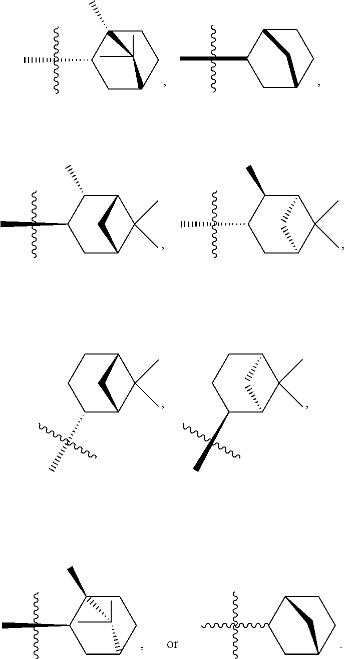

7. The compound of claim 1, wherein:
Z¹ and Z² are C(O) and R¹-R⁴ are —F; or
Z¹ and Z² are C(O), R¹ and R⁴ are —F, and R² and R³ are —H.

8-13. (canceled)

14. The compound of claim 1, wherein the compound is: 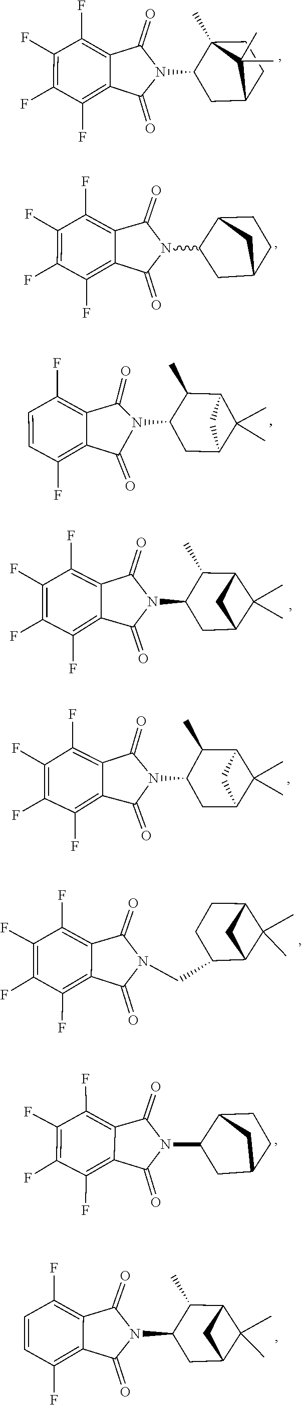 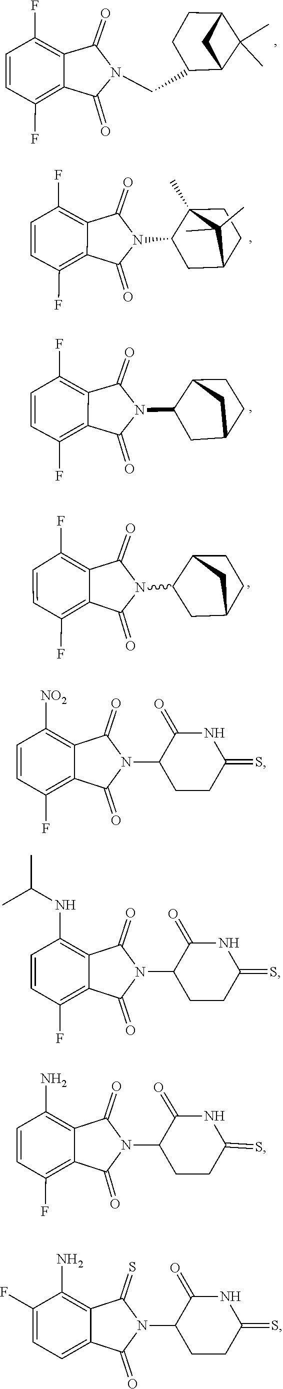 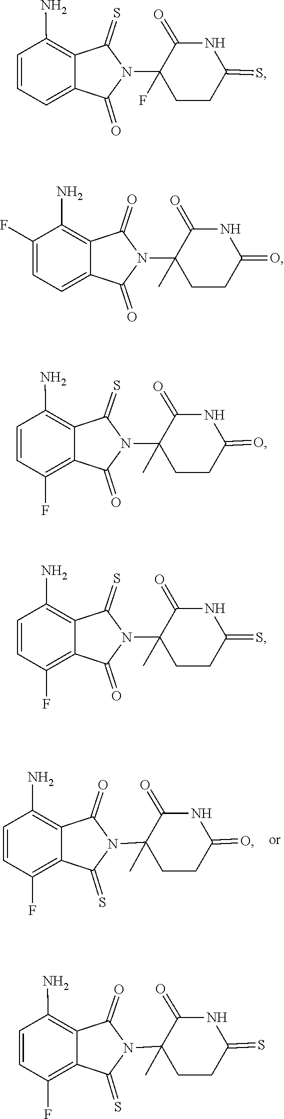
or a stereoisomer, pharmaceutically acceptable salt, solvate, or a hydrate thereof.

15. A pharmaceutical composition comprising:
a compound, or a stereoisomer, pharmaceutically acceptable salt, solvate, or a hydrate thereof, according to claim 1; and
a pharmaceutically acceptable carrier.

16. A method for inhibiting TNF-α activity, TNF-α synthesis, inflammation, inducible nitric oxide synthase (iNOS), SARS-CoV-2 virus, or any combination thereof, comprising contacting a cell with an effective amount of a compound or a stereoisomer, pharmaceutically acceptable salt, solvate, or hydrate thereof, according to claim 1.

17. The method according to claim 16, wherein contacting the cell with an effective amount of the compound or stereoisomer, pharmaceutically acceptable salt, solvate, or hydrate thereof, comprises administering to a subject a therapeutically effective amount of the compound or stereoisomer, pharmaceutically acceptable salt, solvate, or hydrate thereof, or a therapeutically effective amount of a pharmaceutical composition comprising the compound or stereoisomer, pharmaceutically acceptable salt, solvate, or hydrate thereof.

18. The method according to claim 17, wherein the subject has a traumatic brain injury (TBI), an inflammatory disorder, an autoimmune disorder, a neurodegenerative disease, a viral infection, or any combination thereof.

19. The method according to claim 18, wherein the subject has a TBI, neuroinflammation, a SARS-CoV-2 virus infection, Alzheimer's Disease, Parkinson's Disease, multiple sclerosis, amyotrophic lateral sclerosis, Huntington's Disease, a spinal cord injury, a stroke, human immunodeficiency virus dementia, cerebral amyloid angiopathy, tauopathy, peripheral neuropathy, macular degeneration, hearing loss, cochlear injury, epilepsy, a non-epileptic seizure disorder, depression, rheumatoid arthritis, immune arthritis, degenerative arthritis, celiac disease, glomerulonephritis, lupus nephritis, prostatitis, inflammatory bowel disease, pelvic inflammatory disease, graft-versus-host disease, interstitial cystitis, autoimmune thyroiditis, Graves' disease; autoimmune pancreatitis, Sjogren's syndrome, myocarditis, autoimmune hepatitis, primary biliary cirrhosis, autoimmune angioedema, bullous pemphigoid, discoid lupus erythematosus, erythema nodosum leprosum, sarcoidosis, pemphigus vulgaris psoriasis, POEMS syndrome, polymyositis, human immune deficiency virus/acquired immune deficiency syndrome, vasculitis, sarcopenia, or any combination thereof.

20. The method according to claim 18, where:
the subject has a TBI, neuroinflammation, or both; and
the compound is 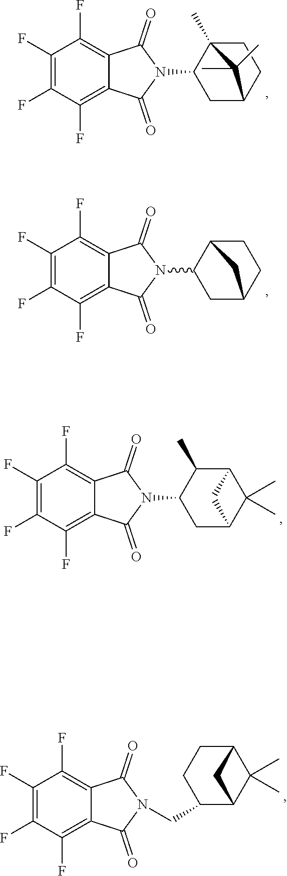
or a combination thereof.

21. The method according to claim 18, where:
the subject has a SARS-CoV-2 infection; and
the compound is

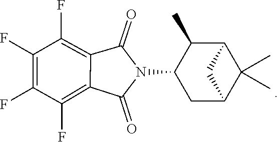 [SMILES: C[C@@H]1[C@@H](N2C(=O)c3c(F)c(F)c(F)c(F)c3C2=O)C[C@H]2C[C@@H]1C2(C)C]

22. The method of claim 16, comprising administering to a subject having aberrantly high TNF-α activity, aberrantly high nitrite activity, or both, a therapeutically effective amount of a compound or a stereoisomer, pharmaceutically acceptable salt, solvate, or hydrate thereof, or a pharmaceutical composition comprising the compound or stereoisomer, pharmaceutically acceptable salt, solvate, or hydrate thereof, wherein the compound is 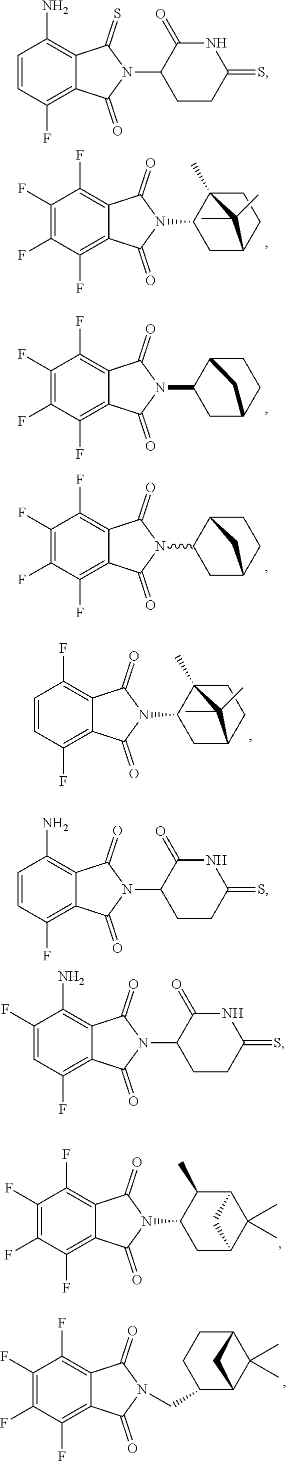
or any combination thereof.

23-32. (canceled)

33. The compound of claim 1, wherein R¹-R⁴ are —F.

34. The compound of claim 33, wherein Z¹ and Z² are C(O).

35. The compound of claim 1, wherein the compound is

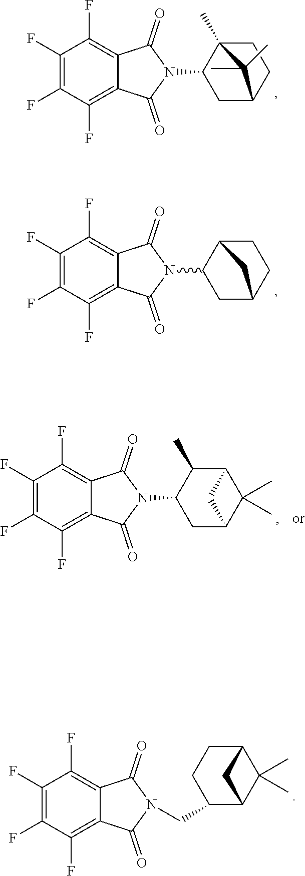

36. The method of claim 16, wherein the compound is [chemical structure]
